(19) United States
(12) Patent Application Publication
(10) Pub. No.: US 2024/0230483 A1
(43) Pub. Date: Jul. 11, 2024

(54) CHOP-FIX METHOD AND CHOPPING DEVICE FOR PREPARING BIOLOGICAL SAMPLES

(71) Applicant: 10x Genomics, Inc., Pleasanton, CA (US)

(72) Inventors: Jawad N. ABOUSOUD, Pleasanton, CA (US); Sharmila Chatterjee Bhattacharjee, San Jose, CA (US); Albert Kim, Oakland, CA (US); Ryan Stott, Oakland, CA (US); Joaquin Trosper-Torres, Sunol, CA (US); Fernandino Valdecanas, Pleasanton, CA (US)

(21) Appl. No.: 18/541,542

(22) Filed: Dec. 15, 2023

Related U.S. Application Data
(63) Continuation of application No. PCT/US2022/034661, filed on Jun. 23, 2022.
(60) Provisional application No. 63/213,908, filed on Jun. 23, 2021.
(60) Provisional application No. 63/214,043, filed on Jun. 23, 2021.
(60) Provisional application No. 63/349,064, filed on Jun. 4, 2022.

(51) Int. Cl. G01N 1/28; C12N 9/64; C12Q 1/6806; G01N 1/30

(52) U.S. Cl. CPC G01N 1/286; C12N 9/6491; C12Q 1/6806; G01N 1/30; G01N 2001/2873; G01N 2001/305

(57) ABSTRACT

The present disclosure provides methods for biological sample preparation and analysis that use a chopping and fixing treatment in the preparation of dissociated fixed cells from biological tissue for use in either bulk assays, or in single-cell/single-nucleus assays, such as partition-based assays. The disclosure also provides assay methods, including partition-based methods, for use of the fixed cells prepared from biological tissue that can optionally be used in combination with un-fixing treatments. Kits comprising dissociation reagents, un-fixing agents, and other assay reagents for use in the methods are also provided.

### CROSS REFERENCE TO RELATED APPLICATIONS

This application is a continuation of International Application No. PCT/US2022/034661, filed Jun. 23, 2022, which claims the benefit of priority to United States Provisional Application Nos. 63/213,908, filed Jun. 23, 2021, 63/214,043, filed Jun. 23, 2021, and 63/349,064, filed Jun. 4, 2022, each of which is incorporated herein by reference in their entirety.

### FIELD

The present disclosure relates generally to methods and devices for preparing biological samples of fixed cells from tissue for use in bulk and partition-based assays.

### BACKGROUND

Microfluidic technologies have been developed for introducing individual biological samples (e.g., cells) into discrete partitions (e.g., droplets). Each partition may be fluidically isolated from other partitions, enabling accurate control of respective environments in each discrete partition, and thereby allowing for the biological sample in each partition to be processed separately. For example, the biological samples in each partition can be barcoded and subjected to chemical or physical processes such as heating, cooling, or chemical reactions. This allows the biological sample in each discrete partition to be qualitatively or quantitatively processed in its own separate partition-based assay.

Biological samples containing a variety of biomolecules can be processed in partitioned-based assays for various purposes, such as detection of a disease (e.g., cancer) or genotyping (e.g., species identification). Biological samples, however, are unstable when removed from their viable biological niche, and their physical decomposition begins immediately. The rate and degree of decomposition is determined by a number of factors including time, solution buffering conditions, temperature, source (e.g. certain tissues and cells a have higher levels of endogenous RNase activity), biological stress (e.g. enzymatic tissue dissociation can activate stress response genes), and physical manipulation (e.g. pipetting, centrifuging). The degradation affects important nucleic acid molecules (e.g., RNA), proteins, as well as the higher-order 3D structure of molecular complexes, whole cells, tissues, organs, and organisms. The instability of biological samples is a significant obstacle for their use with partition-based assays (e.g., single-cell assays). Sample degradation greatly limits the ability to use such assays accurately and reproducibly with a wide range of available biological samples.

The problem of biological sample instability is compounded when using tissue samples, which typically include a variety of cell types with differing characteristics. The ability to fix and preserve the different cell types present in tissue samples can vary greatly depending on their particular structure, function, and spatial location within the tissue. Standard biological methods for preparing samples of the different cell types from tissue involve dissociating the tissue with heat and enzymes, however, these methods can greatly alter or destroy the native state of certain cell types. The ability to obtain accurate measurements from cells of differing types found in tissue in partition-based, single-cell assays, requires that the different cell types are rapidly fixed and dissociated so that the relevant assay can be carried out before sample degradation occurs. The use of standard preservatives and fixatives with tissue samples, however, results in measurements highly skewed due to the inability of these standard techniques to reach all cell types of a tissue sample in a manner that accurately preserves their native state.

### SUMMARY

The present disclosure provides methods and devices for biological sample preparation and analysis that use a chopping and fixing treatment in the preparation of dissociated fixed cells from biological tissue for use in either bulk assays, or in single-cell/single-nucleus assays, such as partition-based assays of nucleic acid sequences present in a cell.

In at least one embodiment, the present disclosure provides method for preparing a biological sample, wherein the method comprises:
(a) chopping a biological tissue into a composition of tissue fragments;
(b) treating the composition of tissue fragments with a fixing reagent, thereby providing a composition of fixed tissue fragments; and
(c) treating the composition of fixed tissue fragments with a cell dissociation reagent, thereby providing a composition of fixed cells or fixed nuclei.

In at least one embodiment of the method for preparing a biological sample, the method further comprises: (d) treating the composition of fixed cells or nuclei with a solution comprising an un-fixing agent, thereby providing un-fixed cells or nuclei of a plurality of types from the biological tissue.

In at least one embodiment, the present disclosure also provides a method for analysis of a biological tissue, wherein the method comprises:
(a) chopping a biological tissue to provide a composition of tissue fragments;
(b) treating the composition of tissue fragments with a solution comprising a fixation agent to provide a composition of fixed tissue fragments;
(c) treating the composition of fixed tissue fragments with a cell dissociation reagent to provide a composition of fixed cells or nuclei, each fixed cell or nucleus comprising a plurality of crosslinked nucleic acid molecules; and
(d) generating a plurality of barcoded nucleic acid molecules from said plurality of crosslinked nucleic acid molecules and a plurality of nucleic acid barcode molecules, wherein a barcoded nucleic acid molecule of said plurality of barcoded nucleic acid molecules comprises i) a sequence corresponding to a crosslinked nucleic acid molecule of said plurality of crosslinked nucleic acid molecules or a complement thereof, and ii) a barcode sequence or a complement thereof.

In at least one embodiment, the present disclosure also provides a method for analysis of a biological tissue, wherein the method comprises:
(a) chopping a biological tissue to provide a composition of tissue fragments;
(b) treating the composition of tissue fragments with a solution comprising a fixation agent to provide a composition of fixed tissue fragments;
(c) treating the composition of fixed tissue fragments with a cell dissociation reagent to provide a composition of fixed cells or nuclei, each fixed cell or nucleus comprising a plurality of crosslinked nucleic acid molecules;
(d) treating the composition of fixed cells or nuclei with an un-fixing agent to provide a composition of un-fixed cells or nuclei, each un-fixed cell or nucleus comprising a plurality of un-crosslinked nucleic acid molecules; and
(e) generating a plurality of barcoded nucleic acid molecules from said plurality of un-crosslinked nucleic acid molecules and a plurality of nucleic acid barcode molecules, wherein a barcoded nucleic acid molecule of said plurality of barcoded nucleic acid molecules comprises i) a sequence corresponding to an un-crosslinked nucleic acid molecule of said plurality of un-crosslinked nucleic acid molecules or a complement thereof, and ii) a barcode sequence or a complement thereof.

In at least one embodiment of the methods for analysis of a biological tissue, said generating a plurality of barcoded nucleic acid molecules is performed in a plurality of partitions; optionally, wherein said plurality of partitions is a plurality of droplets or wells. In at least one embodiment, the partition of said plurality of partitions comprises a fixed cell or nucleus and a support comprising said plurality of nucleic acid barcode molecules; optionally, wherein said support is a bead. In at least embodiments, said barcode sequence is a partition-specific barcode sequence.

In at least one embodiment of the method for analysis of a biological tissue that comprises treating the composition of fixed cells or nuclei with an un-fixing agent, said treating with said un-fixing agent is performed in a plurality of partitions.

In at least one embodiment of the methods for preparing a biological sample or the methods for analysis of a biological tissue provided by the present disclosure, the composition of tissue fragments comprises particles of an average size on a side of about 500 μm or less, about 250 μm or less, about 125 μm or less, about 75 μm or less, or about 50 μm or less. In at least one embodiment of the methods, the composition of tissue fragments comprises particles of an average size on a side of between about 50 μm and 500 m, between about 125 μm and 500 m, between about 250 μm and 500 m, between about 50 μm and 250 m, or between about 50 m and 125 m.

In at least one embodiment of the methods for preparing a biological sample or the methods for analysis of a biological tissue, the fixing reagent is paraformaldehyde (“PFA”); optionally, wherein the PFA is in a solution at a concentration of 1%-4% PFA.

In at least one embodiment of the methods for preparing a biological sample or the methods for analysis of a biological tissue, the cell dissociation reagent comprises collagenase.

In at least one embodiment of the methods for preparing a biological sample or the methods for analysis of a biological tissue, the amount of time prior to treating the fixed tissue fragments with the cell dissociation reagent is at least 1 hour, at least 2 hours, at least 6 hours, at least 12 hours, at least 24 hours, at least 1 week, at least 1 month, at least 6 months, or longer.

In at least one embodiment of the methods for preparing a biological sample or the methods for analysis of a biological tissue, the composition of fixed cells or nuclei comprises a plurality of fixed cell types or fixed nuclei from a plurality of cell types.

In at least one embodiment of the methods for preparing a biological sample or the methods for analysis of a biological tissue, a fixed cell or nucleus of said composition of fixed cells or nuclei comprises a plurality of crosslinked nucleic acid molecules.

In at least one embodiment of the methods for preparing a biological sample or for analysis of a biological tissue, wherein the method comprises treating the composition of fixed cells or nuclei with an un-fixing agent, the un-fixing agent is capable of removing crosslinks formed in biomolecules by fixation with a paraformaldehyde (“PFA”) solution at a concentration of 1%-4% PFA. In at least one embodiment, the un-fixing agent comprises a compound selected from compound (1), compound (2), compound (3), compound (4), compound (5), compound (6), compound (7), compound (8), compound (9), compound (10), compound (11), compound (12), compound (13), compound (14), compound (15), or a combination thereof. In at least one embodiment, the un-fixing agent is at a concentration of about 1 mM to about 500 mM, about 50 mM to about 300 mM, or about 50 mM to about 200 mM.

In at least one embodiment of the methods for preparing a biological sample or for analysis of a biological tissue, wherein the method comprises treating the composition of fixed cells or nuclei with an un-fixing agent, the solution comprising the un-fixing agent further comprises a protease. In at least one embodiment, the protease is a thermolabile protease or cold-active protease; optionally, wherein the protease is selected from Subtilisin A, Proteinase K, ArcticZymes Proteinase, and a combination thereof.

In at least one embodiment of the methods for preparing a biological sample or for analysis of a biological tissue, the biological tissue is selected from brain tissue, skin tissue, muscle tissue, smooth muscle tissue, cardiac muscle tissue, skeletal muscle tissue, bone marrow tissue, lung tissue, bronchial tissue, fallopian tube tissue, gallbladder tissue, ovarian tissue, testicular tissue, hypothalamus tissue, thyroid tissue, adrenal gland tissue, kidney tissue, pancreatic tissue, small intestine tissue, large intestine tissue, colon tissue, liver tissue, lymphatic tissue, breast tissue, mammary gland tissue, mesenteric tissue, nasal tissue, pineal gland tissue, parathyroid tissue, pharynx tissue, larynx tissue, pituitary tissue, prostate tissue, salivary tissue, spinal cord tissue, spleen tissue, stomach tissue, thymic tissue, tracheal tissue, tongue tissue, urethral tissue, placental tissue, arterial tissue, venous tissue, and tonsil tissue.

In at least one embodiment of the methods for preparing a biological sample or for analysis of a biological tissue, the method can further comprise filtering and/or sieving the composition of fixed cells or nuclei and/or the un-fixed cells or nuclei.

In at least one embodiment, the present disclosure provides a chopping device for mechanically chopping a biological tissue sample, the device comprising:
(a) a razor blade holder comprising a plurality of razor blade slots, each slot configured to insert a cutting edge of a razor blade through the razor blade slot and hold a top portion of the razor blade within the razor blade slot, wherein a bottom portion of the razor blade including the razor blade cutting edge extends vertically below the razor blade holder;
(b) a razor blade alignment layer comprising a plurality of alignment openings, wherein each alignment opening is configured to insert the cutting edge of the razor blade through the alignment layer so that the bottom portion of the razor blade including the razor blade cutting edge extends below the alignment layer; and
(c) a circular specimen dish holder configured to hold a specimen dish containing a biological tissue sample;
wherein the razor blade holder is configured to be placed on top of and connected to the razor blade alignment layer so that each of the plurality of razor blade slots are aligned above each of the corresponding plurality of alignment openings; and
wherein the razor blade alignment layer is configured to be placed on top of and reversibly and rotatably connected to the circular specimen dish holder so that each razor blade cutting edge extends into and toward the bottom of the circular specimen dish holder.

In at least one embodiment of the chopping device, the razor blade holder comprises at least two through-holes and at least two screws inserted through the at least two through-holes for reversibly connecting the razor blade holder onto the razor blade alignment layer. In at least one embodiment, each of the least two screws is a spring-loaded screw configured to provide a vertical lift force to the razor blade holder relative to the alignment layer to provide a collapsible gap between the razor blade holder and alignment layer. In at least one embodiment, the spring-loaded screws are configured so the gap between the bottom of the razor blade holder and the top of the alignment layer ranges from 0.5 to 10 mm, at rest.

In at least one embodiment of the chopping device, the device further comprises a handle connected to the top of the razor blade holder; optionally, wherein the handle comprises at least one through-hole for a screw for connecting the handle to the razor blade holder.

In at least one embodiment of the chopping device: (i) the razor blade holder has a holder diameter and the plurality razor blade slots are parallel and have a length of at least 85% of the holder diameter; and/or (ii) the razor blade alignment layer has an alignment layer diameter and the plurality alignment openings are parallel and have a length of at least 75% of the holder diameter.

In at least one embodiment of the chopping device, the razor blade holder and the razor blade alignment layer are configured so that the cutting edge of each razor blade inserted into the slots and openings will contact the bottom of the specimen dish when the razor blade holder and the razor blade alignment layer are pushed down.

In at least one embodiment of the chopping device, the circular specimen dish holder comprises a top circular rim and the alignment layer comprises an annular channel around the outer perimeter of the circular bottom surface and corresponding to the top circular rim of the specimen dish holder, wherein the annular channel is configured to receive the top circular rim to reversibly and rotatably connect the alignment layer onto the circular specimen dish holder.

In at least one embodiment of the chopping device, the alignment layer comprises a second annular channel around the outer perimeter of the circular bottom surface and within the inner circumference of the annular channel, wherein the second annular channel is configured to receive a top circular rim of a specimen dish held within the circular specimen dish holder.

In at least one embodiment, the present disclosure also provides a method of using a chopping device, wherein the device comprises a plurality of razor blades inserted into the plurality of razor blade slots and the plurality of alignment openings and placed on top of and reversibly and rotatably connected to the circular specimen dish holder containing an open circular specimen dish containing a tissue sample, the method comprising:
(i) pressing the razor blade holder down thereby forcing the cutting edges of the plurality of razor blades onto and cutting the tissue sample contained within the specimen dish;
(ii) rotating the plurality of razor blades relative to the tissue sample to a second orientation; and
(iii) pressing the razor blade holder down thereby forcing the cutting edges of the plurality of razor blades onto and cutting the tissue sample at the second orientation.

In at least one embodiment, the method of using the chopping device further comprises rotating the plurality of razor blades relative to the tissue sample to a third orientation and pressing the razor blade holder down on the circular specimen dish holder thereby forcing the cutting edges of the plurality of razor blades onto the tissue sample and cutting tissue sample at the third orientation; and optionally, the method further comprises rotating the plurality of razor blades relative to the tissue sample to a fourth orientation and pressing the razor blade holder down on the circular specimen dish holder thereby forcing the cutting edges of the plurality of razor blades onto the and cutting tissue sample at the fourth orientation. Optionally, in the method, the plurality of razor blades is rotated at least 10-25 degrees each rotation.

In at least one embodiment, the present disclosure provides a kit for preparing a biological sample, wherein the kit comprises: a fixing reagent, a cell dissociation reagent, and assay reagents. In at least one embodiment, the kit further comprises an un-fixing agent. In at least one embodiment, the kit further comprises a chopping device. In at least one embodiment, the present disclosure provides a kit containing, in one or more containers, the chopping device of the present disclosure and one or more razor blades.

### BRIEF DESCRIPTION OF THE FIGURES

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows an example of a microfluidic channel structure for partitioning individual biological particles.

FIG. 2 shows an example of a microfluidic channel structure for delivering barcode carrying beads to droplets.

FIG. 3 shows an example of a microfluidic channel structure for co-partitioning biological particles and reagents.

FIG. 4 shows an example of a microfluidic channel structure for the controlled partitioning of beads into discrete droplets.

FIG. 5 shows an example of a microfluidic channel structure for increased droplet generation throughput.

FIG. 6 shows another example of a microfluidic channel structure for increased droplet generation throughput.

FIG. 7 shows an exemplary barcode carrying bead.

FIG. 8 shows another exemplary barcode carrying bead.

FIG. 9 shows an exemplary microwell array schematic.

FIG. 10 shows an exemplary microwell array workflow for processing nucleic acid molecules.

FIG. 11 schematically illustrates examples of labelling agents.

FIG. 12 depicts an example of a barcode carrying bead.

FIG. 13A, FIG. 13B, and FIG. 13C schematically depict an example workflow for processing nucleic acid molecules.

FIG. 14A, FIG. 14B, and FIG. 14C depicts comparative plots of percentage of total genes detected and total UMIs detected in fixed samples relative to fresh control samples prepared and analyzed as described in Example 2. FIG. 14A depicts plots of results for “dissociated then fixed” adult mouse kidney tissue at days 3 and 7 post-fixation; FIG. 14B depicts plots of results for “chop-fixed” adult mouse kidney tissue at days 1 and 6 post-fixation; and FIG. 14C depicts plots of results for “dissociated then fixed” adult mouse brain tissue at days 1 and 6 post-fixation.

FIG. 15A and FIG. 15B depict comparative plots ATAC transposition events from chop-fixed E18 brain tissue nuclei samples prepared as described in Example 4.

FIG. 16 depicts comparative plots of cell-type clusters detected in kidney tissue samples that were chop-fixed, dissociated, un-fixed, and stored for up to 6 days as described in Example 5.

FIG. 17 illustrates a transposase-nucleic acid complex comprising a transposase, a first double-stranded oligonucleotide comprising a transposon end sequence and a first primer sequence and a second double-stranded oligonucleotide comprising a transposon end sequence and a second primer sequence.

FIG. 18 illustrates a transposase-nucleic acid complex comprising a transposase, a first double-stranded oligonucleotide comprising a transposon end sequence and first and second primer sequences and a second double-stranded oligonucleotide comprising a transposon end sequence and third and fourth primer sequences.

FIG. 19 illustrates a transposase-nucleic acid complex comprising a transposase, a first hairpin molecule, and a second hairpin molecule

FIG. 20 illustrates a schematic workflow for tandem ATAC and RNA processing.

FIG. 21 depicts a side view perspective illustration of an exploded view of an embodiment of a mechanical chopping device of the present disclosure, including a specimen dish.

FIG. 22 depicts a top view illustration of the chopping device system of FIG. 21 after assembly.

FIG. 23A depicts a side view illustration of an embodiment of a mechanical chopping device of the present disclosure in a resting or normal position. FIG. 23B is a side view of the chopping device of FIG. 23A in a compressed slicing position. FIG. 23C is a perspective side view of the chopping device of FIG. 23A in a resting or normal position. FIG. 23D is a perspective side view of the chopping device of FIG. 23A in compressed position.

FIG. 24A is a side view illustration of a chopping device according to another embodiment in resting or normal position. FIG. 24B is a side cross-sectional view of the chopping device of FIG. 24A in compressed position.

FIG. 25 is a side view cross-sectional illustration of a chopping device according to another embodiment in resting or normal position (“rest mode”).

FIG. 26 is side view illustration of an exploded non-assembled chopping device system including a specimen dish and razor blades according to another embodiment of the invention.

FIG. 27A is a top view illustration of an alignment layer component of a chopping device according to one preferred embodiment of the invention. FIG. 27B is a cross-sectional side view along line A-A of FIG. 27A. FIG. 27C is a side view of FIG. 27A. FIG. 27D is a bottom view of FIG. 27A.

FIG. 28A is a top perspective illustration of an alignment layer component according to another embodiment of the invention. FIG. 28B is a bottom perspective illustration of the alignment layer component of FIG. 28A.

FIG. 29A, FIG. 29B, FIG. 29C, FIG. 29D, FIG. 29E, and FIG. 29F depict comparative plots of cell-type clusters detected in human uterine tissue samples that were dissociated, with or without first being chop-fixed, and/or flash-frozen, and/or stored for up to 5 days as described in Example 7.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of”.

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of these limits, ranges excluding (i) either or (ii) both of those included limits are also included in the disclosure. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present disclosure pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### A. Overview of Chop-Fix Methods

Recognized herein is the need for methods, compositions, kits, and systems for analyzing multiple cellular analytes (e.g., genomic, epigenomic, transcriptomic, metabolomic, and/or proteomic information) present in biological samples obtained from tissue. The ability to carry out an accurate assay of a biological sample from tissue requires the rapid and efficient release of the wide range of cell types from the tissue so that the relevant cellular analyte information (e.g., RNA transcripts) within each of the various cell types can be obtained before degradation occurs. Ideally, the state of the cellular analytes released from the various cell types in tissue is not significantly altered relative to its natural environment, that is the state it is in the cell within the tissue matrix before the treatment to release it. Typical methods for releasing cellular analytes from a biological tissue sample for use in an assay involves the use of some combination of lysis agents, enzymatic inhibitors, chelating agents, physical agitation, and heat to facilitate the activity of the various reagents involved.

For example, it is widely recognized by practitioners of single-cell assays for transcriptome profiling that the exposure of whole unfixed tissue samples to room temperature conditions (e.g., after removing from on-ice conditions) results in the rapid degradation of the RNA transcripts present within the tissue. There are two main reasons for rapid degradation of RNA transcripts during tissue sample preparation leading up to analysis. First, RNA is made up of ribose units with a highly reactive C2 hydroxyl group that makes RNA more chemically labile than DNA and also more prone to heat degradation. Second, enzymes that degrade RNA (e.g., RNases) are ubiquitous and removing them prior to or during sample preparation is nearly impossible. Additionally, damaged and decaying eukaryotic tissue is known to produce more RNases. Thus, human tissue that is subjected to surgical extraction and allowed to sit at room temperature is very difficult to work with in a workflow that utilizes single-cell assaying of RNA transcripts.

The present disclosure provides methods that result in improved release of cellular analytes from biological tissue samples that allow for the analysis of the various populations of different cell types that are present in the natural environment of such tissue. The biological samples prepared using these methods allow improved assays, including partition-based assays, to be carried out using fixed cells or nuclei from such tissue samples and fewer or no artifacts in the cellular analyte information that is obtained. Standard methods known in the art prepare samples for partition-based analysis by treating whole tissue samples with a dissociation reagent or a fixing reagent to provide isolated cells for analysis. These methods however result in samples that provide cellular analyte measurements that differ in significant ways from fresh samples. Notably, certain rare cell types found in freshly prepared cells from tissue samples are not detected. It is surprising advantage of the present disclosure that the methods of the disclosed herein that utilize chopping and fixing of tissue samples provide biological samples for partition-based analysis that retain the cellular analytes from rare cell types found in tissue (e.g., neuronal cell types from brain tissue). The chopping and fixing treatment disclosed herein are able to provide samples of cells or nuclei from tissue for use in either bulk assays, or in single-cell/single-nucleus assays, such as partition-based assays of nucleic acid sequences present in a cell or in a nucleus.

The general method for preparation of a biological sample provided by the present disclosure comprises: (a) chopping a biological tissue into a composition of tissue fragments; (b) treating the composition of tissue fragments with a fixing reagent, thereby providing a composition of fixed tissue fragments; and (c) treating the composition of fixed tissue fragments with a cell dissociation reagent. This chop-fix treatment together with the cell dissociation reagent provides a composition of fixed cells or nuclei from the tissue that includes a distribution of the various cell types from the tissue that can then be used for further bulk or partition-based analysis of cellular analytes. Such methods for bulk or partition-based analysis of cells or nuclei (e.g., single-cell/single-nucleus analysis of DNA and/or RNA sequences) useful with the chop-fix methods of biological sample preparation are provided elsewhere herein.

The “biological tissue” used in the methods of the present disclosure can any tissue of biological origin, including solid tissue samples from a tissue specimen, a biopsy, or tissue from a tissue culture. The tissue sample can includes samples that have been manipulated after isolation from the biological source, such as by treatment with reagents, or samples of tissues that were embedded in a medium (e.g., paraffin), sectioned tissue sample (e.g., sectioned samples that are mounted on a solid substrate such as a glass slide), washed tissue, and/or tissue enriched for certain cell populations (such as cancer cells, neurons, stem cells, etc.), tissue obtained by surgical resection, tissue obtained by biopsy, tissue samples from organs, bone marrow, blood, plasma, serum, and the like. It is contemplated that the biological tissue used in the methods of the present disclosure can be derived from another sample. Biological tissue samples can include tissue obtained core biopsy, needle aspirate, or fine needle aspirate.

Biological tissues useful with the methods of the present disclosure include: brain tissue, skin tissue, muscle tissue, smooth muscle tissue, cardiac muscle tissue, skeletal muscle tissue, bone marrow tissue, lung tissue, bronchial tissue, fallopian tube tissue, gallbladder tissue, ovarian tissue, testicular tissue, hypothalamus tissue, thyroid tissue, adrenal gland tissue, kidney tissue, pancreatic tissue, small intestine tissue, large intestine tissue, colon tissue, liver tissue, lymphatic tissue, breast tissue, mammary gland tissue, mesenteric tissue, nasal tissue, pineal gland tissue, parathyroid tissue, pharynx tissue, larynx tissue, pituitary tissue, prostate tissue, salivary tissue, spinal cord tissue, spleen tissue, stomach tissue, thymic tissue, tracheal tissue, tongue tissue, urethral tissue, placental tissue, arterial tissue, venous tissue, and tonsil tissue. Additionally, it is contemplated that biological tissues useful with the methods of the present disclosure can include a cancer of any one or more of the aforementioned biological tissue types.

The “biological sample,” prepared using the method can include a cell, a biomolecule, such as a nucleic acid, a protein, a carbohydrate, a lipid, and/or compositions of any combination thereof. The biological sample may include cells and other biological materials derived from tissue and/or cell, or be a cell-free sample. A cell-free sample may include extracellular polynucleotides.

The term “chopping,” as used herein, refers to any mechanical activity of separating a larger piece of a material (e.g., a biological tissue) into multiple smaller pieces or fragments. Simply treating whole tissue samples with fixing reagents and/or dissociation reagents fails to result sample of fixed cells or nuclei from the tissue that are provide results for cellular analytes in bulk or partition-based assays that are representative of the fresh tissue. A range of mechanical methods and devices for chopping tissue are available in the art and can be used in the methods of the present disclosure. Exemplary chopping methods and devices are provided elsewhere herein including the examples.

The term “tissue fragments” refers to a piece resulting from chopping a tissue, and is intended to include pieces that are a mixture of the cells and other biological materials that are characteristic of the type of tissue. A tissue piece can include a plurality of cell types found characteristic of the tissue type. Without intending to be bound by theory or a particular mechanism, it is believed that the chopping provides a composition of tissue fragments that are able to achieve rapid fixation and then dissociation without certain biological stresses that skew the distribution and/or representation of cell types and other cellular analytes from tissue samples.

As disclosed elsewhere herein, the chopping of the biological tissue is carried out to provide a composition of tissue fragments comprising particles of an average size on a side of about 500 μm or less, about 250 μm or less, about 125 μm or less, about 75 μm or less, or about 50 μm or less. Generally, useful ranges of the composition of tissue fragments comprises particles of an average size on a side of between about 50 μm and 500 m, between about 125 m and 500 m, between about 250 μm and 500 m, between about 50 μm and 250 m, or between about 50 μm and 125 m.

Additionally, as disclosed elsewhere herein, the chopping of the biological tissue is carried out near ice-cold temperatures (e.g., between about 2C to 8C) on a smooth, flat surface, with an chopping action that minimizes stress on the various cell types present in the tissue. For example, a fresh brain tissue sample is placed in a clean petri dish kept on ice and a single clean razor blade is used to finely chop by hand the tissue with a step size between each cut kept as small possible. The chopping is carried out with no, or minimal, dragging or swirling of the tissue. After chopping across one entire dimension of brain tissue sample on the petri dish surface, the chopping is repeated across at least 2 additional dimensional axes of the sample in the same manner. This chopping process results in optimally sized tissue fragments for rapid fixation and further dissociation with minimal heat or mechanically induced stress resulting in degradation of cellular contents. It is also contemplated that this chopping can be carried using a mechanical device that mimics the hand-chopping method including the spacing and directionality along three distinct axes across the sample.

In at least one embodiment, the chopping of the biological tissue for use in the chop-fix method of the present disclosure can be carried out using a mechanical “chopping device” (or “tissue slicer device”) as described in greater detail elsewhere herein, and in the US provisional patent application entitled “Tissue Slicer Devices And Methods Of Using The Same,” having U.S. Ser. No. 63/213,908, and filed on Jun. 23, 2021, which is hereby incorporated by reference herein in its entirety.

The chopping process results in tissue fragments that can be immediately fixed by immersion in a fixative or fixing reagent solution. “Fixed” refers to the state of being preserved from decay and/or degradation. Without intending to be bound by a specific theory or mechanism, generally the fixation process is the result of biomolecules within the sample contacting the fixation reagent for some amount of time, whereby crosslinks between the biomolecules in the sample are formed that prevent degradative processes. The chopped tissue is immersed in the fixing reagent solution typically overnight at about 4 C. The “fixed tissue fragments” include the fragments of tissue resulting from chopping that undergo fixation in their component cells and cellular analytes to at least some extent due to this treatment with a fixing reagent.

For example, an exemplary process for preparing the fixed tissue fragments from chopped brain tissue includes immediately immersing the chopped tissue in a 1%-4% solution of the fixing reagent, paraformaldehyde (PFA) and then stored in a tube on its side overnight at 4 C. Typically, the ratio of the volume of fixing reagent solution to the volume of tissue fragments is about 10:1. In some embodiments, the ratio of the volume of fixing reagent solution to the volume of tissue fragments can be at least about 2.5:1, at least about 5:1, at least about 7.5:1, at least about 15:1, or at least about 20:1. After the overnight storage, the tube is spun down (e.g., at 500 rcf for 5 minutes) and the solution supernatant removed to provide fixed tissue fragments. The fixed tissue fragments can be stored with minimal degradation for varying amounts of time before further dissociation and/or analysis (e.g., in a partition-based assay). In at least one embodiment, the fixed tissue fragments can be stored for at least 1 hour, at least 2 hours, at least 6 hours, at least 12 hours, at least 24 hours, at least 1 week, at least 1 month, at least 6 months, or longer, before they are used.

Typically, the fixed tissue fragments are resuspended with a cell resuspension buffer (e.g., 1% BSA+0.2 U/mL RNase inhibitor in PBS) before cell dissociation. “Cell dissociation” refers to a process of treating a larger solid composition that includes a plurality of cells and other biological material (e.g., a fixed tissue fragment) so that it dissociates into individual cells. In the context of the methods of the present disclosure, it is contemplated that cell dissociation of the fixed tissue fragments can be carried out using any cell dissociation reagent solution known in the art. Because tissue fragments comprise cells associated by biological material that includes the protein collagen, in at least one embodiment, the cell dissociation reagent used to treat the fixed tissue fragments comprises collagenase. For example, as disclosed elsewhere herein, the buffer in which fixed brain tissue fragments are resuspended is removed and replaced with the dissociation reagent, Collagenase A. A tube of this cell dissociation reagent mixture with the fixed tissue fragments is then shaken (e.g., at ˜700 rpm for ˜90 min) at a temperature of 37 C. Additionally, dissociation of cells in the fixed tissue fragments can be facilitated through other physical means, for example repeated pipetting and spinning down.

B. Use of Cells or Cell Nuclei from Chop-Fix Methods in Assays

The product of the chop-fix method for preparing biological samples is a preparation of dissociated fixed cells derived from tissue samples or fixed nuclei derived from tissue samples that can be further treated and/or used in various methods of analysis. The ability to use a fixed cells or nuclei in an assay requires rapid and efficient preparation so that the relevant cellular analytes are fixed before degradation occurs. Ideally, the assay data obtained from the fixed cells or nuclei should resemble a fresh sample obtained from its natural environment as closely as possible. The methods for chop-fix preparation of tissue samples of the present disclosure provide dissociated fixed cells or fixed nuclei from tissue that can be used in a range of assays.

Generally, each fixed cell or nucleus generated by the method comprises a plurality of crosslinked nucleic acid molecules as a result of the treatment with the fixative. These crosslinks act to prevent degradation of nucleic acid molecules, particularly RNA transcripts, and thereby preserve the cellular information associated with populations of specific nucleic acid molecules in their native or fresh state. As described elsewhere herein, it is contemplated that assays directed to detecting and/or measuring the populations of nucleic acid sequences present in the fixed cells or nuclei can be carried out using compositions of fixed cells or nuclei from tissue samples prepared using the chop-fix method. For example, in at least one embodiment, present disclosure provides a method for analysis of a biological tissue, wherein the method comprises: (a) chopping a biological tissue to provide a composition of tissue fragments; (b) treating the composition of tissue fragments with a solution comprising a fixation agent to provide a composition of fixed tissue fragments; (c) treating the composition of fixed tissue fragments with a cell dissociation reagent to provide a composition of fixed cells, each fixed cell or nucleus comprising a plurality of crosslinked nucleic acid molecules; and (d) generating a plurality of barcoded nucleic acid molecules from said plurality of crosslinked nucleic acid molecules and a plurality of nucleic acid barcode molecules, wherein a barcoded nucleic acid molecule of said plurality of barcoded nucleic acid molecules comprises i) a sequence corresponding to a crosslinked nucleic acid molecule of said plurality of crosslinked nucleic acid molecules or a complement thereof, and ii) a barcode sequence or a complement thereof. This general method of generating barcoded nucleic acid molecules from the crosslinked nucleic acids present in the fixed cell or fixed nucleus compositions can be carried using a variety of partition-based assay methods that are described in greater detail in elsewhere herein, including the examples.

The ability to prepare a biological sample of dissociated fixed cells or fixed nuclei for use in an assay starting from a tissue sample is a feature of the methods of the present disclosure. A “fixed biological sample” refers to a biological sample that has been contacted with a fixation reagent. For example, a formaldehyde-fixed biological sample has been contacted with the fixation reagent formaldehyde. “Fixed cells”, “fixed nuclei” or “fixed tissues” refer to cells, nuclei or tissues that have been in contact with a fixative under conditions sufficient to allow or result in the formation of intra- and inter-molecular covalent crosslinks between biomolecules in the biological sample.

The amount of time a biological sample is contacted with a fixative to provide a fixed biological sample depend on the temperature, the nature of the sample, and the fixative used. For example, a biological sample can be contacted by a fixation reagent for 72 or less hours (e.g., 48 or less hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes).

Generally, contact of biological sample (e.g., a tissue fragment) with a fixation reagent (e.g., paraformaldehyde or PFA) results the formation of intra- and inter-molecular covalent crosslinks between biomolecules in the biological sample. In some cases, the fixation reagent, formaldehyde, is known to result in covalent aminal crosslinks within RNA, DNA, and/or protein molecules. Examples of fixation reagents include but are not limited to aldehyde fixatives (e.g., formaldehyde, also commonly referred to as “paraformaldehyde,” “PFA,” and “formalin”; glutaraldehyde; etc.), imidoesters, NHS (N-Hydroxysuccinimide) esters, and the like.

The formation of crosslinks in biomolecules (e.g., proteins, RNA, DNA) due to fixation can affect the ability to detect (e.g., bind to, amplify, sequence, hybridize to) the biomolecules in standard assay methods, and methods for removing or un-fixing crosslinks are described elsewhere herein. Some assays, however, can detect and provide useful information from fixed cells or nuclei. The widely used fixative reagent, paraformaldehyde or PFA, fixes tissue samples by catalyzing crosslink formation between basic amino acids in proteins, such as lysine and glutamine. Both intra-molecular and inter-molecular crosslinks can form in the protein. These crosslinks can preserve protein secondary structure and also eliminate enzymatic activity in the preserved tissue sample.

The present disclosure provides methods, and kits for preparing biological samples of fixed cells or nuclei from biological tissue, and these fixed cells or nuclei can be used in assays to detect and/or measure a range of cellular analytes. Suitable cellular analytes include, without limitation, intracellular/intranuclear and extracellular analytes. The cellular analyte may be a protein, a metabolite, a metabolic byproduct, an antibody or antibody fragment, an enzyme, an antigen, a carbohydrate, a lipid, a macromolecule, or a combination thereof (e.g., proteoglycan) or another biomolecule. The cellular analyte may be a nucleic acid molecule. The cellular analyte may be a deoxyribonucleic acid (DNA) molecule or a ribonucleic acid (RNA) molecule. The DNA molecule may be a genomic DNA molecule. The cellular analyte may comprise coding or non-coding RNA. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA.

In some instances, the cellular analyte is associated with an intermediary entity, wherein the intermediary entity is analyzed to provide information about the cellular analyte and/or the intermediary entity itself. For instance, an intermediary entity (e.g., an antibody) may be bound to an extracellular analyte (e.g., a cell surface receptor) or a nuclear membrane protein, where the intermediary entity is processed to provide information about the intermediary entity, the extracellular analyte, or both. In one embodiment, the intermediary entity comprises an identifier (e.g., a barcode molecule) that can be used to generate barcode molecules (e.g., droplet-based barcoding) as further described herein.

In some embodiments, the fixed cells or nuclei resulting from the chop-fix methods are fixed by treatment with formaldehyde. The term “formaldehyde” when used in the context of a fixative also refers “paraformaldehyde” (or “PFA”) and “formalin”, both of which are terms with specific meanings related to the formaldehyde composition (e.g., formalin is a mixture of formaldehyde and methanol). Thus, a formaldehyde-fixed tissue fragment, or cell/cell nucleus may also be referred to as formalin-fixed or PFA-fixed. Protocols and methods for the use of formaldehyde as a fixation reagent to prepare fixed biological samples are well known in the art, and can be used in the methods of the present disclosure. For example, suitable ranges of formaldehyde concentrations for use in preparing a fixed biological sample is 0.1 to 10%, 1-8%, 1-4%, 1-2%, 3-5%, or 3.5-4.5%. In at least one embodiment of the chop-fix method of the present disclosure, the tissue fragments are fixed using a final concentration of 1% formaldehyde, 4% formaldehyde, or 10% formaldehyde. Typically, the formaldehyde is diluted from a more concentrated stock solution—e.g., a 35%, 25%, 15%, 10%, 5% PFA stock solution.

In at least one embodiment, the methods for preparing a biological sample using the chop-fix treatment of biological tissue use PFA as a fixative, and the stabilizing effect of the fixation allows for the amount of time after fixation prior to dissociating the fixed cells or nuclei from the tissue, and/or further assaying the fixed cells or nuclei to be at least 1 hour, at least 2 hours, at least 6 hours, at least 12 hours, at least 24 hours, at least 1 week, at least 1 month, at least 6 months, or longer.

### C. Un-Fixing Treatments

In some assay methods, it may be useful to use cells or nuclei that do not include the crosslinks caused by fixation. In such methods it may be preferable to prepare un-fixed cells or nuclei. “Un-fixed” refers to the processed condition of a cell, cell nucleus, a plurality of cells, a plurality of nuclei, a tissue sample or any other biological sample that is characterized by a prior state of fixation followed by a reversal of the prior state of fixation. An un-fixed cell or cell nucleus is characterized by broken or reversed covalent bonds in the biomolecules of the cell(s)/nuclei or sample, where such covalent bonds were previously formed by treatment with a fixation agent (e.g., paraformaldehyde or PFA). An un-fixed cell or un-fixed nucleus may also be referred to as a “previously fixed” cell or nucleus.

The present disclosure contemplates that dissociated fixed cells or nuclei obtained from chop-fixed tissue samples can be further treated to provide un-fixed cells or nuclei. Accordingly, in at least one embodiment, the chop-fix method can further comprise treating the composition of dissociated fixed cells or nuclei from the tissue sample with a solution comprising an un-fixing agent.

The term “un-fixing agent” (or “de-crosslinking agent”) as used herein refers to a compound or composition that reverses fixation and/or removes the crosslinks within or between biomolecules in a sample caused by previous use of a fixation reagent. In some embodiments, un-fixing agents are compounds that act catalytically in removing crosslinks in a fixed sample. Exemplary compounds (1)-(15) useful as un-fixing agents in the methods of the present disclosure include the compounds of Table 1 below. Additionally, methods for the preparation and use of un-fixing agents, such as compounds (1)-(15), to prepare biological samples from fixed cells for bulk and partition-based assays of cellular analytes are disclosed in International Patent Application no. PCT/US2020/066701, filed Dec. 22, 2020, which is hereby incorporated by reference herein.

TABLE 1
Exemplary Un-fixing Agent Compounds

| | |
|---|---|
| 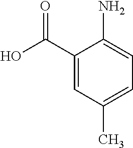 | (1) |
| 2-amino-5-methylbenzoic acid | |
| (CAS No. 2941-78-8; Sigma-Aldrich) | |
| 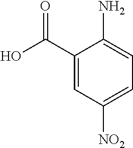 [SMILES: Nc1ccc([N+](=O)[O-])cc1C(=O)O] | (2) |
| 2-amino-5-nitrobenzoic acid | |
| (CAS No. 616-79-5; Sigma-Aldrich) | |
| 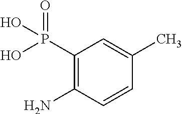 | (3) |
| (2-amino-5-methylphenyl)phosphonic acid | |
| (CAS 69675-98-5; Ambeed Inc.) | |
| 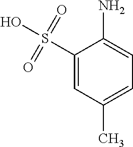 | (4) |
| 2-amino-5-methylbenzenesulfonic acid | |
| (CAS No. 88-44-8; Sigma-Aldrich) | |
| 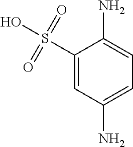 [SMILES: Nc1ccc(N)c(S(=O)(=O)O)c1] | (5) |
| 2,5-diaminobenzenesulfonic acid | |
| (CAS No. 88-45-9; Sigma-Aldrich) | |
| 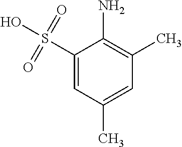 | (6) |
| 2-amino-3,5-dimethylbenzenesulfonic acid | |
| (CAS No. 88-22-2; TCI Co. Ltd., Tokyo, JP) | |
| 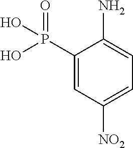 [SMILES: Nc1ccc([N+](=O)[O-])cc1P(=O)(O)O] | (7) |
| (2-amino-5-nitrophenyl)phosphonic acid | |
| 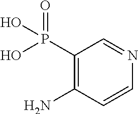 [SMILES: Nc1ccncc1P(=O)(O)O] | (8) |
| (4-aminopyridin-3-yl)phosphonic acid | |
| 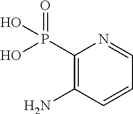 [SMILES: Nc1cccnc1P(=O)(O)O] | (9) |
| (3-aminopyridin-2-yl)phosphonic acid | |
| 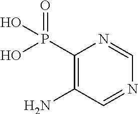 [SMILES: Nc1cncnc1P(=O)(O)O] | (10) |
| (5-aminopyrimidin-4-yl)phosphonic acid | |
| 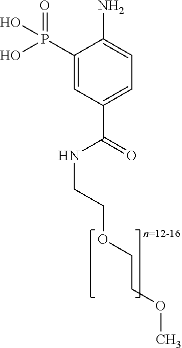 | (11) |
| (2-amino-5-{[2-(2-poly-ethoxy)ethyl] | |
| carbamoyl}phenyl)phosphonic acid | |
| 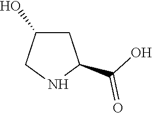 | (12) |
| (2S,4R)-4-hydroxypyrrolidine-2-carboxylic acid | |
| (“trans-4-hydroxy-L-proline;” | |
| CAS No. 51-35-4; Sigma-Aldrich) | |
| 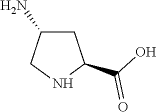 | (13) |
| (2S,4R)-4-aminopyrrolidine-2-carboxylic acid | |
| (“trans-4-aminoproline;” CAS No. 16257-88-8) | |
| 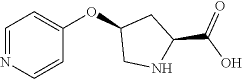 [SMILES: O=C(O)[C@@H]1C[C@H](Oc2ccncc2)CN1] | (14) |
| (2S,4S)-4-[(pyridin-4-yl)oxy]pyrrolidine-2-carboxylic acid | |
| (CAS No. 2309431-82-9; Enamine Ltd.) | |
| 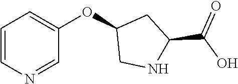 [SMILES: O=C(O)[C@@H]1C[C@H](Oc2cccnc2)CN1] | (15) |
| (2S,4S)-4-[(pyridin-3-yl)oxy]pyrrolidine-2-carboxylic acid | |
| (“cis-m-O-Py-Pro”) | |

At least one of the un-fixing agents of Table 1, compound (3), has previously been shown to catalytically break down the aminal and hemi-aminal adducts that form in RNA treated with formaldehyde, and are compatible with many RNA extraction and detection conditions. See e.g., Karmakar et al., “Organocatalytic removal of formaldehyde adducts from RNA and DNA bases,” Nature Chemistry, 7: 752-758 (2015); and US 2017/0283860A1.

Proline is a unique amino acid that contains a secondary amine in a 5-membered ring, resulting in high nucleophilicity. The high nucleophilicity together with a proximal amine or acid moiety in the proline analog structures of compounds (12), (13), (14), and (15) suggests that these compounds, like the compounds (1)-(11), also can be used as catalytically break down the aminal and hemi-aminal adducts that form in formaldehyde-fixed RNA and other biomolecules.

Compounds (1)-(6), (12), and (14) are commercially available. The compounds (7), (8), (9), (10), (11), (13), and (15) can be prepared from commercially available reagents using standard chemical synthesis techniques well-known in the art. See e.g., Crisalli et al., “Importance of ortho Proton Donors in Catalysis of Hydrazone Formation,” Org. Lett. 2013, 15, 7, 1646-1649.

Compounds (8) can be prepared by a 2-step as described in Example 4. Briefly, in preparing compound (8), the compound, diethyl (4-aminopyridin-3-yl)phosphonate is prepared according to the procedure described in Guilard, R. et al. Synthesis, 2008, 10, 1575-1579. Then, the target compound (8), (4-aminopyridin-3-yl)phosphonic acid) is prepared by acid hydrolysis of the precursor compound of the diethyl (4-aminopyridin-3-yl)phosphonate. Similarly, compounds (9) and (10) can be prepared from straightforward procedures. For example, compound (9) can be prepared in 2-steps from 2-bromopyridin-3-amine (CAS Reg. #39856-58-1; Sigma-Aldrich, St. Louis, MO) as shown in the scheme below.

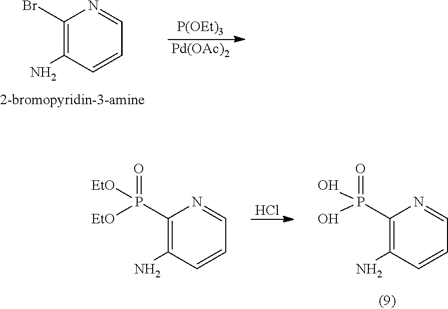 [SMILES: Nc1cccnc1Br ; CCOP(=O)(OCC)c1ncccc1N ; Cl ; Nc1cccnc1P(=O)(O)O ; "Pd(OAc)2" ; "P(OEt)3"]

Compound (10) is prepared similarly in 2-steps from 4-bromopyrimidin-5-amine (CAS Reg. #849353-34-0; Ambeed, Inc., Arlington Heights, IL, USA) as shown in the scheme below.

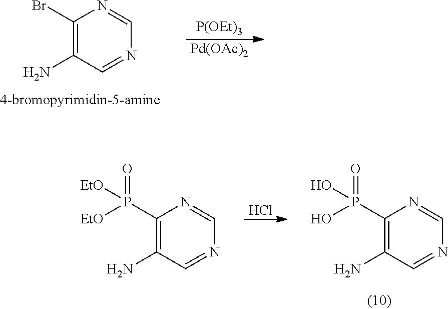 [SMILES: Nc1cncnc1Br ; CCOP(=O)(OCC)c1ncncc1N ; Cl ; Nc1cncnc1P(=O)(O)O ; "Pd(OAc)2" ; "P(OEt)3"]

The proline analog compounds (13) and (15) are prepared via a straightforward single step deprotection from commercially available protected precursor compounds.

Accordingly, in the embodiments of the chop-fix methods of the present disclosure that include a step of un-fixing the fixed cells or nuclei, the un-fixing agent used can comprise a compound selected from Table 1. For example, the un-fixing agent can comprise a compound of any of compound (1), compound (2), compound (3), compound (4), compound (5), compound (6), compound (7), compound (8), compound (9), compound (10), compound (11), compound (12), compound (13), compound (14), compound (15), or a combination of one or more the compounds of Table 1.

In at least one embodiment of the method, the tissue fragments are fixed with PFA and the un-fixing agent used in the solution is capable of removing crosslinks formed in biomolecules by fixation with PFA. In at least one embodiment, the un-fixing agent is a composition comprising a compound selected from compound (1), compound (2), compound (3), compound (4), compound (5), compound (6), compound (7), compound (8), compound (9), compound (10), compound (11), compound (12), compound (13), compound (14), compound (15), or a combination thereof, optionally, wherein the un-fixing agent is a composition comprising a compound selected from compound (1), compound (8), or a combination thereof.

In at least one embodiment, the treatment of the composition of fixed cells or nuclei with an un-fixing agent can further comprise incubating the fixed cells or nuclei with a protease; optionally, a cold-active protease. In at least one embodiment, the un-fixing treatment of the dissociated fixed cells or nuclei results from the chop-fix method comprises incubating the cells or nuclei with an un-fixing agent and protease in combination. Methods for the use of a cold-active protease, optionally, together with an un-fixing agent, to prepare biological samples from fixed cells for bulk and partition-based assays of cellular analytes are disclosed in International Patent Application No. PCT/US2021/026592, filed Apr. 9, 2021, which is hereby incorporated by reference herein.

A wide range of proteases are known in the art for use as lysing agents and for releasing cellular analytes from cells or nuclei, tissue samples, and other types of biological samples. Although these proteases are used in methods carried out at room temperature or above, typically at a temperature of 37° C. or higher, in the context of the methods of the present disclosure, it is contemplated that a cold-active (or psychrophilic) protease can be used. Cold-active proteases exhibits at least some measurable proteolytic activity at temperatures as low as 0 C, and typically exhibit significant proteolytic activity in the range of between about 5° C. and about 15° C. As noted above, even proteases that exhibit peak activity at much higher temperatures, such as subtilisin A, can have sufficient low temperature activity to be used as a cold-active protease in the methods of the present disclosure. In at least one embodiment of the present methods, the protease has an average activity at a temperature of between about 5° C. and about 15° C. of at least 1.0 U/mg, at least 5.0 U/mg, at 10.0 U/mg, at least 50 U/mg, at least 100 U/mg, or greater average activity. Determination of average protease activity in the temperature of between about 5° C. and about 15° C. can be carried out by the ordinary artisan using e.g., the well-known universal protease activity assay using casein substrate and Folin-Ciocalteu reagent. Reagents and kits for carrying out such protease activity assays are available commercially (e.g., from Millipore-Sigma; USA).

Accordingly, in at least one embodiment of the method, the protease used in the method is a cold-active protease; optionally, wherein the protease has an average activity of at least 1.0 Units/mg of protease at a temperature of between about 5° C. and about 15° C. In some embodiments, the protease has maximum activity at a temperature of between about 50° C. and about 60° C. Additionally, in some embodiments of the method, it is contemplated that the temperature and time of incubation can be varied somewhat based on the particular protease used and that such conditions can be optimized by one of ordinary skill.

It is also contemplated that the amount of protease used in the treatment can be varied in order to adjust the low temperature proteolytic activity to an effective level. Accordingly, in at least one embodiment of the method, the protease concentration in the solution is between about 1 mg/mL and 100 mg/mL; optionally, the protease concentration in the solution is between about 5 mg/mL and 10 mg/mL.

In at least one embodiment of the method, the protease is a serine protease (E.C. 3.4.21); optionally, wherein the serine protease is selected from chymotrypsin-like, trypsin-like, thrombin-like, elastase-like, and subtilisin-like. A wide range of different serine proteases are well-characterized and commercially available. Among the serine proteases that may be useful in the methods of the present selected are: alcalase, alkaline proteinase, ArcticZymes Proteinase (ArcticZymes Technologies ASA, Tromso, Norway), bacillopeptidase A, bacillopeptidase B, bioprase, colistinase, esperase, genenase, kazusase, maxatase, proteinase K, protease S, savinase, Serratia peptidase (i.e., peptidase derived from Serratia sp.), subtilisin A, subtilisin B, subtilisin BL, subtilisin E, subtilisin J, subtilisin S, subtilisin S41, thermoase, and trypsin.

Proteases have differing substrate preferences and so mixtures of proteases are often used to release cellular analytes or other biological material from cells or nuclei. Accordingly, in some embodiments it is contemplated that the low temperature protease treatment can comprise incubating the fixed biological sample with protease composition. In at least one embodiment, the method of the present disclosure can be carried out wherein the biological sample is incubated with a low-temperature active protease composition comprising at least two different proteases. In some embodiments, the composition comprises at least two proteases selected from: alcalase, alkaline proteinase, ArcticZymes Proteinase, bacillopeptidase A, bacillopeptidase B, bioprase, colistinase, esperase, genenase, kazusase, maxatase, proteinase K, protease S, savinase, Serratia peptidase (i.e., peptidase derived from Serratia sp.), subtilisin A, subtilisin B, subtilisin BL, subtilisin E, subtilisin J, subtilisin S, subtilisin S41, thermoase, and trypsin. For example, in at least one embodiment, a low-temperature active protease composition useful in the methods of the present disclosure comprises subtilisin A and proteinase K.

The un-fixing treatment using an un-fixing agent and low-temperature protease for preparing a sample of un-fixed cells or nuclei from a previously fixed biological sample generally comprises incubating the sample in an aqueous solution containing the protease at a temperature of between about 5° C. and about 15° C. for at least an hour. In another embodiment, the incubating is for between 1 h and 3 h. In some embodiments, the solution further comprises an un-fixing agent that reverses crosslinks between biomolecules of the sample during the low-temperature incubation period. It is also contemplated that in some embodiments, a short period of heating and physical agitation of the sample applied subsequent to the incubation can assist in the sample preparation process without creating artifacts associated with standard high-temperature protease treatments. Accordingly, in at least one embodiment, the method of the present disclosure can be carried out wherein subsequent to incubating the composition of fixed cells or nuclei with an un-fixing agent and protease, the solution is shaken at a temperature of between about 65° C. and 75° C. for at least 15 minutes.

### D. Uses in Bulk and Partition-Based Assay Methods

The methods of the present disclosure that use a chop-fix treatment of tissue samples can be used to prepare samples of fixed or un-fixed dissociated cells from the tissue for use in a range of assay methods. Such assay methods can include “bulk” assays with relatively large sample sizes, or single-cell/single-nucleus assays, such as partition-based (or droplet-based) assays. Exemplary single-cell RNA profiling assays that can be used with chop-fix prepared samples of the present disclosure are described in the Examples, and include the fixed RNA profiling assay described in the patent publications WO2021041974A1, WO2019165318A1, and US20200239874A1, each of which is hereby incorporated by reference herein in its entirety.

The general chop-fix method for preparing a biological sample from tissue as described above and elsewhere herein provides a sample of dissociated fixed cells from the tissue that can be further un-fixed, or used as is in a variety of bulk assays. Accordingly, in at least one embodiment, the present disclosure also provides a method for bulk analysis of a biological tissue, wherein the method comprises: (a) chopping a biological tissue to provide a composition of tissue fragments; (b) treating the composition of tissue fragments with a solution comprising a fixation agent to provide a composition of fixed tissue fragments; (c) treating the composition of fixed tissue fragments with a cell dissociation reagent to provide a composition of fixed cells or nuclei; and (d) further using the composition of fixed cells or nuclei as the input sample for an assay of cellular analytes. As noted above, the composition of fixed cells or nuclei optionally can be treated with an un-fixing agent prior to further use in an assay. It is contemplated that any of the wide range of bulk assays that are well known in the art and that can use dissociated cells as input samples can be used in this context for detecting and quantifying cellular analytes.

The use of dissociated fixed cells or nuclei derived from tissue samples in partition-based assays creates additional challenges due to the small sample amounts and the need to carry out the assay with an extremely small sample volume while maintaining physical separation of the sample. The term “partition,” as used herein, generally, refers to a space or volume that may be suitable to contain one or more species or conduct one or more reactions. A partition may be a physical compartment, such as a droplet or well (e.g., a microwell). The partition may isolate space or volume from another space or volume. The partition may be a droplet of a first phase (e.g., aqueous phase) in a second phase (e.g., oil) that is immiscible with the first phase. The partition may be a droplet of a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

Preparation of a partition containing a biological sample of one or more fixed cells or nuclei that is useful in a partition-based assay involves numerous steps (e.g., sample transport, tissue dissociation, liquid phase washing and transfer, library preparation) that typically take from a few hours to days. During this preparation time an un-fixed biological sample will begin to degrade, and decompose resulting in significant loss of cellular analyte information and thus yield assay results that do not reflect the natural state of the sample.

One type of partition-based assay is a droplet-based assay. Such assays use a biological sample that is isolated and partitioned in discrete droplet in an emulsion. The discrete droplet typically includes a unique identifier for the sample in the form of a unique oligonucleotide sequence also contained in the droplet. The discrete droplet can also contain the assay reagents that are used to generate detectable analytes (e.g., 3′ cDNA sequences) from the sample and provide useful information about it (e.g., RNA transcript profile).

The methods of the present disclosure are useful to prepare a biological sample of a fixed cell or fixed nucleus derived from a tissue encapsulated in discrete droplet along with low-temperature active protease, and an un-fixing agent. The combination of the protease and the un-fixing agent with the fixed sample in the droplet are capable of reversing the fixed state of the biomolecules in the sample while it is sequestered in the droplet. Accordingly, in some embodiments, the present disclosure provides a method for preparing a biological sample comprising: generating a discrete droplet encapsulating a fixed biological sample, a protease composition, and an un-fixing agent. This method can further comprise a step of fixing the biological sample prior to generating the discrete droplet.

In at least one embodiment, the method further comprises generating a discrete droplet encapsulating the biological sample. In at least one embodiment, the method further comprises generating a discrete droplet encapsulating the fixed biological sample and the protease. In at least one embodiment, the method further comprises generating a discrete droplet encapsulating the fixed biological sample, the protease, and the un-fixing agent.

In at least one embodiment wherein the method comprises generating a discrete droplet, the discrete droplet further comprises assay reagents; optionally, wherein the assay reagents are contained in a bead. In at least one embodiment, the discrete droplet further comprises a barcode; optionally, wherein the barcode contained in a bead.

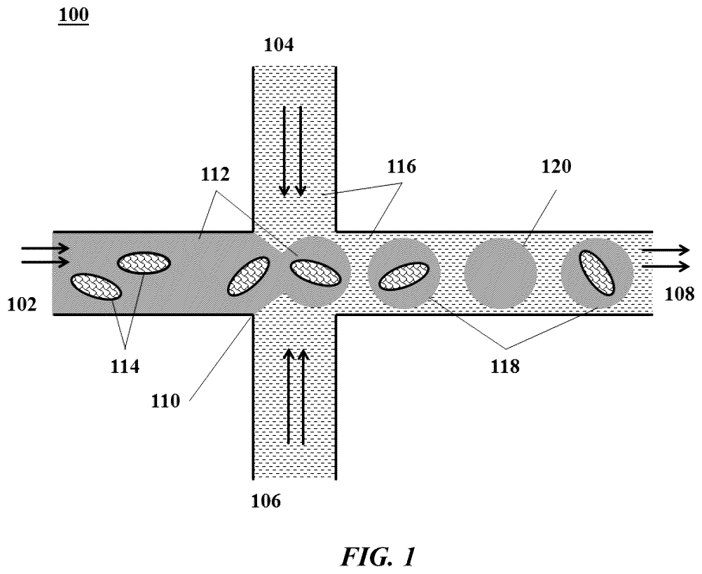

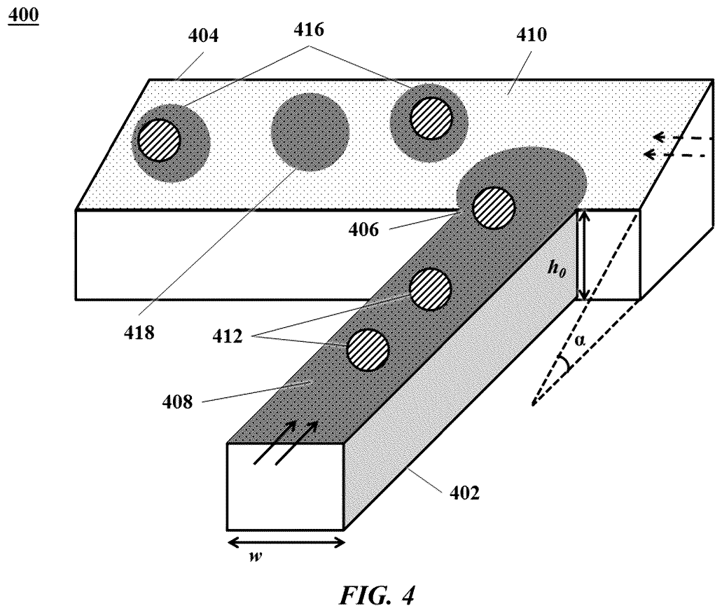

Methods, techniques, and protocols useful for partitioning biological samples (e.g., individual cells, individual cell nuclei, biomolecular contents of cells, etc.) into discrete droplets are known and well described in the art. The discrete droplets generated act a nanoliter-scale container that can maintain separation the droplet contents from the contents of other droplets in the emulsion. Methods and systems for creating stable discrete droplets encapsulating individual particles from biological samples in non-aqueous or oil emulsions are described in, e.g., U.S. Patent Application Publication Nos. 2010/0105112 and 2019/0100632, each of which is entirely incorporated herein by reference for all purposes. Briefly, discrete droplets in an emulsion encapsulating a biological sample is accomplished by introducing a flowing stream of an aqueous fluid containing the biological sample into a flowing stream of a non-aqueous fluid with which it is immiscible, such that droplets are generated at the junction of the two streams (see FIGS. 1-3). By providing the aqueous stream at a certain concentration and/or flow rate of the biological sample, the occupancy of the resulting droplets can be controlled. For example, the relative flow rates of the immiscible fluids can be selected such that, on average, the discrete droplet each contains less than one biological particle. Such a flow rate ensures that the droplets that are occupied are primarily occupied by a single sample (e.g., a single cell or a single cell nucleus). Discrete droplets in an emulsion encapsulating a biological sample is also accomplished using a microfluidic architecture comprising a channel segment having a channel junction with a reservoir (see FIGS. 4-6).

The term “biological particle,” as used herein, generally refers to a discrete biological system derived from a biological sample. The biological particle may be a macromolecule. The biological particle may be a small molecule. The biological particle may be a virus. The biological particle may be a cell or derivative of a cell. The biological particle may be an organelle from a cell. Examples of an organelle from a cell include, without limitation, a nucleus, endoplasmic reticulum, a ribosome, a Golgi apparatus, an endoplasmic reticulum, a chloroplast, an endocytic vesicle, an exocytic vesicle, a vacuole, and a lysosome. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle.

In some cases, the droplets among a plurality of discrete droplets formed in the manner contain at most one particle (e.g., one bead, one cell or one nucleus). The flows and microfluidic channel architectures also can be controlled to ensure a given number of singly occupied droplets, less than a certain level of unoccupied droplets, and/or less than a certain level of multiply occupied droplets.

In another aspect of the disclosure, fixed cells or nuclei, protease composition, and optional un-fixing agent composition may then be partitioned (e.g., in a droplet or well) with other reagents for processing of one or more analytes as described herein. In one embodiment, the fixed cell or cell nucleus, protease composition, and optional un-fixing agent composition may be partitioned with a support (e.g., a bead) comprising nucleic acid molecules suitable for barcoding of the one or more analytes. In another embodiment, the nucleic acid molecules may include nucleic acid sequences that provide identifying information, e.g., barcode sequence(s).

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence (e.g., targeted by a primer sequence) or a non-targeted sequence. For example, in the methods, compositions, kits, and systems described herein, hybridization and reverse transcription of the nucleic acid molecule (e.g., a messenger RNA (mRNA) molecule) of a cell or nucleus with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the mRNA.

The term “bead,” as used herein, generally refers to a particle. The bead may be a solid or semi-solid particle. The bead may be a gel bead. The gel bead may include a polymer matrix (e.g., matrix formed by polymerization or crosslinking). The polymer matrix may include one or more polymers (e.g., polymers having different functional groups or repeat units). Polymers in the polymer matrix may be randomly arranged, such as in random copolymers, and/or have ordered structures, such as in block copolymers. Crosslinking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold, or silver) covered with a coating comprising one or more polymers. Such coating may be disruptable or dissolvable.

FIG. 1 shows an exemplary microfluidic channel structure 100 useful for generating discrete droplets encapsulating a particle from a biological sample, such as a single cell or single nucleus. The channel structure 100 can include channel segments 102, 104, 106 and 108 communicating at a channel junction 110. In operation, a first aqueous fluid 112 that that includes suspended particles (e.g., cells or nuclei) from a biological sample 114 are transported along channel segment 102 into junction 110, while a second fluid 116 (or “partitioning fluid”) that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual particle from a biological sample 114 (such as droplet 118), or discrete droplet can be generated that includes more than one particle 114 (not shown in FIG. 1). A discrete droplet may contain no biological particle 114 (such as droplet 120). Each discrete droplet is capable of maintaining separation of its own contents (e.g., individual biological particle 114) from the contents of other droplets.

Typically, the second fluid 116 comprises an oil, such as a fluorinated oil, that includes a fluoro-surfactant that helps to stabilize the resulting droplets. Examples of useful partitioning fluids and fluoro-surfactants are described in e.g., U.S. Patent Application Publication No. 2010/0105112, which is entirely incorporated herein by reference for all purposes.

The microfluidic channels for generating discrete droplets as exemplified in FIG. 1 may be coupled to any of a variety of different fluid sources or receiving components, including reservoirs, tubing, manifolds, or fluidic components of other systems. Additionally, the microfluidic channel structure 100 may have other geometries, including geometries having more than one channel junction. For example, the microfluidic channel structure can have 2, 3, 4, or 5 channel segments each carrying biological particles from a biological sample, assay reagents, and/or beads that meet at a channel junction.

Generally, the fluids used in generating the discrete droplets are directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electro-kinetic pumping, vacuum, capillary or gravity flow, or the like.

One of ordinary skill will recognize that numerous different microfluidic channel designs are available that can be used with the methods of the present disclosure to provide discrete droplets containing a biological particle from a fixed biological sample, a protease composition, an un-fixing agent composition, and/or a bead with a barcode and/or other assay reagents.

The inclusion of a barcode in a discrete droplet along with the biological sample provides a unique identifier that allows data from the biological sample to be distinguished and individually analyzed. Barcodes can be delivered previous to, subsequent to, or concurrent with the biological sample in discrete droplet. For example, barcodes may be injected into droplets previous to, subsequent to, or concurrently with droplet generation. Barcodes useful in the methods of the present disclosure typically comprise a nucleic acid molecule (e.g., an oligonucleotide). The nucleic acid barcode molecules typically are delivered to a partition via a support, such as a bead. In some cases, barcode nucleic acid molecules are initially associated with the bead upon generation of the discrete droplet, and then released from the bead upon application of a stimulus to droplet. Barcode carrying beads useful in the methods of the present disclosure are described in further detail elsewhere herein.

Methods and systems for partitioning barcode carrying beads into droplets are provided in U.S. Pat. Nos. 10,480,029, 10,858,702, and 10,725,027, US. Patent Publication Nos. 2019/0367997 and 2019/0064173, and International Application Nos. PCT/US20/17785 and PCT/US20/020486, each of which is herein entirely incorporated by reference for all purposes.

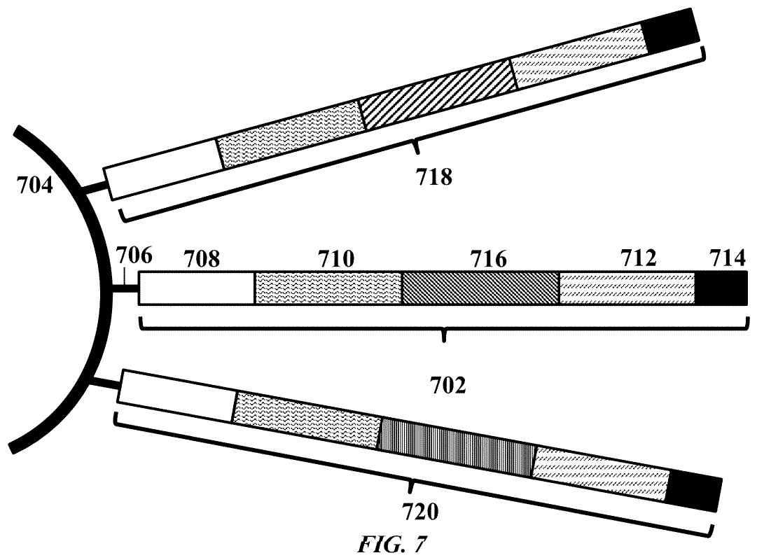

FIG. 7 illustrates an example of a barcode carrying bead. A nucleic acid molecule 702, such as an oligonucleotide, can be coupled to a bead 704 by a releasable linkage 706, such as, for example, a disulfide linker. The same bead 704 may be coupled (e.g., via releasable linkage) to one or more other nucleic acid molecules 718, 720. The nucleic acid molecule 702 may be or comprise a barcode. As noted elsewhere herein, the structure of the barcode may comprise a number of sequence elements. The nucleic acid molecule 702 may comprise a functional sequence 708 that may be used in subsequent processing. For example, the functional sequence 708 may include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 702 may comprise a barcode sequence 710 for use in barcoding the sample (e.g., DNA, RNA, protein, antibody, etc.). In some cases, the barcode sequence 710 can be bead-specific such that the barcode sequence 710 is common to all nucleic acid molecules (e.g., including nucleic acid molecule 702) coupled to the same bead 704. Alternatively, or in addition, the barcode sequence 710 can be partition-specific such that the barcode sequence 710 is common to all nucleic acid molecules coupled to one or more beads that are partitioned into the same partition. The nucleic acid molecule 702 may comprise a specific priming sequence 712, such as an mRNA specific priming sequence (e.g., poly-T sequence), a targeted priming sequence, and/or a random priming sequence. The nucleic acid molecule 702 may comprise an anchoring sequence 714 to ensure that the specific priming sequence 712 hybridizes at the sequence end (e.g., of the mRNA). For example, the anchoring sequence 714 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 702 may comprise a unique molecular identifying sequence 716 (e.g., unique molecular identifier (UMI)). In some cases, the unique molecular identifying sequence 716 may comprise from about 5 to about 8 nucleotides. Alternatively, the unique molecular identifying sequence 716 may compress less than about 5 or more than about 8 nucleotides. The unique molecular identifying sequence 716 may be a unique sequence that varies across individual nucleic acid molecules (e.g., 702, 718, 720, etc.) coupled to a single bead (e.g., bead 704). In some cases, the unique molecular identifying sequence 716 may be a random sequence (e.g., such as a random N-mer sequence). For example, the UMI may provide a unique identifier of the starting mRNA molecule that was captured, in order to allow quantitation of the number of original expressed RNA. As will be appreciated, although FIG. 7 shows three nucleic acid molecules 702, 718, 720 coupled to the surface of the bead 704, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 708, 710, 712, etc.) and variable or unique sequence segments (e.g., 716) between different individual nucleic acid molecules coupled to the same bead.

A biological particle (e.g., cell or nucleus, fixed cell or nucleus, un-fixed cell or nucleus, DNA, RNA, etc.) can be co-partitioned along with a barcode bearing bead 704. The barcoded nucleic acid molecules 702, 718, 720 can be released from the bead 704 in the partition. By way of example, in the context of analyzing sample RNA, the poly-T segment (e.g., 712) of one of the released nucleic acid molecules (e.g., 702) can hybridize to the poly-A tail of a mRNA molecule. Reverse transcription may result in a cDNA transcript of the mRNA, but which transcript includes each of the sequence segments 708, 710, 716 of the nucleic acid molecule 702. Because the nucleic acid molecule 702 comprises an anchoring sequence 714, it will more likely hybridize to and prime reverse transcription at the sequence end of the poly-A tail of the mRNA. Within any given partition, all of the cDNA transcripts of the individual mRNA molecules may include a common barcode sequence segment 710.

However, the transcripts made from the different mRNA molecules within a given partition may vary at the unique molecular identifying sequence 712 segment (e.g., UMI segment). Beneficially, even following any subsequent amplification of the contents of a given partition, the number of different UMIs can be indicative of the quantity of mRNA originating from a given partition, and thus from the biological particle (e.g., a cell or nucleus, a fixed cell or nucleus, an un-fixed cell or nucleus, etc.). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing). The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where the nucleic acid molecules from the sample remain attached to the bead, the beads from various partitions may be collected, pooled, and subjected to further processing (e.g., reverse transcription, adapter attachment, amplification, clean up, sequencing). In other instances, the processing may occur in the partition. For example, conditions sufficient for barcoding, adapter attachment, reverse transcription, or other nucleic acid processing operations may be provided in the partition and performed prior to clean up and sequencing.

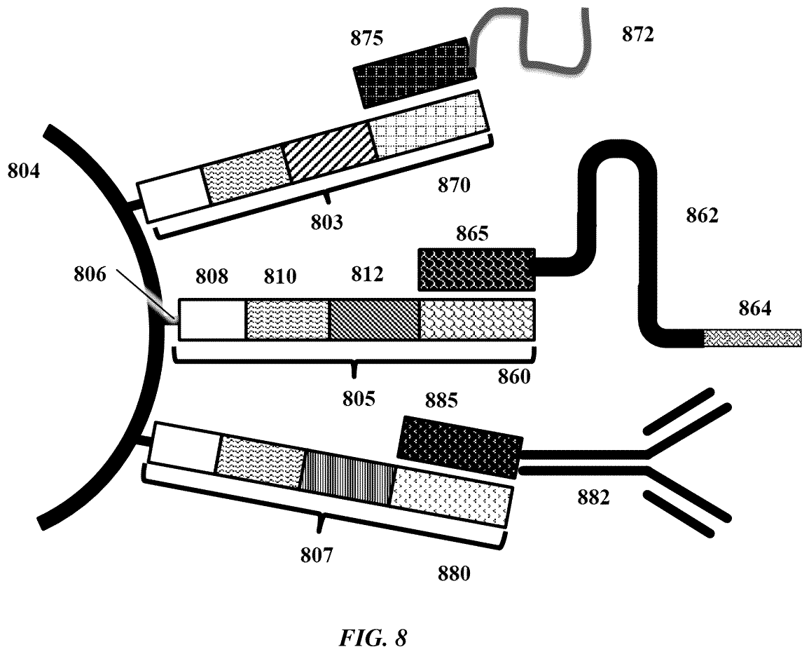

FIG. 8 illustrates another example of a barcode carrying bead. A nucleic acid molecule 805, such as an oligonucleotide, can be coupled to a bead 804 by a releasable linkage 806, such as, for example, a disulfide linker. The nucleic acid molecule 805 may comprise a first capture sequence 860. The same bead 804 may be coupled (e.g., via releasable linkage) to one or more other nucleic acid molecules 803, 807 comprising other capture sequences. The nucleic acid molecule 805 may be or comprise a barcode. As noted elsewhere herein, the structure of the barcode may comprise a number of sequence elements, such as a functional sequence 808 (e.g., flow cell attachment sequence, sequencing primer sequence, etc.), a barcode sequence 810 (e.g., bead-specific sequence common to bead, partition-specific sequence common to partition, etc.), and a unique molecular identifier 812 (e.g., unique sequence within different molecules attached to the bead), or partial sequences thereof. The capture sequence 860 may be configured to attach to a corresponding capture sequence 865. In some instances, the corresponding capture sequence 865 may be coupled to another molecule that may be an analyte or an intermediary carrier. For example, as illustrated in FIG. 8, the corresponding capture sequence 865 is coupled to a guide RNA molecule 862 comprising a target sequence 864, wherein the target sequence 864 is configured to attach to the analyte. Another oligonucleotide molecule 807 attached to the bead 804 comprises a second capture sequence 880 which is configured to attach to a second corresponding capture sequence 885. As illustrated in FIG. 8, the second corresponding capture sequence 885 is coupled to an antibody 882. In some cases, the antibody 882 may have binding specificity to an analyte (e.g., surface protein). Alternatively, the antibody 882 may not have binding specificity. Another oligonucleotide molecule 803 attached to the bead 804 comprises a third capture sequence 870 which is configured to attach to a second corresponding capture sequence 875. As illustrated in FIG. 8, the third corresponding capture sequence 875 is coupled to a molecule 872. The molecule 872 may or may not be configured to target an analyte. The other oligonucleotide molecules 803, 807 may comprise the other sequences (e.g., functional sequence, barcode sequence, UMI, etc.) described with respect to oligonucleotide molecule 805. While a single oligonucleotide molecule comprising each capture sequence is illustrated in FIG. 8, it will be appreciated that, for each capture sequence, the bead may comprise a set of one or more oligonucleotide molecules each comprising the capture sequence. For example, the bead may comprise any number of sets of one or more different capture sequences. Alternatively, or in addition, the bead 804 may comprise other capture sequences. Alternatively, or in addition, the bead 804 may comprise fewer types of capture sequences (e.g., two capture sequences). Alternatively or in addition, the bead 804 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

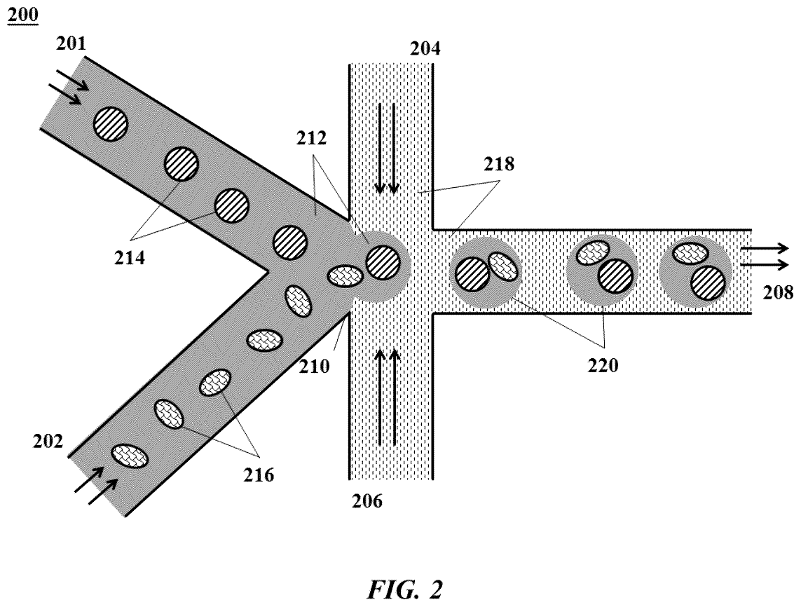

FIG. 2 shows an exemplary microfluidic channel structure 200 for generating discrete droplets encapsulating a barcode carrying bead 214 along with a biological particle 216. The channel structure 200 includes channel segments 201, 202, 204, 206 and 208 in fluid communication at a channel junction 210. In operation, the channel segment 201 transports an aqueous fluid 212 that can include a plurality of beads 214 (e.g., gel beads carrying barcode oligonucleotides) along the channel segment 201 into junction 210. The plurality of beads 214 may be sourced from a suspension of beads. For example, the channel segment 201 can be connected to a reservoir comprising an aqueous suspension of beads 214. The channel segment 202 transports the aqueous fluid 212 that includes a plurality of biological particles from a biological sample 216 along the channel segment 202 into junction 210. The plurality of biological particles 216 may be sourced from a suspension of biological sample. For example, the channel segment 202 may be connected to a reservoir comprising an aqueous suspension of biological particles 216. In some instances, the aqueous fluid 212 in either the first channel segment 201 or the second channel segment 202, or in both segments, can include one or more reagents, as further described elsewhere herein. For example, in some embodiments of the present disclosure, where the biological particles are from a fixed biological sample, the aqueous fluid in the first and/or second channel segments that delivers the biological sample and beads, respectively, can include an un-fixing agent. The second fluid 218 that is immiscible with the aqueous fluid 212 is delivered to the junction 210 from each of channel segments 204 and 206. Upon meeting of the aqueous fluid 212 from each of channel segments 201 and 202 and the second fluid 218 (e.g., a fluorinated oil) from each of channel segments 204 and 206 at the channel junction 210, the aqueous fluid 212 is partitioned into discrete droplets 220 in the second fluid 218 and flow away from the junction 210 along channel segment 208. The channel segment 208 can then deliver the discrete droplets encapsulating the biological particle and barcode carrying bead to an outlet reservoir fluidly coupled to the channel segment 208, where they can be collected.

As an alternative, the channel segments 201 and 202 may meet at another junction upstream of the junction 210. At such junction, beads and biological particles may form a mixture that is directed along another channel to the junction 210 to yield droplets 220. The mixture may provide the beads and biological particles in an alternating fashion, such that, for example, a droplet comprises a single bead and a single biological particle.

Using such a channel system as exemplified in FIG. 2, discrete droplets 220 can be generated that encapsulate an individual particle of a biological sample, and one bead, wherein the bead can carry a barcode and/or another reagent. It is also contemplated, that in some instances, a discrete droplet may be generated using the channel system of FIG. 2, wherein droplet includes more than one individual biological particle or includes no biological sample. Similarly, in some embodiments, the discrete droplet may include more than one bead or no bead. A discrete droplet also may be completely unoccupied (e.g., no bead or biological sample).

In some embodiments, it is desired that the beads, biological particles from a biological sample, and generated discrete droplets flow along channels at substantially regular flow rates that generate a discrete droplet containing a single bead and a single biological particle. Regular flow rates and devices that may be used to provide such regular flow rates are known in the art, see e.g., U.S. Patent Publication No. 2015/0292988, which is hereby incorporated by reference herein in its entirety. In some embodiments, the flow rates are set to provide discrete droplets containing a single bead and a biological particle with a yield rate of greater than 5%, 10%, 20%, 30%, 40%, 50%, 60%, 70%, 80%, 90%, or 95%.

### E. Supports

Supports, such as beads, that can carry barcodes and/or other reagents are useful with the methods of the present disclosure and can include, without limitation, supports that are porous, non-porous, solid, semi-solid, semi-fluidic, fluidic, and/or a combination thereof. In some embodiments, the support is a bead that is made of a material that is dissolvable, disruptable, and/or degradable, such as a gel bead comprising a hydrogel. Alternatively, in some embodiments, the support is not degradable.

In some embodiments of the present disclosure, the support is a bead that can be encapsulated in a discrete droplet with a biological sample. Typically, the bead useful in the embodiments disclosed herein comprise a hydrogel. Such gel beads can be formed from molecular precursors, such as a polymeric or monomeric species, that undergo a reaction to form crosslinked gel polymer. Another semi-solid bead useful in the present disclosure is a liposomal bead. In some embodiments, beads used can be solid beads that comprise a metal including iron oxide, gold, and silver. In some cases, the bead may be a silica bead. In some cases, the bead can be rigid. In other cases, the bead may be flexible and/or compressible. Generally, the beads can be of any suitable shape. Examples of bead shapes include, but are not limited to, spherical, non-spherical, oval, oblong, amorphous, circular, cylindrical, and variations thereof.

In some embodiments, a plurality or population of beads can be used. The plurality of beads used in the embodiments can be of uniform size, having a relatively monodisperse size distribution, or they can comprise a collection of heterogeneous sizes. In some cases, the diameter of a bead is at least about 1 micron (μm), 5 μm, 10 μm, 20 μm, 30 μm, 40 μm, 50 μm, 60 μm, 70 μm, 80 μm, 90 μm, 100 μm, 250 μm, 500 μm, 1000 μm (1 mm), or greater. In some cases, a bead may have a diameter of less than about 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

Typically, where it is desirable to provide a consistent amount of a reagent within a discrete droplet, the use of relatively consistent bead characteristics, such as size, provides overall consistency in the content of each droplet. For example, the beads useful in the embodiments of the present disclosure can have size distributions that have a coefficient of variation in their cross-sectional dimensions of less than 50%, less than 40%, less than 30%, less than 20%, and in some cases less than 15%, less than 10%, less than 5%, or less.

The beads useful in the methods of the present disclosure can comprise a range of natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

Although FIG. 1 and FIG. 2 have been described in terms of providing substantially singly occupied discrete droplets, it is also contemplated in certain embodiments that it is desirable to provide multiply occupied discrete droplets, e.g., a single droplet that contains two, three, four or more cells/nuclei from a biological sample, and/or multiple different beads, such as a bead carrying a barcode nucleic acid molecule and/or a support (e.g., a bead) carrying a reagent such as an un-fixing agent or assay reagent. Accordingly, as noted elsewhere herein, the flow characteristics of the biological particle and/or the beads can be controlled to provide for such multiply occupied droplets. In particular, the flow parameters of the liquids used in the channel structures may be controlled to provide a given droplet occupancy rate greater than about 50%, greater than about 75%, and in some cases greater than about 80%, 90%, 95%, or higher.

In some embodiments, the beads useful in the methods of the present disclosure are supports (e.g., beads) capable of delivering reagents (e.g., an un-fixing agent, and/or an assay reagent) into the discrete droplet generated containing the biological particle. In some embodiments, the different beads (e.g., containing different reagents) can be introduced from different sources into different inlets leading to a common droplet generation junction (e.g., junction 210). In such cases, the flow and frequency of the different beads into the channel or junction may be controlled to provide for a certain ratio of supports from each source, while ensuring a given pairing or combination of such supports (e.g., beads) into a partition with a given number of biological particles (e.g., one biological particle and one bead per partition).

The discrete droplets described herein generally comprise small volumes, for example, less than about 10 microliters (L), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less. In some embodiments, the discrete droplets generated that encapsulate a biological particle have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. It will be appreciated that the sample fluid volume, e.g., including co-partitioned biological particles and/or beads, within the droplets may be less than about 90% of the above described volumes, less than about 80%, less than about 70%, less than about 60%, less than about 50%, less than about 40%, less than about 30%, less than about 20%, or less than about 10% of the above described volumes.

The methods of generating discrete droplets useful with the methods of the present disclosure, result in the generation of a population or plurality of discrete droplets containing a biological particle (e.g., a biological particle from a fixed biological sample) and other reagents (e.g., an un-fixing agent). Generally, the methods are easily controlled to provide for any suitable number of droplets. For example, at least about 1,000 discrete droplets, at least about 5,000 discrete droplets, at least about 10,000 discrete droplets, at least about 50,000 discrete droplets, at least about 100,000 discrete droplets, at least about 500,000 discrete droplets, at least about 1,000,000 discrete droplets, at least about 5,000,000 discrete droplets, at least about 10,000,000 discrete droplets, or more discrete droplets can be generated or otherwise provided. Moreover, the plurality of discrete droplets may comprise both unoccupied and occupied droplets.

As described elsewhere herein, in some embodiments of the methods of the present disclosure, the generated discrete droplets encapsulating a biological particle, and optionally, one or more different beads, also contain other reagents. In some embodiments, the other reagents encapsulated in the droplet include lysis and/or un-fixing agents that act to release and/or un-fix the biomolecule contents of the biological particle within the droplet. In some embodiments, the lysis and/or un-fixing agents can be contacted with the biological sample suspension concurrently with, or immediately prior to, the introduction of the biological particles into the droplet generation junction of the microfluidic system (e.g., junction 210). In some embodiments, the agents are introduced through an additional channel or channels upstream of the channel junction.

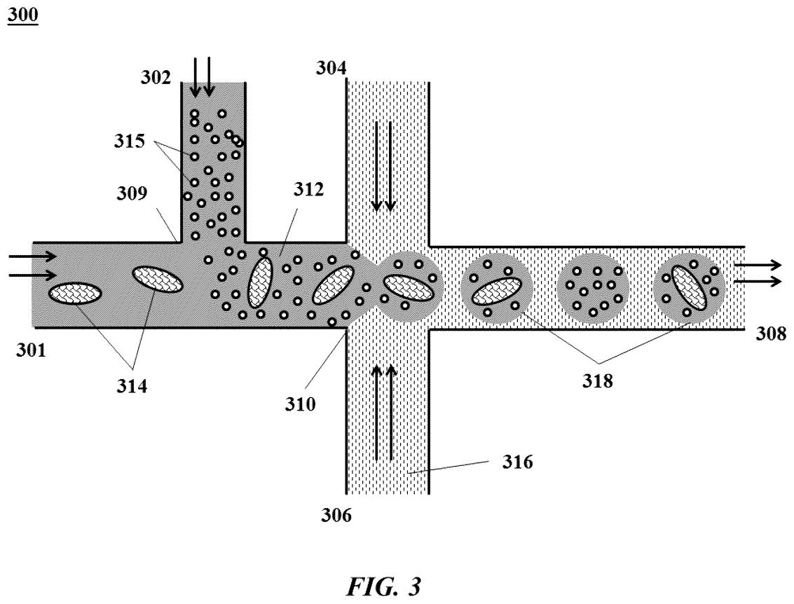

In some embodiments, a biological particle can be co-partitioned along with the other reagents. FIG. 3 shows an example of a microfluidic channel structure 300 for co-partitioning biological particles and other reagents, including lysis and/or un-fixing agents. The channel structure 300 can include channel segments 301, 302, 304, 306 and 308. Channel segments 301 and 302 communicate at a first channel junction 309. Channel segments 302, 304, 306, and 308 communicate at a second channel junction 310. In exemplary co-partitioning operation, the channel segment 301 may transport an aqueous fluid 312 that includes a plurality of biological particles 314 (e.g., a fixed biological sample) along the channel segment 301 into the second junction 310. As an alternative or in addition to, channel segment 301 may transport beads (e.g., beads that carry barcodes). For example, the channel segment 301 may be connected to a reservoir comprising an aqueous suspension of biological particles 314. Upstream of, and immediately prior to reaching, the second junction 310, the channel segment 301 may meet the channel segment 302 at the first junction 309. The channel segment 302 can transport a plurality of reagents 315 (e.g., lysis or un-fixing agents) in the aqueous fluid 312 along the channel segment 302 into the first junction 309. For example, the channel segment 302 may be connected to a reservoir comprising the reagents 315. After the first junction 309, the aqueous fluid 312 in the channel segment 301 can carry both the biological particles 314 and the reagents 315 towards the second junction 310. In some instances, the aqueous fluid 312 in the channel segment 301 can include one or more reagents, which can be the same or different reagents as the reagents 315. A second fluid 316 that is immiscible with the aqueous fluid 312 (e.g., a fluorinated oil) can be delivered to the second junction 310 from each of channel segments 304 and 306. Upon meeting of the aqueous fluid 312 from the channel segment 301 and the second fluid 316 from each of channel segments 304 and 306 at the second channel junction 310, the aqueous fluid 312 is partitioned as discrete droplets 318 in the second fluid 316 and flow away from the second junction 310 along channel segment 308. The channel segment 308 may deliver the discrete droplets 318 to an outlet reservoir fluidly coupled to the channel segment 308, where they may be collected for further analysis.

Discrete droplets generated can include an individual biological particle 314 and/or one or more reagents 315, depending on what reagents are included in channel segment 302. In some instances, a discrete droplet generated may also include a barcode carrying bead (not shown), such as can be added via other channel structures described elsewhere herein. In some instances, a discrete droplet may be unoccupied (e.g., no reagents, no biological particles). Generally, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electro-kinetic pumping, vacuum, capillary or gravity flow, or the like.

FIG. 4 shows an example of a microfluidic channel structure for the controlled partitioning of beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 may be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the junction 406 where the aqueous fluid 408 and the second fluid 410 meet, droplets can form based on factors such as the hydrodynamic forces at the junction 406, flow rates of the two fluids 408, 410, fluid properties, and certain geometric parameters (e.g., w, h₀, α, etc.) of the channel structure 400. A plurality of droplets can be collected in the reservoir 404 by continuously injecting the aqueous fluid 408 from the channel segment 402 through the junction 406.

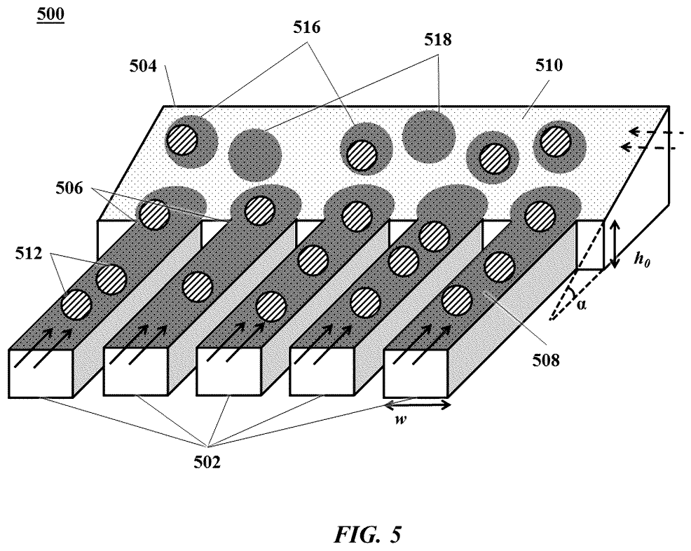

FIG. 5 shows an example of a microfluidic channel structure for increased droplet generation throughput. A microfluidic channel structure 500 can comprise a plurality of channel segments 502 and a reservoir 504. Each of the plurality of channel segments 502 may be in fluid communication with the reservoir 504. The channel structure 500 can comprise a plurality of channel junctions 506 between the plurality of channel segments 502 and the reservoir 504. Each channel junction can be a point of droplet generation. The channel segment 402 from the channel structure 400 in FIG. 4 and any description to the components thereof may correspond to a given channel segment of the plurality of channel segments 502 in channel structure 500 and any description to the corresponding components thereof. The reservoir 404 from the channel structure 400 and any description to the components thereof may correspond to the reservoir 504 from the channel structure 500 and any description to the corresponding components thereof.

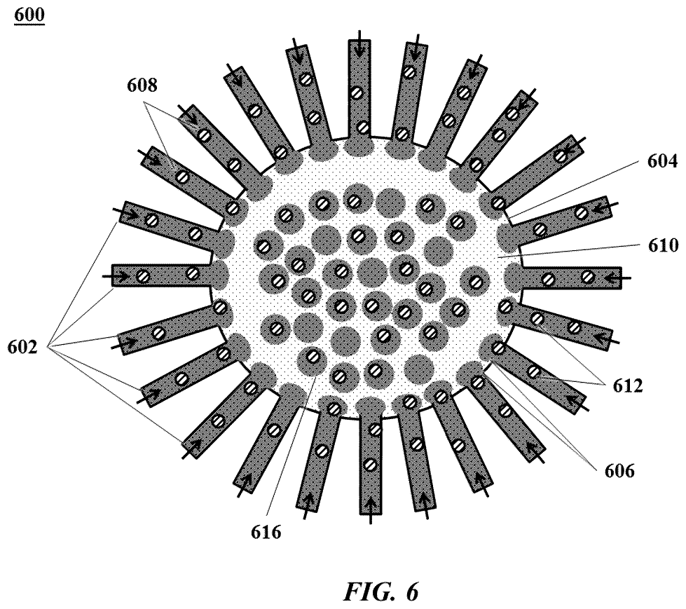

FIG. 6 shows another example of a microfluidic channel structure for increased droplet generation throughput. A microfluidic channel structure 600 can comprise a plurality of channel segments 602 arranged generally circularly around the perimeter of a reservoir 604. Each of the plurality of channel segments 602 may be in fluid communication with the reservoir 604. The channel structure 600 can comprise a plurality of channel junctions 606 between the plurality of channel segments 602 and the reservoir 604. Each channel junction can be a point of droplet generation. The channel segment 402 from the channel structure 400 in FIG. 4 and any description to the components thereof may correspond to a given channel segment of the plurality of channel segments 602 in channel structure 600 and any description to the corresponding components thereof. The reservoir 404 from the channel structure 400 and any description to the components thereof may correspond to the reservoir 604 from the channel structure 600 and any description to the corresponding components thereof. Additional aspects of the microfluidic structures depicted in FIGS. 4-6, including systems and methods implementing the same, are provided in US Published Patent Application No 20190323088, which is incorporated herein by reference in its entirety.

Once the lysis and/or un-fixing agents are co-partitioned in a droplet with a fixed biological particle, these reagents can facilitate the release and un-fixing of the biomolecular contents of the biological particle within the droplet. As described elsewhere herein, the un-fixed biomolecular contents released in a droplet remain discrete from the contents of other droplets, thereby allowing for detection and quantitation of the biomolecular analytes of interest present in that distinct biological sample.

Examples of lysis agents useful in the methods of the present disclosure include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological samples' contents into the droplet. For example, in some cases, surfactant-based lysis solutions may be used to lyse cells or nuclei, although these may be less desirable for emulsion based systems where the surfactants can interfere with stable emulsions. In some embodiment, the lysis solutions can include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

In addition to the lysis and/or un-fixing agents co-partitioned into discrete droplets with the biological particles, it is further contemplated that other assay reagents can also be co-partitioned in the droplet. For example, DNase and RNase inactivating agents or inhibitors, such as proteinase K, chelating agents, such as EDTA, proteases, such as subtilisin A, and other reagents employed in removing or otherwise reducing negative activity or impact of different cell lysate components on subsequent processing of nucleic acids.

In some embodiments, the biological particles from a biological sample are provided in or encapsulated in discrete partitions (e.g., wells or droplets) with other reagents are exposed to an appropriate stimulus to release the biomolecular contents of the sample particles and/or the contents of a co-partitioned support (e.g., a bead). For example, in some embodiments, a chemical stimulus may be co-partitioned in the droplet along with a biological particle and a support (e.g., a bead such as a gel bead) to allow for the degradation of the support and release of the its contents into the droplet. In some embodiments, a discrete droplet can be generated with a fixed biological particle and an un-fixing agent, wherein the un-fixing agent is contained in a support (e.g., a bead) that can be degraded by heat stimulus. In such an embodiment, the droplet is exposed to heat stimulus thereby degrading the bead and releasing the un-fixing agent. In another embodiment, it is contemplated that a droplet encapsulating a fixed biological particle from a fixed biological sample, and two different beads (e.g., one bead carrying an un-fixing agent, and one bead carrying assay reagents), wherein the contents of the two different beads are released by non-overlapping stimuli (e.g., a chemical stimulus and a heat stimulus). Such an embodiment can allow the release of the different reagents into the same discrete droplet at different times. For example, a first bead, triggered by heat stimulus, releases an un-fixing agent into the droplet, and then after a set time, a second bead, triggered by a chemical stimulus, releases assay reagents that detect analytes of the un-fixed biological particle.

Additional assay reagents may also be co-partitioned into discrete droplets with the biological samples, such as endonucleases to fragment a biological sample's DNA, DNA polymerase enzymes and dNTPs used to amplify the biological sample's nucleic acid fragments and to attach the barcode molecular tags to the amplified fragments. Other enzymes may be co-partitioned, including without limitation, polymerase, transposase, ligase, proteinase K, DNase, subtilisin A, etc. Additional assay reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching.

In some embodiments, template switching can be used to increase the length of cDNA generated in an assay. In some embodiments, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner.

Once the contents of a biological sample cell or nucleus are released into a discrete droplet, the biomolecular components (e.g., macromolecular constituents of biological samples, such as RNA, DNA, or proteins) contained therein may be further processed within the droplet. In accordance with the methods and systems described herein, the biomolecular contents of individual biological samples can be provided with unique barcode identifiers, and upon characterization of the biomolecular components (e.g., in a sequencing assay) they may be attributed as having been derived from the same biological sample. The ability to attribute characteristics to individual biological samples or groups of biological samples is provided by the assignment of a nucleic acid barcode sequence specifically to an individual biological sample or groups of biological samples.

In some aspects, the unique identifier barcodes are provided in the form of nucleic acid molecules (e.g., oligonucleotides) that comprise sequences that may be attached to or otherwise associated with the nucleic acid contents of individual biological sample, or to other components of the biological sample, and particularly to fragments of those nucleic acids. In some embodiments, only one nucleic acid barcode sequence is associated with a given discrete droplet, although in some cases, two or more different barcode sequences may be present. The nucleic acid barcode sequences can include from about 6 to about 20 or more nucleotides within the sequence of the nucleic acid molecules (e.g., oligonucleotides). In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

In some embodiments, the nucleic acid barcode molecules can also comprise other functional sequences useful in the processing of the nucleic acids from the biological sample in the droplet. These functional sequences can include, e.g., targeted or random/universal amplification primer sequences for amplifying the nucleic acid molecules from the individual biological samples within the partitions while attaching the associated barcode sequences, sequencing primers or primer recognition sites, hybridization or probing sequences, e.g., for identification of presence of the sequences or for pulling down barcoded nucleic acid molecules, or any of a number of other potential functional sequences.

In some embodiments, large numbers of nucleic acid barcode molecules (e.g., oligonucleotides) are releasably attached to beads, wherein all of the nucleic acid molecules attached to a particular bead will include the same nucleic acid barcode sequence, but where a large number of diverse barcode sequences are represented across the population of beads used. In some embodiments, gel beads (e.g., comprising polyacrylamide polymer matrices), are used as a solid support and delivery vehicle for the nucleic acid molecules into the droplets, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more.

The nucleic acid barcode molecules can be released from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules form the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological samples and may be degraded for release of the attached nucleic acid molecules through exposure to a reducing agent, such as DTT.

F. Use of Fixed and Un-Fixed Cells from Tissue in Partition-Based Assays

As disclosed elsewhere herein, the chop-fix methods of the present disclosure proved a sample of dissociate fixed cells from biological tissue (e.g., formaldehyde-fixed biopsy cells). These fixed cells can then be provided in a discrete partition (e.g., encapsulated in a droplet), optionally, as a single cell or nucleus, optionally, together with an un-fixing agent and/or a low-temperature active protease, capable of reversing the fixation. The optional un-fixing agent and/or protease treatment can act to release and un-fix the cellular analytes within the sample (e.g., cell/nucleus, cells/nuclei, tissue sample, or other type of biological sample). In some assay, it may be desirable to carry out the un-fixing treatment to provide cellular analytes for assay that more closely resemble analytes from a fresh sample.

Significantly, the chop-fix methods of the present disclosure allow for a fresh tissue sample to be collected, immediately chopped and fixed (e.g., with formaldehyde), and then stored for a period of time before it is subjected to dissociation into fixed cells, or optionally further subjected to an un-fixing treatment, prior to use in a partition-based assay. Accordingly, it is contemplated that the methods of the present disclosure can be carried out wherein the amount of time prior to generating a partition containing a sample cell from the tissue is at least 1 hour, at least 2 hours, at least 6 hours, at least 12 hours, at least 24 hours, at least 1 week, at least 1 month, at least 6 months, or longer.

In some aspects, the tissue sample is exposed to room temperature or greater for at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 3 days, 4 days, 5 days after collecting the tissue sample from a eukaryotic organism; and immediately thereafter fixing the tissue sample. In some aspects, the tissue sample is exposed to a temperature of at least about 15° C., 20° C., 25° C., 30° C., 35° C., 40° C., 50° C., 60° C., 70° C., 80° C., or 90° C. for a duration described herein prior to fixation. In some aspects, the tissue sample is exposed to a temperature between 15° C. and 90° C., 15° C. and 80° C., 15° C. and 70° C., 15° C. and 60° C., 15° C. and 50° C., 15° C. and 40° C., 15° C. and 35° C., 15° C. and 30° C., 15° C. and 25° C., 25° C. and 90° C., 25° C. and 80° C., 25° C. and 70° C., 25° C. and 60° C., 25° C. and 50° C., 25° C. and 40° C., 25° C. and 35° C., 25° C. and 30° C., 35° C. and 90° C., 35° C. and 80° C., 35° C. and 70° C., 35° C. and 60° C., 35° C. and 50° C., 35° C. and 40° C., 40° C. and 90° C., 40° C. and 80° C., 40° C. and 70° C., 40° C. and 60° C., and 40° C. and 50° C.

In a further aspect, the resulting fixed tissue sample from any one of the timepoints and/or temperatures described herein is capable of being successfully assayed via a single cell workflow described herein and returning viable data. In some aspects, viable data is not the absence of positive data, rather it is the successful elucidation of the transcriptome of the cells of the tissue sample. In some aspects, a control tissue sample that was not subjected to fixation, but otherwise subject to identical conditions as a tissue sample that was subject to fixation, yields unviable data or a loss of complexity of the types of cells and/or nucleic acid transcripts in the control tissue sample relative to the experimental tissue sample subjected to fixation.

The present disclosure also provides an assay method that comprises the steps of: (a) generating a discrete droplet encapsulating a fixed cell resulting from the chop-fix preparation of a biological tissue, and assay reagents; and (b) detecting analytes from the reaction of the assay reagents and the fixed cells. Optionally, the steps of the method can further comprise including within the discrete droplet generated in step (a) an un-fixing agent, and/or a low-temperature active protease, and then detecting in step (b) analytes from the reaction of the assay reagents and the un-fixed cells

A wide range of partition-based assays and systems are known in the art. Assays and systems that are suitable for use with the present disclosure include, without limitation, those described in U.S. Pat. Nos. 9,694,361, 10,357,771, 10,273,541, and 10,011,872, as well as US Published Patent Application Nos. 20180105808, 20190367982, and 20190338353, each of which is incorporated herein by reference in its entirety. It is contemplated that any assay that can be carried out using a fresh biological sample, such as a single cell/nucleus encapsulated in a droplet with a bead carrying a barcode, can also be carried out using a biological sample prepared using the chop-fix methods of the present disclosure. That is, in any partition-based assay the dissociated fixed cells resulting from the chop-fix method can be used where the protocol comprises encapsulating a cell from the composition of dissociated fixed cells with assay reagents in a discrete droplet, and optionally, also encapsulating together with an un-fixing agent and/or low-temperature active protease.

Exemplary assays include single-cell transcription profiling, single-cell sequence analysis, immune profiling of individual T and B cells, single-cell/single-nucleus chromatin accessibility analysis (e.g., ATAC seq analysis). These exemplary assays can be carried out using commercially available systems for encapsulating biological samples, gel beads, barcodes, and/or other compounds/materials in droplets, such as The Chromium System (10× Genomics, Pleasanton, CA, USA).

In some embodiments of the assay methods, the discrete droplet further comprises one or more beads. In some embodiments, the bead(s) can contain the assay reagents and/or the un-fixing agent. In some embodiments, a barcode is carried by or contained in a bead. Compositions, methods and systems for sample preparation, amplification, and sequencing of biomolecules from single cells encapsulated with barcodes in droplets are provided in e.g., US Pat. Publication No. 20180216162A1, which is hereby incorporated by reference herein.

Assay reagents can include those used to perform one or more additional chemical or biochemical operations on a biological sample encapsulated in a droplet. Accordingly, assay reagents useful in the assay method include any reagents useful in performing a reaction such as nucleic acid modification (e.g., ligation, digestion, methylation, random mutagenesis, bisulfite conversion, uracil hydrolysis, nucleic acid repair, capping, or decapping), nucleic acid amplification (e.g., isothermal amplification or PCR), nucleic acid insertion or cleavage (e.g., via CRISPR/Cas9-mediated or transposon-mediated insertion or cleavage), and/or reverse transcription. Additionally, useful assay reagents can include those that allow the preparation of a target sequence or sequencing reads that are specific to the macromolecular constituents of interest at a higher rate than to non-target sequence specific reads.

In addition, the present disclosure provides compositions and systems related to the analysis of biological samples prepared from tissue with the chop-fix methods. In one embodiment, the present disclosure provides a composition comprising a plurality of partitions, wherein a subset of said plurality of partitions comprises dissociated fixed cells or nuclei, derived from a chop-fix preparation of biological tissue. In another embodiment, a partition of the plurality of partitions comprises a fixed cell/nucleus, an un-fixing agent, and a low-temperature active protease. In certain embodiments, the fixed cell/nucleus is a single fixed cell/nucleus. In other embodiments the present disclosure provides a composition comprising a plurality of partitions, wherein the plurality of partitions comprises a plurality of fixed cells or fixed nuclei derived from a chop-fix preparation of a biological tissue type. In one embodiment, the plurality of partitions each further comprises an un-fixing agent and a low-temperature active protease, as described herein. The partition may be a droplet or a well.

In some embodiments, the partition or partitions described herein may further comprise one or more of the following: a reverse transcriptase (RT), a bead, and reagents for a nucleic acid extension reaction. In at least one embodiment, the protease and/or un-fixing agent compositions can be provided at a temperature other than ambient temperature. In one embodiment, the temperature is below ambient temperature or above ambient temperature.

As described elsewhere herein, partitioning approaches may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions. For example, an occupied partition according the present disclosure comprises a fixed cell/nucleus, and optionally, an un-fixing agent and/or a low-temperature active protease composition.

In another aspect, the present disclosure concerns methods for the partitioning of a plurality of fixed cells or nuclei into individual partitions. In some cases, about 10, about 20, about 30, about 40, about 50, about 60, about 70, about 80, about 90, about 100, about 200, about 300, about 400, about 500, about 600, about 700, about 800, about 900, about 1000, about 2000, about 3000, about 4000, about 5000, about 6000, about 7000, about 8000, about 9000, about 10,000, about 15,000, about 20,000, about 25,000, about 30,000, about 35,000, about 40,000, about 50,000, about 60,000, about 70,000, about 80,000, about 90,000 or about 100,000 fixed cells or nuclei may be partitioned into individual partitions. In some instances, the method further comprises partitioning about 50 to about 20,000 fixed cells or nuclei with each of a plurality of supports comprising the adaptor comprising the barcode sequence, wherein the barcode sequence is unique among each of the plurality of supports.

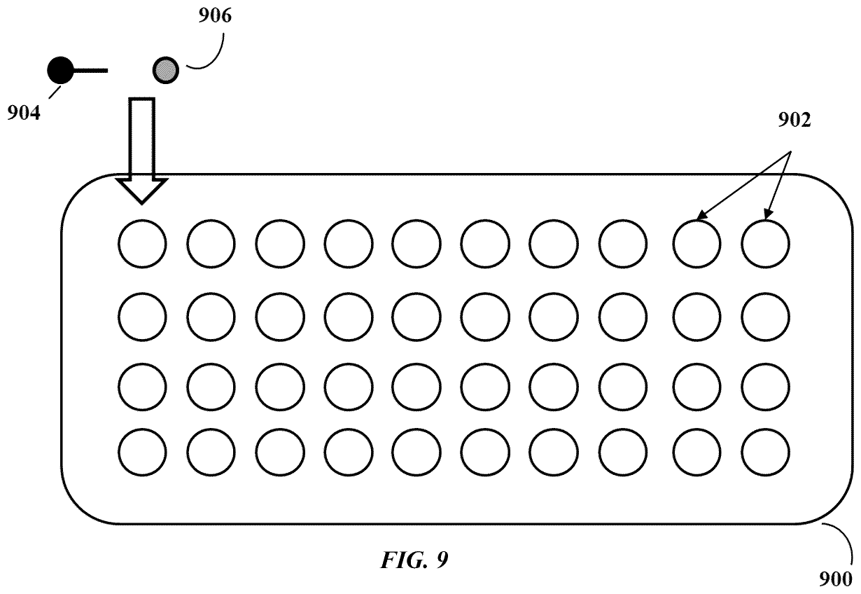

FIG. 9 schematically illustrates an example of a microwell array. The array can be contained within a substrate 900. The substrate 900 comprises a plurality of wells 902. The wells 902 may be of any size or shape, and the spacing between the wells, the number of wells per substrate, as well as the density of the wells on the substrate 900 can be modified, depending on the particular application. In one such example application, a sample molecule 906, which may comprise a cell/nucleus (e.g., a fixed cell/nucleus or an un-fixed cell/nucleus) or cellular components (e.g., nucleic acid molecules) is co-partitioned with a bead 904, which may comprise a nucleic acid barcode molecule coupled thereto. The wells 902 may be loaded using gravity or other loading technique (e.g., centrifugation, liquid handler, acoustic loading, optoelectronic, etc.). In some instances, at least one of the wells 902 contains a single sample molecule 906 (e.g., cell or nucleus) and a single bead 904.

Reagents may be loaded into a well either sequentially or concurrently. In some cases, reagents are introduced to the device either before or after a particular operation. In some cases, reagents (which may be provided, in certain instances, in droplets or beads) are introduced sequentially such that different reactions or operations occur at different steps. The reagents (or droplets or beads) may also be loaded at operations interspersed with a reaction or operation step. For example, droplets or beads comprising reagents for fragmenting polynucleotides (e.g., restriction enzymes) and/or other enzymes (e.g., transposases, ligases, polymerases, etc.) may be loaded into the well or plurality of wells, followed by loading of droplets or beads comprising reagents for attaching nucleic acid barcode molecules to a sample nucleic acid molecule. Reagents may be provided concurrently or sequentially with a sample, such as a cell/nucleus (e.g., a fixed cell/nucleus or an un-fixed cell/nucleus) or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a bead or droplet. These beads or droplets may be loaded into a partition (e.g., a microwell) before, after, or concurrently with the loading of a cell/nucleus (e.g., a fixed cell/nucleus or an un-fixed cell/nucleus), such that each cell or nucleus is contacted with a different bead or droplet. This technique may be used to attach a unique nucleic acid barcode molecule to nucleic acid molecules obtained from each cell/nucleus (e.g., a fixed cell/nucleus or an un-fixed cell/nucleus). Alternatively, or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell/nucleus or partition, while polynucleotides with different barcodes may be determined to originate from different cells/nuclei or partitions.

The samples or reagents may be loaded in the wells or microwells using a variety of approaches. The samples (e.g., a cell, nucleus, or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell/nucleus, e.g., a single fixed cell/nucleus or a single un-fixed cell/nucleus) and a single bead (such as those described herein, which may, in some instances, also be provided or encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where cells/nuclei, e.g., fixed cells/nuclei or un-fixed cells/nuclei are loaded in the microwells, one of the droplets may comprise reagents for further processing, e.g., lysis reagents for lysing the cell/nucleus, upon droplet merging.

A droplet may be partitioned into a well. The droplets may be selected or subjected to pre-processing prior to loading into a well. For instance, the droplets may comprise cells, e.g., fixed cells or un-fixed cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell/nucleus (e.g., a fixed cell/nucleus or an un-fixed cell/nucleus) distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. For instance, the nucleic acid barcode molecules may comprise a chemical crosslinker which may be cleaved upon application of a stimulus (e.g., photo-, magnetic, chemical, biological, stimulus). The released nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In such cases, the unique partition barcode sequences may be used to identify the cell/nucleus or partition from which a nucleic acid molecule originated.

Characterization of samples within a well may be performed. Such characterization can include, in non-limiting examples, imaging of the sample (e.g., cell, nucleus, or cellular components) or derivatives thereof. Characterization techniques such as microscopy or imaging may be useful in measuring sample profiles in fixed spatial locations. For instance, when cells/nuclei (e.g., fixed cells/nuclei or un-fixed cells/nuclei) are partitioned, optionally with beads, imaging of each microwell and the contents contained therein may provide useful information on cell/nucleus doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, cell-cell interactions (when two or more cells are co-partitioned). Alternatively, or in addition to, imaging may be used to characterize a quantity of amplification products in the well.

In operation, a well may be loaded with a sample and reagents, simultaneously or sequentially. When cells/nuclei (e.g., fixed cells/nuclei or un-fixed cells/nuclei) are loaded, the well may be subjected to washing, e.g., to remove excess cells/nuclei from the well, microwell array, or plate. Similarly, washing may be performed to remove excess beads or other reagents from the well, microwell array, or plate. In addition, the cells/nuclei may be lysed in the individual partitions to release the intracellular/intranuclear components or cellular analytes. Alternatively, the cells/nuclei may be fixed or permeabilized in the individual partitions. The intracellular components or cellular analytes may couple to a support, e.g., on a surface of the microwell, on a solid support (e.g., bead), or they may be collected for further downstream processing. For instance, after cell/nucleus lysis, the intracellular/intranuclear components or cellular analytes may be transferred to individual droplets or other partitions for barcoding. Alternatively, or in addition to, the intracellular/intranuclear components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular/intranuclear components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

Once sealed, the well may be subjected to conditions for further processing of a cell/nucleus (or cells/nuclei) in the well. For instance, reagents in the well may allow further processing of the cell/nucleus, e.g., cell lysis, as further described herein. Alternatively, the well (or wells such as those of a well-based array) comprising the cell/nucleus (or cells/nuclei) may be subjected to freeze-thaw cycling to process the cell/nucleus (or cells/nuclei), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell/nucleus (or cells/nuclei) may be subjected to freeze thaw cycles to lyse the cell/nucleus (or cells/nuclei). In one embodiment, the initially frozen well (or wells) are thawed to a temperature above freezing (e.g., room temperature or 25° C.). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, or 4 times, to obtain lysis of the cell/nucleus (or cells/nuclei) in the well (or wells). In one embodiment, the freezing, thawing and/or freeze/thaw cycling is performed in the absence of a lysis buffer.

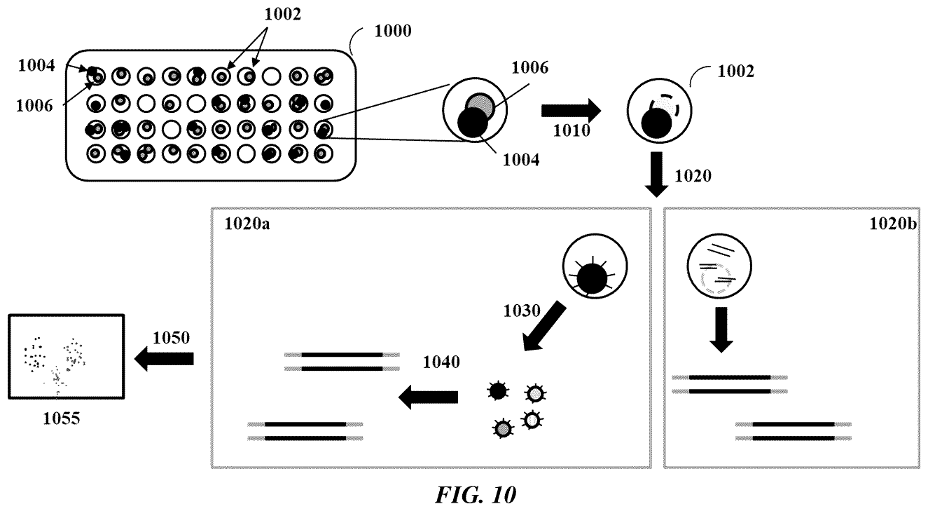

FIG. 10 schematically shows an example workflow for processing nucleic acid molecules within a sample. A substrate 1000 comprising a plurality of microwells 1002 may be provided. A sample 1006 which may comprise a cell/nucleus (e.g., a fixed cell/nucleus or an un-fixed cell/nucleus), cellular components or analytes (e.g., proteins and/or nucleic acid molecules) can be co-partitioned, in a plurality of microwells 1002, with a plurality of beads 1004 comprising nucleic acid barcode molecules. During process 1010, the sample 1006 may be processed within the partition. For instance, the cell may be subjected to conditions sufficient to lyse the cells/nuclei (e.g., fixed cells/nuclei or un-fixed cells/nuclei) and release the analytes contained therein. In process 1020, the bead 1004 may be further processed. By way of example, processes 1020a and 1020b schematically illustrate different workflows, depending on the properties of the bead 1004.

In 1020a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 1030, the beads 1004 from multiple wells 1002 may be collected and pooled. Further processing may be performed in process 1040. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1050, further characterization, such as sequencing may be performed to generate sequencing reads. The sequencing reads may yield information on individual cells/nuclei or populations of cells/nuclei (e.g., fixed cells/nuclei or un-fixed cells/nuclei), which may be represented visually or graphically, e.g., in a plot 1055.

In 1020b, the bead comprises nucleic acid barcode molecules that are releasably attached thereto, as described below. The bead may degrade or otherwise release the nucleic acid barcode molecules into the well 1002; the nucleic acid barcode molecules may then be used to barcode nucleic acid molecules within the well 1002. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1050, further characterization, such as sequencing may be performed to generate sequencing reads. The sequencing reads may yield information on individual cells/nuclei or populations of cells/nuclei (e.g., fixed cells/nuclei or un-fixed cells/nuclei), which may be represented visually or graphically, e.g., in a plot 1055.

### G. Additional Methods

The present disclosure provides methods and systems for multiplexing, and otherwise increasing throughput of samples (e.g., cells/nuclei, fixed cells/nuclei, or un-fixed cells/nuclei) for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cells/nuclei (e.g., cells/nuclei, fixed cells/nuclei or un-fixed cells/nuclei) or cell/nuclear features may be used to characterize cells/nuclei and/or cell/nucleus features. In some instances, cell features include cell surface features and the nucleus features include nuclear membrane features. Cell surface or nuclear membrane features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell/nucleus features may include intracellular/intranuclear analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface/nuclear membrane feature to which the binding group binds. For example, the reporter oligonucleotide may comprise a barcode sequence (e.g., a reporter sequence) that permits identification of the labelling agent. For example, a labelling agent that is specific to one type of cell/nucleus feature (e.g., a first cell surface feature or first nuclear membrane feature) may have a first reporter oligonucleotide coupled thereto, while a labelling agent that is specific to a different cell/nucleus feature (e.g., a second cell surface feature or first nuclear membrane feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, each of which is herein entirely incorporated by reference for all purposes.

In a particular example, a library of potential cell/nuclear feature labelling agents may be provided, where the respective cell/nucleus feature labelling agents are associated with nucleic acid reporter molecules, such that a different reporter oligonucleotide sequence is associated with each labelling agent capable of binding to a specific cell/nucleus feature. In other aspects, different members of the library may be characterized by the presence of a different oligonucleotide sequence label. For example, an antibody capable of binding to a first protein may have associated with it a first reporter oligonucleotide sequence, while an antibody capable of binding to a second protein may have a different reporter oligonucleotide sequence associated with it. The presence of the particular oligonucleotide sequence may be indicative of the presence of a particular antibody or cell/nucleus feature which may be recognized or bound by the particular antibody.

For workflows comprising the use of fixation agents and/or un-fixing agents, labelling agents may be used to label samples (e.g., cells/nuclei, fixed cells/nuclei, or un-fixed cells/nuclei) at different points in time. In one embodiment, a plurality of cells/nuclei is labeled prior to treatment with a fixation agent and/or after treatment with a fixation agent. In another embodiment, a plurality of fixed cells/nuclei is labeled prior to treatment with an un-fixing agent and/or after treatment with an un-fixing agent. In one additional embodiment, a plurality of un-fixed cells/nuclei is labeled prior to partitioning into partitions (e.g., wells or droplets) for further processing. In another embodiment, the methods, compositions, systems, and kits described herein provide labeled cells/nuclei, labeled fixed cells/nuclei, or labeled un-fixed cells/nuclei.

Labelling agents capable of binding to or otherwise coupling to one or more cells/nuclei may be used to characterize a cell/nucleus as belonging to a particular set of cells/nuclei. For example, labeling agents may be used to label a sample of cells/nuclei or a group of cells/nuclei. In this way, a group of cells/nuclei may be labeled as different from another group of cells/nuclei. In an example, a first group of cells/nuclei may originate from a first sample and a second group of cells/nuclei may originate from a second sample. Labelling agents may allow the first group and second group to have a different labeling agent (or reporter oligonucleotide associated with the labeling agent). This may, for example, facilitate multiplexing, where cells/nuclei of the first group and cells/nuclei of the second group may be labeled separately and then pooled together for downstream analysis. The downstream detection of a label may indicate analytes as belonging to a particular group.

For example, a reporter oligonucleotide may be linked to an antibody or an epitope binding fragment thereof, and labeling a cell/nucleus may comprise subjecting the antibody-linked barcode molecule or the epitope binding fragment-linked barcode molecule to conditions suitable for binding the antibody to a molecule present on a surface of the cell/nucleus. The binding affinity between the antibody or the epitope binding fragment thereof and the molecule present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds may be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 μM, 800 μM, 700 μM, 600 μM, 500 μM, 400 μM, 300 μM, 200 μM, 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, or 1 μM. For example, the dissociation constant may be less than about 10 μM.

In another example, a reporter oligonucleotide may be coupled to a cell-penetrating peptide (CPP), and labeling cells/nuclei may comprise delivering the CPP coupled reporter oligonucleotide into an analyte carrier. Labeling analyte carriers may comprise delivering the CPP conjugated oligonucleotide into a cell or nucleus by the cell-penetrating peptide. A CPP that can be used in the methods provided herein can comprise at least one non-functional cysteine residue, which may be either free or derivatized to form a disulfide link with an oligonucleotide that has been modified for such linkage. Non-limiting examples of CPPs that can be used in embodiments herein include penetratin, transportan, plsl, TAT (48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells/nuclei of a cell/nucleus population. The CPP may be an arginine-rich peptide transporter. The CPP may be Penetratin or the Tat peptide. In another example, a reporter oligonucleotide may be coupled to a fluorophore or dye, and labeling cells/nuclei may comprise subjecting the fluorophore-linked barcode molecule to conditions suitable for binding the fluorophore to the surface of the cell or the nuclear membrane of a nucleus. In some instances, fluorophores can interact strongly with lipid bilayers and labeling cells/nuclei may comprise subjecting the fluorophore-linked barcode molecule to conditions such that the fluorophore binds to or is inserted into a membrane of the cell or nucleus. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649, which is hereby incorporated by reference in its entirety for all purposes, for a description of organic fluorophores.

A reporter oligonucleotide may be coupled to a lipophilic molecule, and labeling cells/nuclei may comprise delivering the nucleic acid barcode molecule to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane may be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide may enter into the intracellular space and/or a cell nucleus. In one embodiment, a reporter oligonucleotide coupled to a lipophilic molecule will remain associated with and/or inserted into lipid membrane (as described herein) via the lipophilic molecule until lysis of the cell/nucleus occurs, e.g., inside a partition.

A reporter oligonucleotide may be part of a nucleic acid molecule comprising any number of functional sequences, as described elsewhere herein, such as a target capture sequence, a random primer sequence, and the like, and coupled to another nucleic acid molecule that is, or is derived from, the analyte.

Prior to partitioning, the cells/nuclei may be incubated with the library of labelling agents, that may be labelling agents to a broad panel of different cell/nucleus features, e.g., receptors, proteins, etc., and which include their associated reporter oligonucleotides. Unbound labelling agents may be washed from the cells/nuclei, and the cells/nuclei may then be co-partitioned (e.g., into droplets or wells) along with partition-specific barcode oligonucleotides (e.g., attached to a support, such as a bead or gel bead) as described elsewhere herein. As a result, the partitions may include the cell/nucleus or cells/nuclei, as well as the bound labelling agents and their known, associated reporter oligonucleotides.

In other instances, e.g., to facilitate sample multiplexing, a labelling agent that is specific to a particular cell/nucleus feature may have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent may interact with different cells/nuclei, cell/nucleus populations or samples, allowing a particular report oligonucleotide to indicate a particular cell/nucleus population (or cell/nucleus or sample) and cell/nuclear feature. In this way, different samples or groups can be independently processed and subsequently combined together for pooled analysis (e.g., partition-based barcoding as described elsewhere herein). See, e.g., U.S. Pat. Pub. 20190323088, which is hereby entirely incorporated by reference for all purposes.

As described elsewhere herein, libraries of labelling agents may be associated with a particular cell/nucleus feature as well as be used to identify analytes as originating from a particular cell/nucleus population, or sample. Cell/nucleus populations may be incubated with a plurality of libraries such that a cell/nucleus or cells/nuclei comprise multiple labelling agents. For example, a cell/nucleus may comprise coupled thereto a lipophilic labeling agent and an antibody. The lipophilic labeling agent may indicate that the cell/nucleus is a member of a particular cell sample, whereas the antibody may indicate that the cell/nucleus comprises a particular analyte. In this manner, the reporter oligonucleotides and labelling agents may allow multi-analyte, multiplexed analyses to be performed.

In some instances, these reporter oligonucleotides may comprise nucleic acid barcode sequences that permit identification of the labelling agent which the reporter oligonucleotide is coupled to. The use of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent, or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide may be allowed to hybridize to the reporter oligonucleotide.

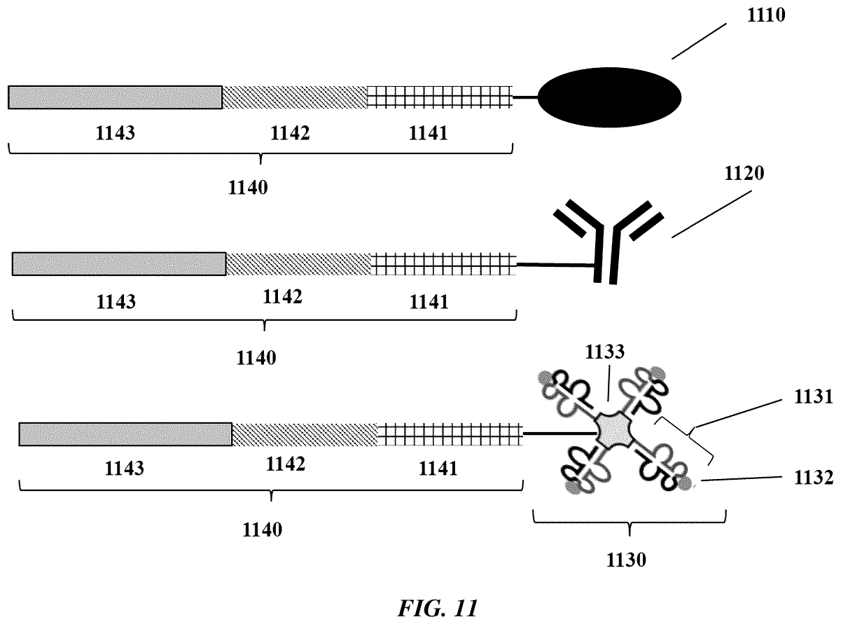

FIG. 11 describes exemplary labelling agents (1110, 1120, 1130) comprising reporter oligonucleotides (1140) attached thereto. Labelling agent 1110 (e.g., any of the labelling agents described herein) is attached (either directly, e.g., covalently attached, or indirectly) to reporter oligonucleotide 1140. Reporter oligonucleotide 1140 may comprise barcode sequence 1142 that identifies labelling agent 1110. Reporter oligonucleotide 1140 may also comprise one or more functional sequences 1143 that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 11, in some instances, reporter oligonucleotide 1140 conjugated to a labelling agent (e.g., 1110, 1120, 1130) comprises a primer sequence 1141, a barcode sequence 1142 that identifies the labelling agent (e.g., 1110, 1120, 1130), and functional sequence 1143. Functional sequence 1143 may be configured to hybridize to a complementary sequence, such as a complementary sequence present on a nucleic acid barcode molecule 1190 (not shown), such as those described elsewhere herein. In some instances, nucleic acid barcode molecule 1190 is attached to a support (e.g., a bead, such as a gel bead), such as those described elsewhere herein. For example, nucleic acid barcode molecule 1190 may be attached to the support via a releasable linkage (e.g., comprising a labile bond), such as those described elsewhere herein. In some instances, reporter oligonucleotide 1140 comprises one or more additional functional sequences, such as those described above.

In some instances, the labelling agent 1110 is a protein or polypeptide (e.g., an antigen or prospective antigen) comprising reporter oligonucleotide 1140. Reporter oligonucleotide 1140 comprises barcode sequence 1142 that identifies polypeptide 1110 and can be used to infer the presence of an analyte, e.g., a binding partner of polypeptide 1110 (i.e., a molecule or compound to which polypeptide 1110 can bind). In some instances, the labelling agent 1110 is a lipophilic moiety (e.g., cholesterol) comprising reporter oligonucleotide 1140, where the lipophilic moiety is selected such that labelling agent 1110 integrates into a membrane of a cell or nucleus. Reporter oligonucleotide 1140 comprises barcode sequence 1142 that identifies lipophilic moiety 1110 which in some instances is used to tag cells/nuclei (e.g., groups of cells/nuclei, cell samples, etc.) and may be used for multiplex analyses as described elsewhere herein. In some instances, the labelling agent is an antibody 1120 (or an epitope binding fragment thereof) comprising reporter oligonucleotide 1140. Reporter oligonucleotide 1140 comprises barcode sequence 1142 that identifies antibody 1120 and can be used to infer the presence of, e.g., a target of antibody 1120 (i.e., a molecule or compound to which antibody 1120 binds). In other embodiments, labelling agent 1130 comprises an MHC molecule 1131 comprising peptide 1132 and reporter oligonucleotide 1140 that identifies peptide 1132. In some instances, the MHC molecule is coupled to a support 1133. In some instances, support 1133 may be a polypeptide, such as streptavidin, or a polysaccharide, such as dextran. In some instances, reporter oligonucleotide 1140 may be directly or indirectly coupled to MHC labelling agent 1130 in any suitable manner. For example, reporter oligonucleotide 1140 may be coupled to MHC molecule 1131, support 1133, or peptide 1132. In some embodiments, labelling agent 1130 comprises a plurality of MHC molecules, (e.g., is an MHC multimer, which may be coupled to a support (e.g., 1133)). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. Pub. 20190367969, each of which is herein entirely incorporated by reference for all purposes.

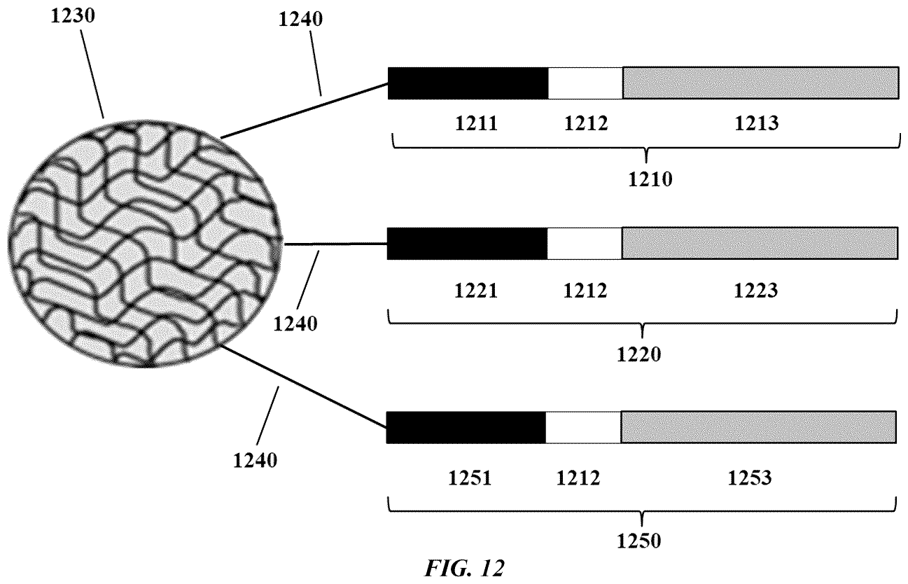

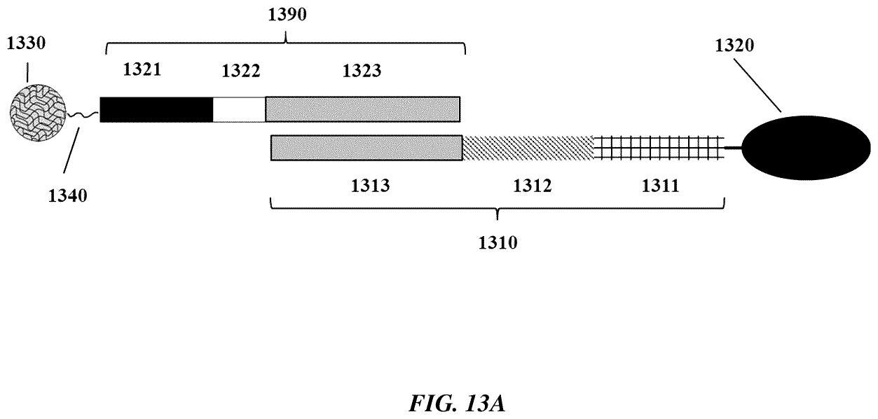

FIG. 12 illustrates another example of a barcode carrying bead. In some embodiments, analysis of multiple analytes (e.g., RNA and one or more analytes using labelling agents described herein) may comprise nucleic acid barcode molecules as generally depicted in FIG. 12. In some embodiments, nucleic acid barcode molecules 1210 and 1212 are attached to support 1230 via a releasable linkage 1240 (e.g., comprising a labile bond) as described elsewhere herein. Nucleic acid barcode molecule 1210 may comprise adapter sequence 1211, barcode sequence 1212 and adapter sequence 1213. Nucleic acid barcode molecule 1220 may comprise adapter sequence 1221, barcode sequence 1212, and adapter sequence 1223, wherein adapter sequence 1223 comprises a different sequence than adapter sequence 1213. In some instances, adapter 1211 and adapter 1221 comprise the same sequence. In some instances, adapter 1211 and adapter 1221 comprise different sequences. Although support 1230 is shown comprising nucleic acid barcode molecules 1210 and 1220, any suitable number of barcode molecules comprising common barcode sequence 1212 are contemplated herein. For example, in some embodiments, support 1230 further comprises nucleic acid barcode molecule 1250. Nucleic acid barcode molecule 1250 may comprise adapter sequence 1251, barcode sequence 1212 and adapter sequence 1253, wherein adapter sequence 1253 comprises a different sequence than adapter sequence 1213 and 1223. In some instances, nucleic acid barcode molecules (e.g., 1210, 1220, 1250) comprise one or more additional functional sequences, such as a UMI or other sequences described herein. The nucleic acid barcode molecules 1210, 1220 or 1250 may interact with analytes as described elsewhere herein, for example, as depicted in FIGS. 13A-C.

Referring to FIG. 13A, in an instance where cells/nuclei are labelled with labeling agents, sequence 1323 may be complementary to an adapter sequence of a reporter oligonucleotide. Cells/nuclei may be contacted with one or more reporter oligonucleotide 1310 conjugated labelling agents 1320 (e.g., polypeptide, antibody, or others described elsewhere herein). In some cases, the cells/nuclei may be further processed prior to barcoding. For example, such processing steps may include one or more washing and/or cell sorting steps. In some instances, a cell/nucleus that is bound to labelling agent 1320 which is conjugated to oligonucleotide 1310 and support 1330 (e.g., a bead, such as a gel bead) comprising nucleic acid barcode molecule 1390 is partitioned into a partition amongst a plurality of partitions (e.g., a droplet of a droplet emulsion or a well of a microwell array). In some instances, the partition comprises at most a single cell/nucleus bound to labelling agent 1320. In some instances, reporter oligonucleotide 1310 conjugated to labelling agent 1320 (e.g., polypeptide, an antibody, pMHC molecule such as an MHC multimer, etc.) comprises a first adapter sequence 1311 (e.g., a primer sequence), a barcode sequence 1312 that identifies the labelling agent 1320 (e.g., the polypeptide, antibody, or peptide of a pMHC molecule or complex), and an adapter sequence 1313. Adapter sequence 1313 may be configured to hybridize to a complementary sequence, such as sequence 1323 present on a nucleic acid barcode molecule 1390. In some instances, oligonucleotide 1310 comprises one or more additional functional sequences, such as those described elsewhere herein.

Barcoded nucleic may be generated (e.g., via a nucleic acid reaction, such as nucleic acid extension or ligation) from the constructs described in FIGS. 13A-C. For example, sequence 1313 may then be hybridized to complementary sequence 1323 to generate (e.g., via a nucleic acid reaction, such as nucleic acid extension or ligation) a barcoded nucleic acid molecule comprising cell/nucleus (e.g., partition specific) barcode sequence 1321 (or a reverse complement thereof) and reporter sequence 1312 (or a reverse complement thereof). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. For example, the workflow may comprise a workflow as generally depicted in any of FIGS. 13A-C, or a combination of workflows for an individual analyte, as described elsewhere herein. For example, by using a combination of the workflows as generally depicted in FIGS. 13A-C, multiple analytes can be analyzed.

In some instances, analysis of an analyte (e.g. a nucleic acid, a polypeptide, a carbohydrate, a lipid, etc.) comprises a workflow as generally depicted in FIG. 13A. A nucleic acid barcode molecule 1390 may be co-partitioned with the one or more analytes. In some instances, nucleic acid barcode molecule 1390 is attached to a support 1330 (e.g., a bead, such as a gel bead), such as those described elsewhere herein. For example, nucleic acid barcode molecule 1390 may be attached to support 1330 via a releasable linkage 1340 (e.g., comprising a labile bond), such as those described elsewhere herein. Nucleic acid barcode molecule 1390 may comprise a barcode sequence 1321 and optionally comprise other additional sequences, for example, a UMI sequence 1322 (or other functional sequences described elsewhere herein). The nucleic acid barcode molecule 1390 may comprise a sequence 1323 that may be complementary to another nucleic acid sequence, such that it may hybridize to a particular sequence.

For example, sequence 1323 may comprise a poly-T sequence and may be used to hybridize to mRNA. Referring to FIG. 13C, in some embodiments, nucleic acid barcode molecule 1390 comprises sequence 1323 complementary to a sequence of RNA molecule 1360 from a cell/nucleus. In some instances, sequence 1323 comprises a sequence specific for an RNA molecule. Sequence 1323 may comprise a known or targeted sequence or a random sequence. In some instances, a nucleic acid extension reaction may be performed, thereby generating a barcoded nucleic acid product comprising sequence 1323, the barcode sequence 1321, UMI sequence 1322, any other functional sequence, and a sequence corresponding to the RNA molecule 1360.

In another example, sequence 1323 may be complementary to an overhang sequence or an adapter sequence that has been appended to an analyte. For example, referring to FIG. 13B, in some embodiments, primer 1350 comprises a sequence complementary to a sequence of nucleic acid molecule 1360 (such as an RNA encoding for a BCR sequence) from an analyte carrier. In some instances, primer 1350 comprises one or more sequences 1351 that are not complementary to RNA molecule 1360. Sequence 1351 may be a functional sequence as described elsewhere herein, for example, an adapter sequence, a sequencing primer sequence, or a sequence the facilitates coupling to a flow cell of a sequencer. In some instances, primer 1350 comprises a poly-T sequence. In some instances, primer 1350 comprises a sequence complementary to a target sequence in an RNA molecule. In some instances, primer 1350 comprises a sequence complementary to a region of an immune molecule, such as the constant region of a TCR or BCR sequence. Primer 1350 is hybridized to nucleic acid molecule 1360 and complementary molecule 1370 is generated. For example, complementary molecule 1370 may be cDNA generated in a reverse transcription reaction. In some instances, an additional sequence may be appended to complementary molecule 1370. For example, the reverse transcriptase enzyme may be selected such that several non-templated bases 1380 (e.g., a poly-C sequence) are appended to the cDNA. In another example, a terminal transferase may also be used to append the additional sequence. Nucleic acid barcode molecule 1390 comprises a sequence 1324 complementary to the non-templated bases, and the reverse transcriptase performs a template switching reaction onto nucleic acid barcode molecule 1390 to generate a barcoded nucleic acid molecule comprising cell/nucleus (e.g., partition specific) barcode sequence 1322 (or a reverse complement thereof) and a sequence of complementary molecule 1370 (or a portion thereof). In some instances, sequence 1323 comprises a sequence complementary to a region of an immune molecule, such as the constant region of a TCR or BCR sequence. Sequence 1323 is hybridized to nucleic acid molecule 1360 and a complementary molecule 1370 is generated. For example, complementary molecule 1370 may be generated in a reverse transcription reaction generating a barcoded nucleic acid molecule comprising cell/nucleus (e.g., partition specific) barcode sequence 1322 (or a reverse complement thereof) and a sequence of complementary molecule 1370 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969, each of which applications is herein entirely incorporated by reference for all purposes.

### Single Cell/Single Nucleus ATAC-Seq

The sample preparation methods, compositions, systems, and kits disclosed herein may be used for processing multiple different types of nucleic acid molecules on an individual basis or in tandem from single cells or nuclei, e.g., suspensions of single cells or nuclei derived from different sample types including liquid and solid tissue samples. The methods provided herein may allow for analysis of various deoxyribonucleic acid (DNA) molecules and/or ribonucleic acid (RNA) molecules from the same biological particle (e.g., cell or cellular component such as a cell nucleus). DNA and/or RNA molecules may originate from the biological particle. Analysis of different types of nucleic acid molecules may be performed simultaneously or near simultaneously. The methods provided herein may comprise use of a partitioning scheme in which materials (e.g., different types of target nucleic acid molecules, such as target nucleic acid molecules included within a cell or nucleus) are distributed between a plurality of partitions, such as a plurality of droplets or wells. Materials (e.g., target nucleic acid molecules) may be co-partitioned with one or more reagents in order to generate a barcoded nucleic acid product (e.g., within a partition of a plurality of partitions) corresponding to each of various different target nucleic acid molecules (e.g., DNA and RNA molecules), prior to subjecting the barcoded nucleic acid products to one or more amplification processes (e.g., polymerase chain reaction (PCR), which may optionally be performed in bulk).

Biological particles (e.g., cells or cell nuclei) may be pre-sorted based on a transduced marker prior to analysis (e.g., as described herein) by fluorescence-activated cell sorting (FACS) or magnetic-activated cell sorting (MACS). For example, cells may be transduced with a fluorescent protein (e.g., GFP, YFP, CFP, mCherry, mRuby, etc.).

Accordingly, the methods provided herein provide sample preparation techniques that permit sequencing of nucleic acid molecules from single cells and nuclei of interest. In eukaryotic genomes, chromosomal DNA winds itself around histone proteins (i.e., “nucleosomes”), thereby forming a complex known as chromatin. The tight or loose packaging of chromatin contributes to the control of gene expression. Tightly packed chromatin (“closed chromatin”) is usually not permissive for gene expression while more loosely packaged, accessible regions of chromatin (“open chromatin”) is associated with the active transcription of gene products. Methods for probing genome-wide DNA accessibility have proven extremely effective in identifying regulatory elements across a variety of cell types and in quantifying changes that lead to activation and/or repression of gene expression. One such method is the Assay for Transposase Accessible Chromatin with high-throughput sequencing (ATAC-seq). The ATAC-seq method probes DNA accessibility with an artificial transposon, which inserts specific sequences into accessible regions of chromatin. Because the transposase can only insert sequences into accessible regions of chromatin not bound by transcription factors and/or nucleosomes, sequencing reads can be used to infer regions of increased chromatin accessibility. Single cell ATAC-seq (scATAC-seq) methods have been developed, as described in PCT Patent Publications Nos. WO2018/218226, U.S. Pat. Nos. 10,844,372 and 10,725,027, and US. Pat. Pub. 20200291454, each of which is incorporated by reference in its entirety.

The methods, compositions, systems, and kits of the present disclosure may be used to prepare samples for downstream analysis with the Assay for Transposase Accessible Chromatin with high-throughput sequencing (ATAC-seq) and RNA sequencing (RNA-seq) assays alone or in tandem. Such analysis may reveal regulatory factors that control cis-element accessibility and/or trans-factor occupancy. In some cases, the methods, systems, and kits provided herein may reveal nucleosome positioning and/or regulatory nodules of coordinated activity in a cell type, such as coordinated trans-factor activities, synergistic activities of co-binding transcription factors (TFs) on cis-elements, etc. The methods provided herein may be performed in a high throughput manner to facilitate obtaining single biological particle (e.g., single cell or single nucleus) data, including epigenomic variability.

In some embodiments, the biological particle is a cell or cell nucleus. In some embodiments, the cell or the cell nucleus is permeabilized. In some embodiments, the method further comprises lysing or permeabilizing the biological particle within the partition to provide access to the DNA molecule and/or the RNA molecule therein. In some embodiments, the method further comprises processing an open chromatin structure of the biological particle with a transposase to yield the DNA molecule.

A nucleic acid molecule may undergo one or more processing steps within a biological particle (e.g., cell or cell nucleus). For example, chromatin within a cell or cell nucleus may be contacted with a transposase. Examples of processing steps including a transposon complex may be found in, for example, US. Pat. Pub. 20180340171 and US. Pat. Pub. 20200291454, each of which are herein incorporated by reference in their entireties.

In an aspect, the present disclosure provides sample preparation techniques suitable for use in methods for processing DNA and/or RNA nucleic acid molecules from a cell or cell nucleus. The method may comprise contacting a cell or cell nucleus with a transposase-nucleic acid complex comprising a transposase molecule and one or more transposon end oligonucleotide molecules. The cell or cell nucleus may be contacted with a transposase-nucleic acid complex in bulk solution, such that the cell or cell nucleus undergoes “tagmentation” via a tagmentation reaction. Contacting the cell or cell nucleus with the transposase-nucleic acid complex may generate one or more template nucleic acid fragments (e.g., “tagmented fragments”). The one or more template nucleic acid fragments may correspond to one or more target nucleic acid molecules (e.g., deoxyribonucleic acid (DNA) molecules) within the cell or cell nucleus. In parallel, the cell or cell nucleus may be contacted with a primer molecule (e.g., a primer molecule comprising a poly-T sequence) configured to interact with one or more additional target nucleic acid molecules (e.g., ribonucleic acid (RNA) molecules, such as messenger RNA (mRNA) molecules). The cell or cell nucleus may be contacted with a primer molecule in bulk solution. Alternatively, or in addition to, the cell or cell nucleus may be contacted with a primer molecule within a partition. Interaction between these moieties may yield one or more additional template nucleic acid fragments (e.g., RNA fragments). For example, the primer molecule may have at least partial sequence complementarity to the one or more additional target nucleic acid molecules (e.g., mRNA molecules). The primer molecule may hybridize to a sequence of an additional target nucleic acid molecule of the one or more additional target nucleic acid molecules. The cell or cell nucleus may be partitioned (e.g., co-partitioned with one or more reagents) into a partition (e.g., of a plurality of partitions). The partition may be, for example, a droplet or a well. The partition may comprise one or more reagents, including, for example, one or more particles (e.g., beads) comprising one or more nucleic acid barcode molecules. The cell or cell nucleus may be lysed, permeabilized, fixed, crosslinked, or otherwise manipulated to provide access to the one or more template nucleic acid fragments and the one or more additional template nucleic acid fragments therein. The one or more template nucleic acid fragments and the one or more additional template nucleic acid fragments therein may undergo one or more processing steps within the partition. For example, the one or more template nucleic acid fragments and/or the one or more additional template nucleic acid fragments may undergo a barcoding process, a ligation process, a reverse transcription process, a template switching process, a linear amplification process, and/or a gap filling process. The resultant one or more processed template nucleic acid fragments (e.g., tagmented fragments) and/or the one or more processed additional template nucleic acid fragments (e.g., RNA fragments) may each include a barcode sequence (e.g., nucleic acid barcode sequence, as described herein). The one or more processed template nucleic acid fragments and/or the one or more processed additional template nucleic acid fragments may be released from the partition (e.g., pooled with contents of other partitions of a plurality of partitions) and may undergo one or more additional processing steps in bulk. For example, the one or more processed template nucleic acid fragments and/or the one or more processed additional template nucleic acid fragments may undergo a gap filling process, a dA tailing process, a terminal-transferase process, a phosphorylation process, a ligation process, a nucleic acid amplification process, or a combination thereof. For example, the one or more processed template nucleic acid fragments and/or the one or more processed additional template nucleic acid fragments may be subjected to conditions sufficient to undergo one or more polymerase chain reactions (PCR, such as sequence independent PCR) to generate amplification products corresponding to the one or more processed template nucleic acid fragments (e.g., tagmented fragments) and/or the one or more processed additional template nucleic acid fragments (e.g., RNA fragments). Sequences of such amplification products can be detected using, for example, a nucleic acid sequencing assay and used to identify sequences of the one or more target nucleic acid molecules (e.g., DNA molecules) and the one or more additional target nucleic acid molecules (e.g., RNA molecules) of the cell or cell nucleus from which they derive. Additional methods, compositions, systems, and kits for processing DNA and/or RNA nucleic acid molecules from a cell or cell nucleus are disclosed in U.S. Pat. Pub. No. 20200291454A1, which is herein incorporated by reference in its entirety.

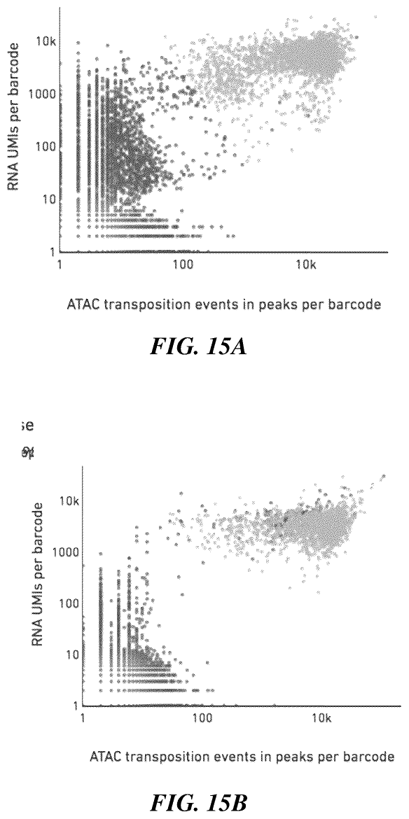

A nucleic acid barcode molecule of the one or more nucleic acid barcode molecules may comprise a flow cell adapter sequence, a barcode sequence, and a sequencing primer or portion thereof, which sequencing primer or portion thereof may be configured to interact with (e.g., anneal or hybridize to) a complementary sequence included in template nucleic acid fragments deriving from DNA molecules of the biological particle (e.g., cell or cell nucleus), or derivatives thereof (e.g., ATAC-seq fragments generated using, e.g., the composition of FIGS. 17-19).

FIG. 17 includes an example of a transposase-nucleic acid complex for use in the methods provided herein. Transposase-nucleic acid complex 1700 (e.g., comprising a transpose dimer) comprises partially double-stranded oligonucleotide 1701 and partially double-stranded oligonucleotide 1705. Partially double-stranded oligonucleotide 1701 comprises transposon end sequence 1703, first primer sequence 1702, and a sequence 1704 that is complementary to transposon end sequence 1703. Partially double-stranded oligonucleotide 1705 comprises transposon end sequence 1706, first primer sequence 1707, and a sequence 1708 that is complementary to transposon end sequence 1706. Primer sequences 1702 and 1707 may be the same or different. In some cases, primer sequence 1702 may be designated as a first sequencing primer, such as an Illumina “R1” sequence and primer sequence 1707 may be designated as a second sequencing primer, such as an Illumina “R2” sequence. Transposon end sequences 1703 and 1706 may be the same or different.

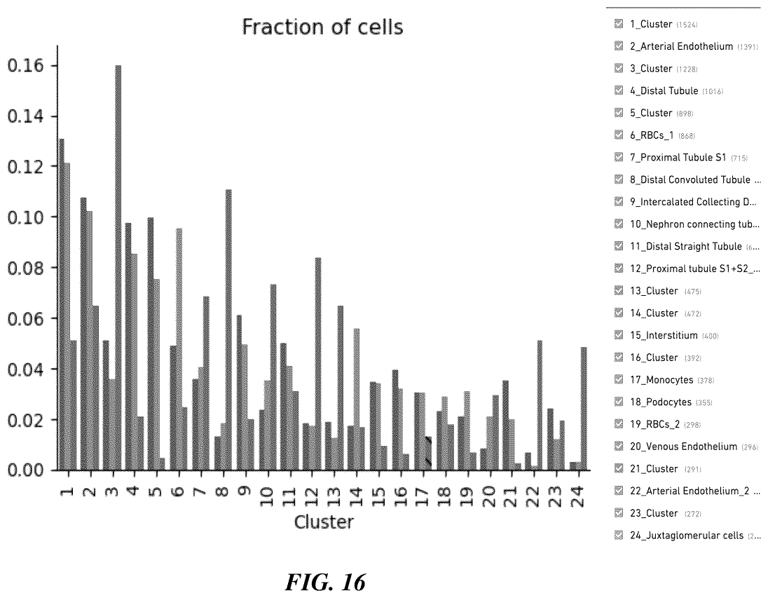

FIG. 18 includes another example of a transposase-nucleic acid complex for use in the methods provided herein. Transposase-nucleic acid complex 1800 (e.g., comprising a transpose dimer) comprises forked adapters 1801 and 1806, which forked adapters are partially double-stranded oligonucleotides. Partially double-stranded oligonucleotide 1801 comprises transposon end sequence 1803, first primer sequence 1802, second primer sequence 1805, and a sequence 1804 that is complementary to transposon end sequence 1803. Partially double-stranded oligonucleotide 1806 comprises transposon end sequence 1807, first primer sequence 1808, second primer sequence 1818, and a sequence 1809 that is complementary to transposon end sequence 1807. Primer sequences 1802, 1805, 1808, and 1810 may be the same or different. In some cases, primer sequences 1802 and 1808 may be designated as a first sequencing primer, such as an Illumina “R1” sequence and primer sequences 1805 and 1810 may be designated as a second sequencing primer, such as an Illumina “R2” sequence. Alternatively, primer sequences 1802 and 1810 may be designated “R1” and primer sequences 1805 and 1808 may be designated “R2”. Alternatively, primer sequences 1802 and 1808 may be designated “R2” and primer sequences 1805 and 1810 may be designated “R1”. Alternatively, primer sequences 1802 and 1810 may be designated “R2” and primer sequences 1805 and 1808 may be designated “R1”. Transposon end sequences 1803 and 1807 may be the same or different.

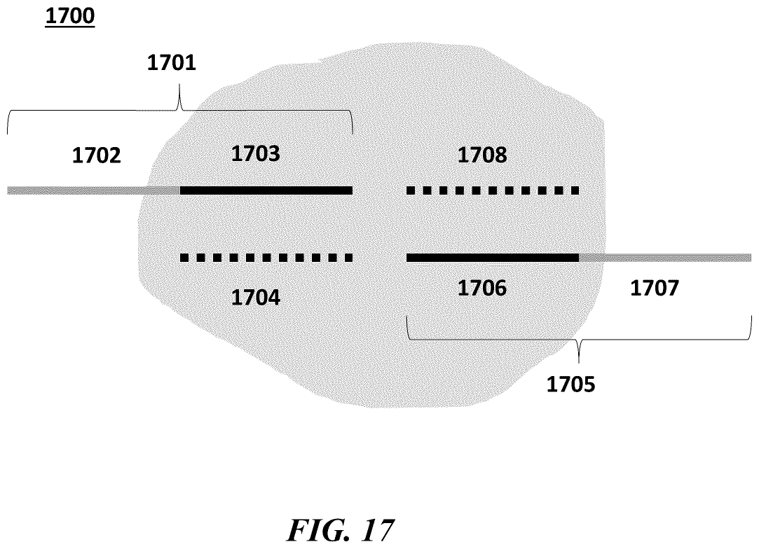

FIG. 19 shows transposase-nucleic acid complex 1900 (e.g., comprising a transpose dimer) comprising hairpin molecules 1901 and 1906. Hairpin molecule 1901 comprises transposon end sequence 1903, first hairpin sequence 1902, second hairpin sequence 1905, and a sequence 1904 that is complementary to transposon end sequence 1903. Hairpin molecule 1906 comprises transposon end sequence 1907, third hairpin sequence 1908, fourth hairpin sequence 1910, and a sequence 1909 that is complementary to transposon end sequence 1907. Hairpin sequences 1902, 1905, 1908, and 1910 may be the same or different. For example, hairpin sequence 1905 may be the same or different as hairpin sequence 1910, and/or hairpin sequence 1902 may be the same or different as hairpin sequence 1908. Hairpin sequences 1902 and 1908 may be spacer sequences or adapter sequences. Hairpin sequences 1905 and 1910 may be a promoter sequence such as T7 recognition or promoter sequences and/or UMI sequences. Transposon end sequences 1903 and 1907 may be the same or different. In some cases, sequence 1904 is a transposon end sequence and 1903 is a sequence complementary to sequence 1904. In some cases, sequence 1909 is a transposon end sequence and 1907 is a sequence complementary to sequence 1109.

Contacting a biological particle (e.g., cell or cell nucleus) comprising one or more target nucleic acid molecules (e.g., DNA molecules) with a transposase-nucleic acid complex may generate one or more tagged (see, e.g., FIGS. 17-19) template nucleic acid fragments (e.g., “tagmented fragments”). The one or more template nucleic acid fragments may each comprise a sequence of the one or more target nucleic acid molecules (e.g., a target sequence). The transposase-nucleic acid complex may be configured to target a specific region of the one or more target nucleic acid molecules (e.g., conjugated to an antibody specific for a protein bound to the target sequence) to provide one or more template nucleic acid fragments comprising specific target sequences. The one or more template nucleic acid fragments may comprise target sequences corresponding to accessible chromatin. A template nucleic acid fragment (e.g., tagmented fragment) may comprise one or more gaps (e.g., between a transposon end sequence or complement thereof and a target sequence on one or both strands of a double-stranded fragment). Gaps may be filled via a gap filling process using an enzyme, e.g., a polymerase (e.g., DNA polymerase). In some cases, a mixture of enzymes may be used to repair a partially double-stranded nucleic acid molecule and fill one or more gaps. Gap filling may not include strand displacement. Gaps may be filled within or outside of a partition.

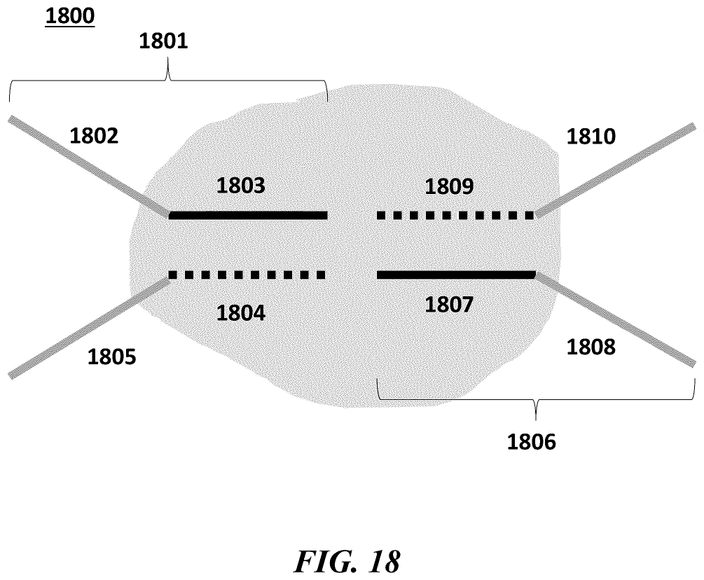

FIG. 20 includes a general workflow for methods provided herein. The left panel of the figure includes chromatin workflow 2000. Tagmentation may be performed in bulk to generate tagmented fragments of genomic DNA. The gDNA fragments may comprise one or more gaps (e.g., as described herein). A biological particle comprising one or more gDNA fragments may then be partitioned within a partition (e.g., droplet or well) with one or more reagents, including a first nucleic acid barcode molecule and a second nucleic acid barcode molecule (e.g., as described herein). One or more gDNA fragments of the partitioned biological particle and the first nucleic acid barcode molecule may be used to generate a first barcoded nucleic acid molecule. Gap filling may optionally be performed within the partition before or after generation of the first barcoded nucleic acid molecule. In some cases, gap filling may be performed prior to partitioning. In other cases, gap filling may be performed after recovery of the first barcoded nucleic acid molecule, or a derivative thereof, from the partition. Upon recovery from the partition, the first barcoded nucleic acid molecule, or a derivative thereof, may be subjected to additional processing to, e.g., incorporate additional functional groups including sequencing primers, flow cell adapters, identifying sequences, etc. and/or to amplify the barcoded nucleic acid molecule, or a derivative thereof, to enrich a sample population for subsequent analysis such as nucleic acid sequencing. The right panel of FIG. 20 includes RNA workflow 2050. Following partitioning, the RNA molecule (e.g., an mRNA molecule) and the second nucleic acid barcode molecule that may have the same or a different sequence as the first nucleic acid barcode molecule may be used to generate a second barcoded nucleic acid molecule. The second barcoded nucleic acid molecule, or a derivative thereof, may be recovered from the partition with the first barcoded nucleic acid molecule and optionally subjected to additional processing to, e.g., incorporate additional functional groups including sequencing primers, flow cell adapters, identifying sequences, etc. and/or to amplify the barcoded nucleic acid molecule, or a derivative thereof, to enrich a sample population for subsequent analysis such as nucleic acid sequencing. The first and second barcoded nucleic acid molecules, or derivatives thereof, may be separated from one another prior to undergoing such additional processing or may be processed in a common (e.g., bulk) solution. Similarly, the first and second barcoded nucleic acid molecules, or derivatives thereof, or amplicons thereof, may be subjected to nucleic acid sequencing at a same time and/or using a same system, or may be subjected to nucleic acid sequencing at a different time and/or using a different system.

In one aspect, the sample preparation methods, kits, compositions, and systems described herein may be used as part of a method of nucleic acid analysis, such as genomic DNA (gDNA) and optionally in tandem RNA, on a single cell/single nucleus basis. The analysis of gDNA and RNA (e.g., mRNA) may be from the same cell or same nucleus and optionally may be simultaneous.

In one embodiment, the method comprises the step of generating tissue fragments from a biological tissue sample. In another embodiment, the biological tissue sample is a solid tissue sample. In an additional embodiment, the tissue fragments are substantially free of dissociated cells. In one other embodiment, the method further comprises treating said tissue fragments with a fixing reagent to provide fixed tissue fragments. In other embodiments, the method further comprises dissociating said fixed tissue fragments to provide dissociated fixed tissue fragments which comprise fixed nuclei and/or fixed cells. In another embodiment, the dissociating step comprises treating said fixed tissue fragments with a cell dissociation reagent and/or an un-fixing agent. In one embodiment, the method further comprises generating a plurality of template nucleic acid fragments from and/or in said fixed nuclei or fixed cells using a plurality of transposase-nucleic acid complexes, each comprising a transposase molecule and a transposon end oligonucleotide molecule. In another embodiment, the method further comprises generating a plurality of partitions. In some embodiments, the plurality of partitions is a plurality of droplets in an emulsion or a plurality of wells. In one other embodiment, a partition of said plurality of partitions comprises (i) a single fixed nucleus or a single fixed cell, which comprises a template nucleic acid fragment of said plurality of template nucleic acid fragments, and (ii) a single support having attached thereto a plurality of barcode oligonucleotide molecules each comprising a barcode sequence. In some other embodiments, the support is a particle, such as a bead, e.g., a gel bead. In an additional embodiment, the method further comprises generating, in said partition, a barcoded nucleic acid fragment using at least (i) said template nucleic acid fragment, and (ii) a barcode oligonucleotide molecule of said plurality of barcode oligonucleotide molecules.

In one other aspect, the fixed nuclei or fixed cells from the dissociated fixed tissue fragments are subjected to an un-fixing step. In one embodiment, the un-fixing step comprises treating the fixed nuclei or fixed cells with a composition comprising an un-fixing agent, thereby providing un-fixed nuclei or un-fixed cells. In other embodiments, the composition comprises an un-fixing agent selected from compound (1), compound (2), compound (3), compound (4), compound (5), compound (6), compound (7), compound (8), compound (9), compound (10), compound (11), compound (12), compound (13), compound (14), compound (15), or a combination thereof. In another embodiment, the un-fixing agent comprises compound (8) or both compound (1) and compound (8). In another embodiment, the composition may further comprise a protease. In one embodiment, the un-fixing step is performed (to generate un-fixed nuclei or un-fixed cells) prior to the step of generating a plurality of template nucleic acid fragments through the use of a plurality of transposase-nucleic acid complexes, each comprising a transposase molecule and a transposon end oligonucleotide molecule. The un-fixing step allows for template nucleic acid fragment generation from and/or in the un-fixed nuclei or un-fixed cells.

### H. Chopping Devices

Freehand slicing or chopping of tissue with a hand-held razor blade or other cutting device is a common technique for preparing biological samples from tissue. Pre-scored molds have also been used to assist in chopping tissue and provide accuracy compared to freehand methods. The mold can allows the user to make consistent coronal or sagittal cuts with the aid of a razor blade. Sliding microtomes are also available but require training and practice and also require careful temperature control during use (see e.g., U.S. Pat. Nos. 7,827,894 and 1,865,539, each herein incorporated by reference in its entirety). Vibratomes and cryostat systems are also known but can be expensive and/or require training to use (e.g., see U.S. Pat. Nos. 7,237,392, and 4,752,347, and U.S. Pat. Publ. No. 20160084741, each herein incorporated by reference in its entirety).

Although the chop-fix methods as disclosed herein can be carried out by chopping tissue with a hand-held razor blade, the present disclosure also provides a mechanical chopping device that can be used to chop tissue in the chop-fix methods. Generally, such a chopping device comprises a plurality of aligned razor blades configured with a mechanism for reproducible hand-held chopping of a tissue sample in a dish and is assembled and disassembled by the user for facile cleaning.

Accordingly, in at least one embodiment the present disclosure provides a chopping device comprising:
(a) a razor blade holder, preferably a circular holder, comprising a plurality of razor blade slots, each slot configured to insert a cutting edge of a razor blade through the razor blade slot and hold a top portion of the razor blade within the razor blade slot, wherein a bottom portion of the razor blade including the razor blade cutting edge extends vertically below the razor blade holder;
(b) a razor blade alignment layer, preferably circular layer, comprising a plurality of alignment openings, wherein each alignment opening is configured to insert the cutting edge of the razor blade through the alignment layer so that the bottom portion of the razor blade including the razor blade cutting edge extends below the aligner layer; and
(c) a specimen dish holder configured to hold a specimen dish containing a biological tissue sample;
wherein the razor blade holder is configured to be placed on top of and connected to the razor blade alignment layer so that each of the plurality of razor blade slots are aligned above each of the corresponding plurality of alignment openings; and
wherein the razor blade alignment layer is configured to be placed on top of and reversibly and rotatably connected to the specimen dish holder so that each razor blade cutting edge extends into and toward the bottom of the specimen dish holder.

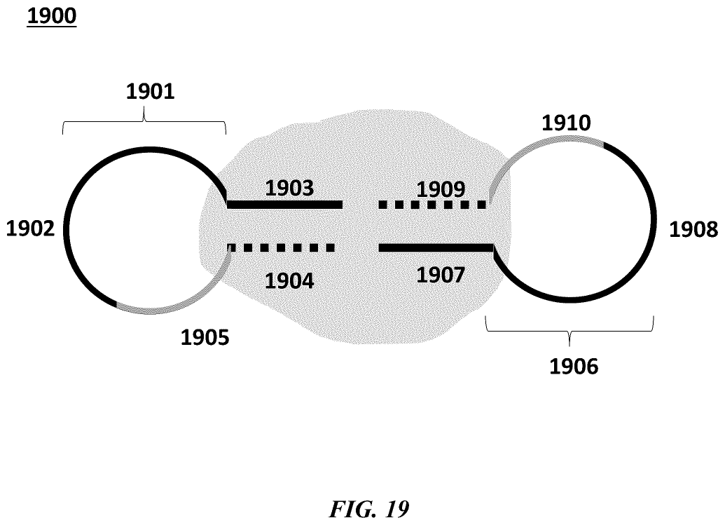

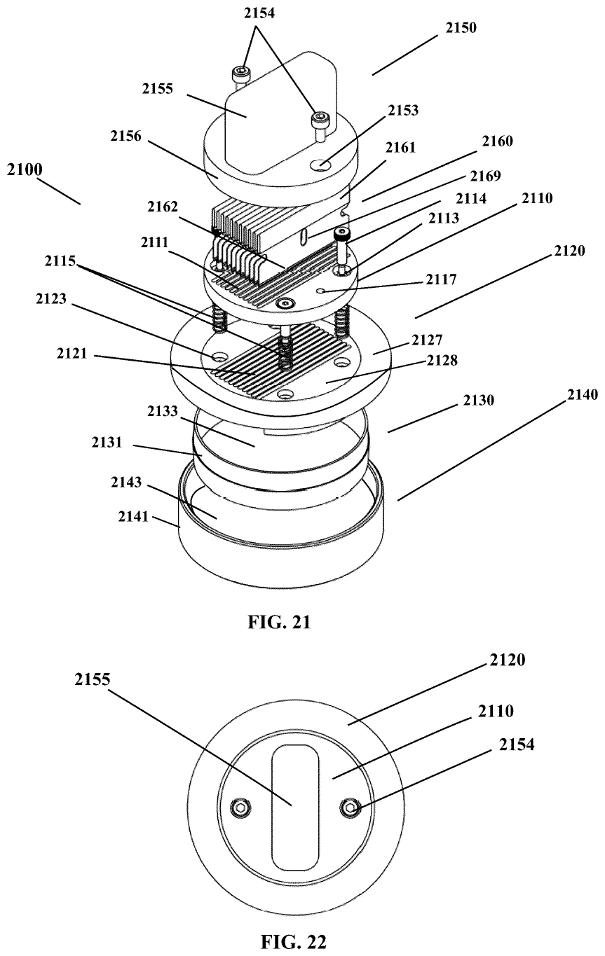

FIG. 21 provides an unassembled exploded depiction of such a chopping device 100 useful in the various chop-fix methods of the present disclosure. The chopping device 2100 includes razor blade holder 2110 aligned above razor blade alignment layer 2120, which in turn is aligned above specimen dish 2130 which in turn is aligned above specimen dish holder 2140. Each of the components 2110, 2120, 2130, and 2140 are configured to be stacked as indicated in FIG. 21 (and as shown assembled in FIG. 23A).

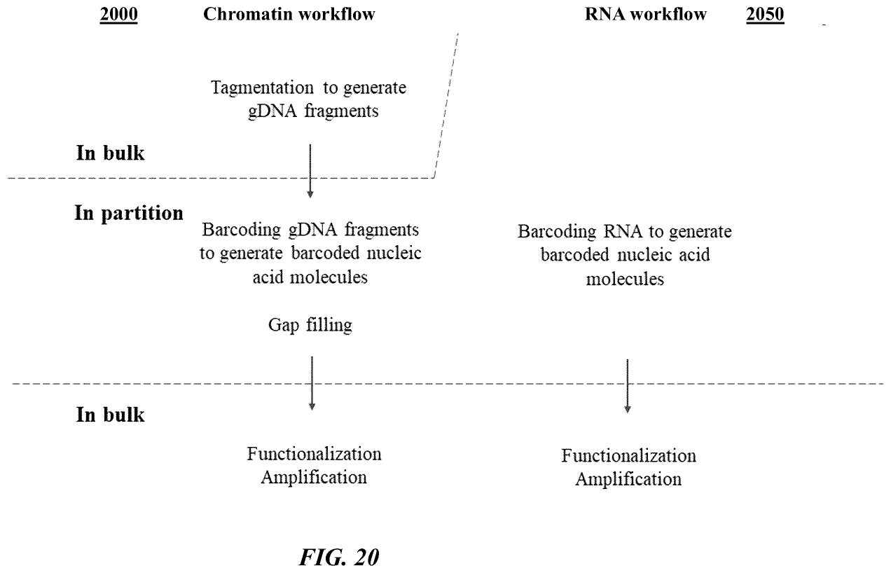

FIG. 22 shows a top view of the chopping device of FIG. 21 as assembled including razor blade alignment layer 2120 having diameter larger than razor blade holder 2110 and grip 2155 shown in center and a screw 2154 on each side of grip 2155.

Preferably, the razor blade holder 2110 and razor blade alignment layer 2120 are configured to rotate relative to specimen dish 2130 and/or dish holder 2140. Preferably, the razor blade holder, razor blade alignment layer, specimen dish holder and specimen dish are circular as shown in FIG. 21. In one embodiment, each of the components 2110, 2120, 2130, and 2140 are preferably circular as shown in FIG. 21 and, preferably are configured to fit together (and aligned when stacked, as shown in FIG. 23A).

The razor blade holder 2110 is preferably plate-like or disc-like as shown having a top surface and bottom surface and circular side circumference. Razor blade holder 2110 comprises a plurality of razor blade slots/openings 2111 as shown in FIG. 21, each slot/opening 2111 is configured for insertion of a razor blade edge as shown.

As shown in FIG. 21, the system preferably includes razor blades 160 aligned above the plurality of razor blade slots/openings 2111. Each elongated razor blade slots/openings 2111 is configured to allow the insertion of a single razor blade 2160. Preferably, each slot/opening has a width large enough for insertion of blade edge 162 but small enough to prevent the entire blade from passing through. For example, single edge razor blades 2160 having a backing 2161 are preferred for use and, if used, the slots/openings 2111 have a width larger than the thickness of the cutting edge 2162 (e.g., slot width of 0.010 inches vs edge thickness of 0.009 inches), but smaller than the thickness of the razor backing 2161 (e.g., backing thickness of 0.012 inches) allowing the edge of the blade to be inserted through the opening but the top of the razor blade held by the razor blade holder 2110.

Preferably, each slot/opening has a length ranging from 1 to 2 inches, more preferably 1.3 to 1.7 inches and even more preferably 1.4 to 1.6 inches.

Preferably, each slot/opening has a length less than 0.5 inches greater than length of blades being used, more preferably less than 0.2 inches greater than length of blades being used and even more preferably less than 0.1 inches greater than length of blades being used.

Preferably, each slot/opening has a width ranging from 0.005 to 0.02 inches, more preferably 0.006-0.014 inches and even more preferably 0.009 to 0.0012 inches.

Preferably, each slot/opening has a width less than 0.005 inches greater than the thickness of the cutting edge of the blades being used, more preferably less than 0.001 inches greater and even more preferably less than 0.0005 inches greater.

As shown in FIG. 21, the alignment layer 2120 comprises a plurality of alignment openings 2121 configured to aligned with the razor blade slots/openings 2111, each slot/opening 2111 is configured for insertion of a razor blade edge and to align and stabilize the razor blades vertically. According to preferred embodiments, the razor blade holder 2110 is circular and comprises at least two through-holes 2113 and at least two screws 2114 configured to be inserted through the at least two through-holes for reversibly connecting the circular razor blade holder 2110 onto the razor blade alignment layer 2120. The use of the term “circular” in “circular razor blade holder”, for example, refers to the holder component being “circular” versus defining the razor blades as circular. Preferably, each of the least two screws 2114 are a spring-loaded screw (e.g., shank and/or threading/length of screws 2114 surrounded by spring 115 as shown in FIG. 21) configured to provide a vertical lift force to the circular razor blade holder relative to the alignment layer to provide a collapsible gap (shown as gap 2319 in FIG. 23A) between the razor blade holder and alignment layer. Preferably, spring 2115 is a compression spring.

According to preferred embodiments, the circular razor blade holder comprises at least four through-holes and at least four screws inserted through the at least four through-holes for connecting the circular razor blade holder onto the razor blade alignment layer.

Preferably each of the least four screws is a spring-loaded screw configured to provide a vertical lift force to the circular razor blade holder relative to the alignment layer to provide a collapsible gap between the razor blade holder and alignment layer. The alignment layer 2120 preferably comprises one or more recesses 2123 configured for receipt of the ends of screws/springs 2114/2115, more preferably two or more recesses 2123, and even more preferably four or more recesses 2123. FIG. 23A is a side view of chopping device 2300 shown in resting position (or normal or non-compressed position). Chopping device 2300 comprises handle 2350, razor blade holder 2310, razor blades 2360, spring screws 2314/2315, alignment layer 2320 and specimen dish holder 2340, as shown. Handle 2350 comprises grip 2355 connected to base 2356, which is connected, preferably reversibly connected, to the top of razor blade holder 2310. Razor blade holder 2310 holds plurality of razor blades 2360 in razor blade holder openings (not shown). Alignment layer 2320 is shown fitted on top of specimen dish holder 2340 and aligns the plurality of razor blades 2360 vertically. According to alternative embodiments, the specimen dish holder 2340 is omitted and alignment layer 2320 is configured to be reversibly fitted on top of an open specimen dish (not shown in FIG. 23A).

Spring screws 2314/2315 connect razor blade holder 2310 on top of razor blade alignment layer 2320 wherein springs 2315 are configured and/or biased to hold razor blade holder 2310 above alignment layer 2320 resulting in compressible gap 2319 as shown in FIG. 23A. Preferably, springs 2315 are compression springs. To actuate the cutting or slicing action, handle 2350 is pressed down to push razor blades 2360 into the tissue sample held within the specimen dish (not shown). FIG. 23B shows razor blade holder 2310 pushed down onto alignment layer 2320 reducing or eliminating gap 2319 as shown.

FIG. 23C is an elevational side view of the chopping device of FIG. 23A in resting or normal position. FIG. 23D is an elevational side view of the chopping device of FIG. 23A in slicing or compressed position with grip 2355 indicating the orientation of the razor blades 2360 has been rotated relative to specimen dish holder 2340.

Preferably, the device is configured so that the cutting edges of the razor blades fully retract into the alignment openings at the resting or non-compressed position and material sticking to the cutting edge of a blade is pushed off the cutting edge upon retraction.

Preferably, the device is configured so that the cutting edges of the razor blades are retracted or lifted off the tissue or material in the specimen dish so that the razor blades can be rotated relative to the specimen dish without moving the tissue or material.

Preferably, the spring-loaded screws are configured so the gap between the bottom of the razor blade holder and the top of the alignment layer ranges from 0.1 to 0.5 inches, at rest or in non-compressed position (FIG. 23A), more preferably from 0.13 to 0.3 inches and most preferably from 0.15 to 0.2 inches.

According to alternative embodiments, the chopping device comprises at least two springs, preferably at least four springs configured to be placed between the circular razor blade holder relative and the alignment layer. According to one preferred embodiment, the chopping device includes springs, preferably compression springs, that surround each screw.

According to alternative embodiments, the chopping device comprises springs independent of the screws (not shown in FIG. 21) and, more preferably, the bottom surface of the razor blade holder and/or top surface of the alignment layer comprise recesses configured to receive an end of each spring to secure the location and/or orientation of the spring (not shown in FIG. 21). According to one preferred embodiment, the chopping device further comprises at least four springs configured to be placed between the circular razor blade holder and the alignment layer.

According to one preferred embodiment, the device comprises two or more screws, rivets, bolts or other means for connecting razor blade holder 2310 to razor blade alignment layer 2320 and two or more springs, preferably two or more compression springs, between razor blade holder 2310 and razor blade alignment layer 2320. Moreover, preferably the one or more springs surround the one or more screws, rivets, bolts or other means for connecting.

According to preferred embodiments, as shown in FIG. 21, the chopping device comprises a handle 2150 connected to the top of circular razor blade holder 2110. Preferably, the handle 2150 is connected to the circular razor blade holder with at least one screw 2154, more preferably with at least two screws 2154. Preferably, handle 2150 comprise one or more through-holes 2153 for insertion of the one or more screws 2154.

Preferably, handle 2150 includes grip 2155 configured to facilitate assembling and/or using chopping device 2100. For example, grip 2155 is configured to allow rotation of the handle 2150.

According to another preferred embodiment, the handle comprises a circular disk having at least one through-hole for a screw for connecting the handle to the circular razor blade holder. Preferably, the circular disk has a top side comprising a handle or grip component.

Preferably, handle 2150 comprises grip 2155 connected to handle base 2156. According to preferred embodiments, grip 2155 and handle base 2156 are integral or a single component. According to alternative embodiments, grip 2155 is attached to handle base 2156 (via glue, screws, interlocking mechanisms or other connecting means).

According to preferred embodiments, handle 2150 comprises one or more through-holes 153 for insertion of the one or more screws 2154 through handle base 2156.

According to preferred alternative embodiments, the circular razor blade holder comprises a top handle or grip, e.g., handle or grip integral with circular razor blade holder, but configured to allow razor blades 2160 to be inserted into razor blade slots/openings 2111 (not shown).

According to preferred alternative embodiments, the handle is configured to reversibly snap onto or screw onto the top of the circular razor blade holder.

Preferably, the handle is configured to hold the razor blades in the circular razor blade holder after the razor blades are inserted into the circular razor blade holder and the circular razor blade alignment layer (e.g., reversibly lock razor blades into slot openings).

According to one alternative embodiment, the device comprises a dome-shaped razor blade holder with slots/openings passing from the top of the dome through the bottom surface and the dome is configured to provide a handle for using the device and component for holding the razor blades. According to one embodiment, the razor blades are dropped into the slots/openings and reversibly locked into place (e.g., locking top inserts, locking mechanism within dome, rod inserted through opening in dome and through holes 2169 in blades to lock in place).

According to preferred embodiments, the circular razor blade alignment layer 2120 has an alignment layer diameter and the plurality alignment openings 2121 are parallel and have a length of at least 75% of the holder diameter.

According to preferred embodiments, the circular razor blade holder 2120 has a holder diameter and the plurality razor blade slots are parallel and have a length of at least 85% of the holder diameter.

Preferably, the circular razor blade holder 2120 has a holder diameter and the circular razor blade alignment layer 2120 has an alignment layer diameter and the alignment layer diameter is larger than the holder diameter.

According to preferred embodiments, the circular razor blade alignment layer 2120 comprises an annular raised surface 2127 around the outer perimeter of the top surface resulting a circular recessed center surface 2128 having a recessed center diameter surrounded by the annular raised surface, wherein the recessed center diameter is sized to fit the diameter of the circular razor blade holder 2110. Preferably, the annular raised surface is from 0.5 to 10 mm higher than the circular recessed center surface, more preferably 1-4 mm higher and most preferred approximately 2 mm higher.

The device is preferably configured for use with conventional specimen plates or dishes.

The device is preferably configured for use as a hand-held device for slicing or homogenizing tissue samples.

According to preferred embodiments, the device comprises at least one reusable specimen plate or dish 2130.

According to preferred embodiments, the device comprises circular specimen dish holder 2140 configured to hold at least one reusable specimen plate or dish 2130, as shown in FIG. 21.

Preferably, the circular specimen dish holder 2140 is a bowl configured to hold a specimen dish 2130.

Preferably, the circular specimen dish holder is a cylindrical dish configured to hold a cylindrical specimen dish.

According to preferred embodiments, the circular specimen dish holder has an external dish holder height, an internal dish holder height (e.g., dish holder outer wall 2141), an external dish holder diameter and an internal dish holder diameter. Preferably, the device further comprises a circular specimen dish 2130 having an external specimen dish height 2131, an internal specimen dish height, an external specimen dish diameter and an internal specimen dish diameter. Preferably, the internal dish holder diameter is greater than the external specimen dish diameter and the internal dish holder height is greater than the external specimen dish height.

Preferably, the circular specimen dish holder 2140 has a bottom surface 2143 configured to correspondence bottom surface 2133 of specimen dish 2130.

Preferably, the circular razor blade holder and the circular razor blade alignment layer are configured so that the cutting edge of each razor blade inserted into the slots and openings will contact the bottom of the specimen dish when the circular razor blade holder and the circular razor blade alignment layer are pushed down or in “slicing position” or “compressed position” (see FIG. 23B).

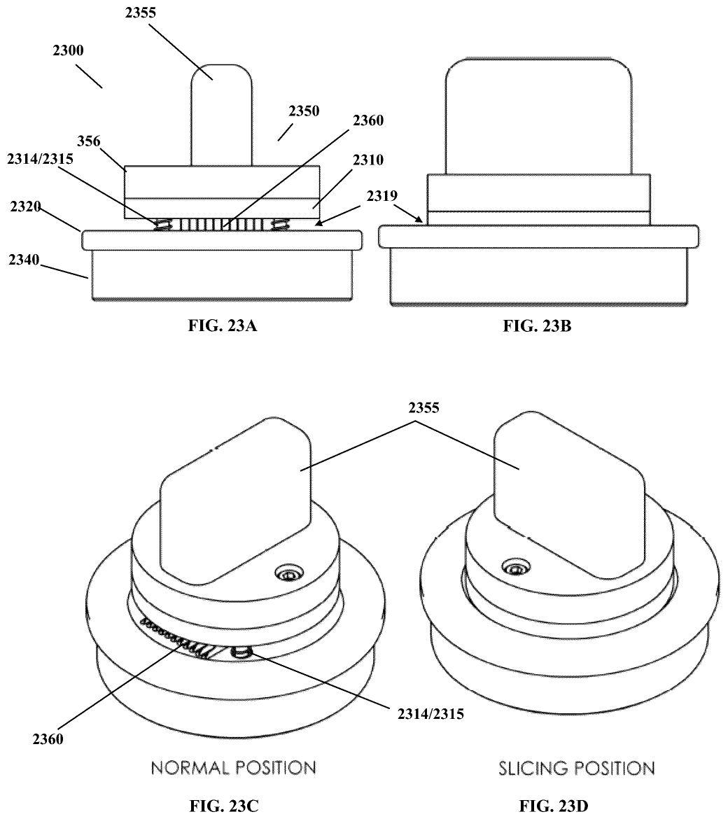

FIG. 24A is a side view illustration of slicing device 2400 in “resting position” as indicated by un-compressed gap 2419. Preferably, the chopping device when in resting or uncompressed position (e.g., FIG. 24A) has a height ranging from 2 to 4 inches, preferably 2 to 2.5 inches.

FIG. 24B is a cross-sectional side view illustration of the device of FIG. 24A in compressed or slicing position as shown by each cutting edge 2462 of razor blades 2460 contacting the interior bottom surface 2443 of specimen dish 2440 so that tissue (not shown) within the specimen dish 2440 would be sliced by the cutting edges of the blades.

According to preferred embodiments, the circular specimen dish holder 2440 comprises a top circular rim 2448 around the circumference of the holder and the alignment layer 2420 comprises an annular channel 2426 around the outer perimeter of the circular bottom surface and corresponding to the top circular rim 2448 of the specimen dish holder, wherein the annular channel 2426 is configured to receive the top circular rim 2448 to reversibly and rotatably connect the alignment layer 2420 onto the top of the circular specimen dish holder 2440.

Preferably, the alignment layer 2420 comprises a second annular channel 2427 around the outer perimeter of the circular bottom surface and within the inner circumference of the annular channel 2426, wherein the second annular channel 2427 is configured to receive a top circular rim of a specimen dish 2430 held within the circular specimen dish holder 2440.

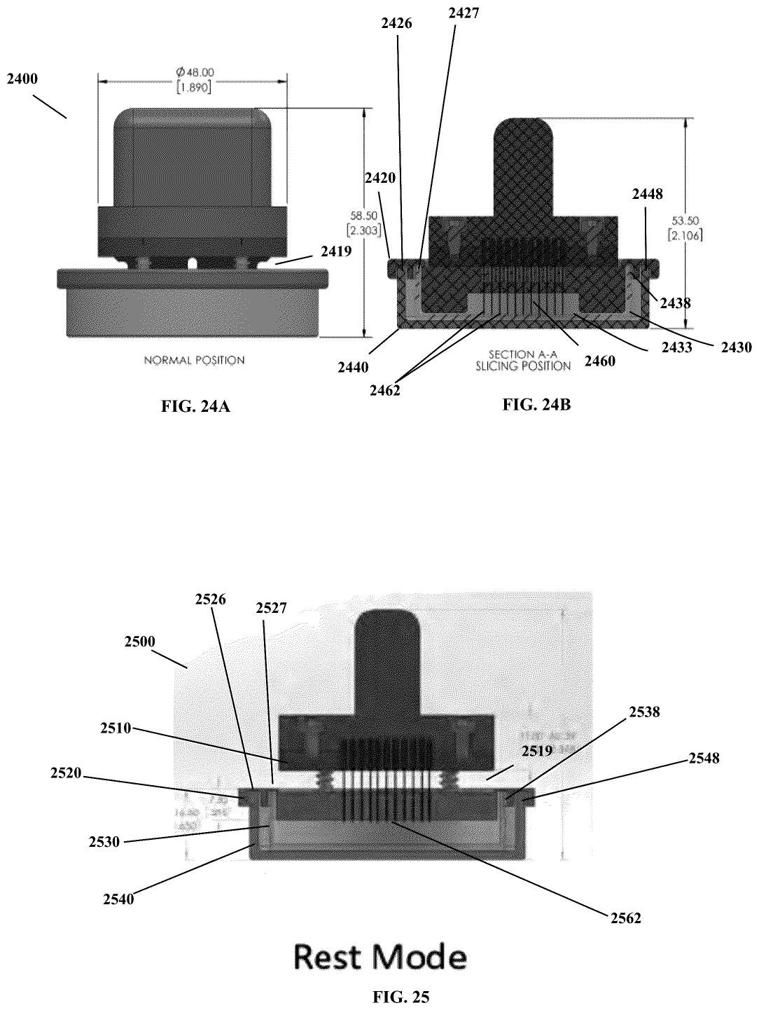

FIG. 25 is a cross-sectional side view of a chopping device 2500 according to another embodiment in “rest mode” or “normal” position as indicated by gap 2519. Razor blade cutting edges 2562 are shown raised above the bottom inner surface of specimen dish 2530 and nearly fully retracted into alignment layer 2520. Specimen dish holder 2540 includes a top rim 2548 and the bottom side of alignment layer 2520 includes an annular channel 2526 configured to receive top rim 2548 as shown. The bottom side of alignment layer 2520 preferably further includes a second annular channel 2527 within annular channel 2526 and configured to receive rim 2538 of specimen dish 2530 as shown. The annular channels 2526/2527 allow the alignment layer 2520 to be rotatably attached on top of specimen dish holder 2540 and specific dish 2530.

Preferably, the circular razor blade holder 2510 has a height ranging from 1 to 20 mm, more preferably from 2 to 10 mm, even more preferably from 4-6 mm and most preferred approximately 5 mm.

Preferably, the alignment layer 2520 has a thickness (or height) ranging from 5 to 30 mm, more preferably from 10 to 20 mm, even more preferably from 10-15 mm and most preferred approximately 13.5 mm.

Preferably, the circular specimen dish holder 2540 has an external height ranging from 5 to 30 mm, more preferably from 10 to 25 mm, even more preferably from 15-20 mm and most preferred approximately 16 mm.

Preferably, the razor blade holder 2510 is circular and has a diameter ranging from 1.5 to 2.5, more preferably 1.75 to 2.25 inches, even more preferably 1.85 to 2 inches.

Preferably, the alignment layer 2520 is circular and has an outer diameter ranging from 50 to 100 mm, more preferably from 60 to 90 mm, even more preferably from 65-75 mm and most preferred approximately 69 mm.

Preferably, the alignment layer 2520 has a circular inner diameter that sits within the circular specimen dish holder during use and ranges from 50 to 100 mm, more preferably from 60 to 90 mm, even more preferably from 65-75 mm and most preferred approximately 51.75 mm.

Preferably, the specimen dish holder 2540 is preferably circular and has an inner diameter ranging from 40 to 200 mm, more preferably from 40 to 100 mm, even more preferably from 55-75 mm and most preferred approximately 60 mm.

Preferably, the specimen dish holder 2540 is preferably circular and has an outer diameter ranging from 40 to 200 mm, more preferably from 40 to 100 mm, even more preferably from 55-75 mm and most preferred approximately 64 mm.

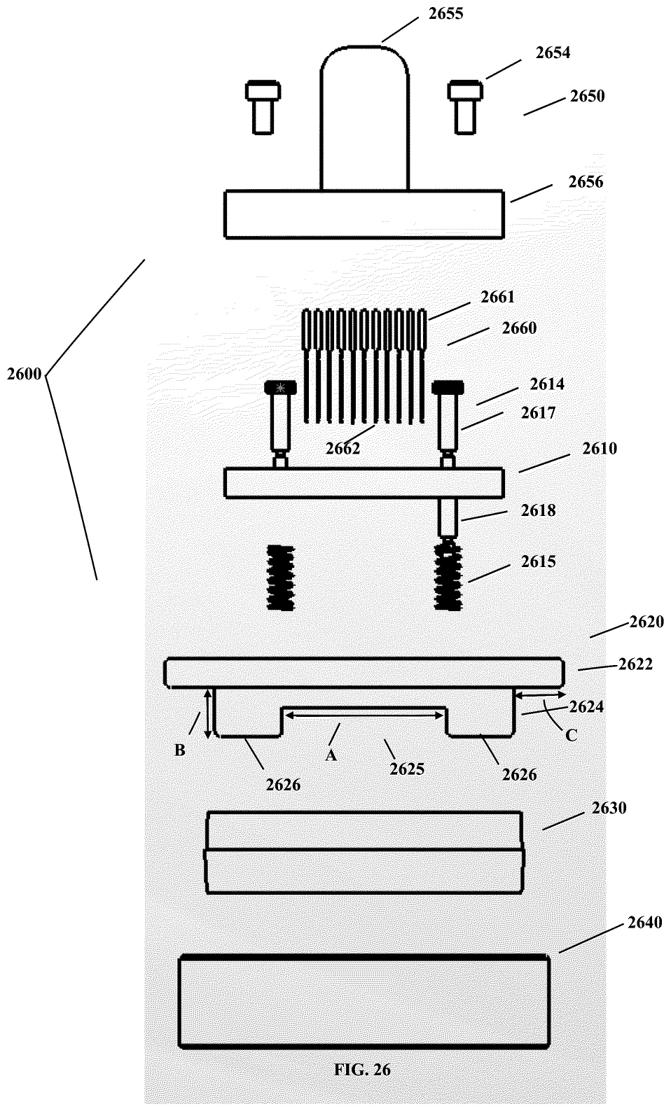

FIG. 26 shows a side view illustration of a chopping device 2600 according to another embodiment of the invention including razor blades 2660, specimen dish 630 and specimen dish holder 2640. Chopping device 2600 includes handle 2650, razor blade holder 2610, and alignment layer 2620. Handle 2650 comprises grip 2655 and base 2656 and is configured to be reversibly connected to the top of razor blade holder 2610 using screws or bolts 2654.

Razor blades 2660 are preferably eleven (11) single-edge blades each having cutting edge 2662 and backing 2661. Razor blade holder 2610 includes a plurality of razor blade slots (not shown) for insertion of cutting edges 2662 but configured to be narrower than backing 2661 to hold razor blades 2660 within razor blade holder 2610.

Razor blade holder 2610 is configured to be reversibly connected to the top of alignment layer 2620 using two or more screws 2614 (or bolts). Screws 2614 are preferably shoulder screws comprising a top shank portion 2617 configured to screw into a bottom shank portion 2618 and bottom shank portion 2618 is configured to fit within compression spring 2615, as shown. The shoulder screws are preferably used for screws 2654 and/or screws 2614 and/or other mechanical fasteners providing a bottom portion having different diameter from top portion and/or allowing the bottom portion to freely rotate to provide a freely rotating pin joint connection to another part (e.g., connecting razor blade holder to alignment layer).

Compression springs 2615 are configured to surround bottom portion 2618 of screw 2614 and configured to provide a compressible gap between razor blade holder 2610 and alignment layer 2620. Alignment layer 2620 preferably includes two or more recesses (not shown) on the top surface for receipt of screws 2614 and springs 2615. Alignment layer 2620 preferably includes a central recessed area on the top surface for receipt of the razor blade portion 2618 (as discussed above and below).

According to preferred embodiments, alignment layer 2620 has a top portion 2622 and a bottom portion 2624 (or structure connected to the bottom of the top portion 2622) configured to provide an opening 2625 for the cutting edges 2662 of razor blades 2660 and also provide one or more retaining members 2626 configured to retain tissue specimen (not shown) in the bottom of specimen dish 2630 beneath the cutting edges 2662 of razor blades 2660 and prevent tissue specimen (and/or reduce the tissue) from being pushed towards the sides of the bottom of the specimen dish 2630.

Preferably, opening A (between the retaining members 2626) is configured and sized for accommodating the cutting edges of the plurality of blades (e.g., allowing the cutting edges to pass down into the volume of the specimen dish to contact tissue contained therein). That is, the cutting edges are allowed to pass through the opening A and contact the bottom of the specimen dish while enclosed by retaining members 2626.

Preferably, the height B of retaining members 2626 is configured so the bottom surface of retaining members 2626 contacts the bottom surface of specific dish 2630 when the alignment layer 2620 is attached onto the specimen dish 2630 (e.g., the chopping device 2600 fully assembly for use) and the chopping device is compressed or in slicing position.

Preferably, retaining members 2626 are recessed from the outer circumference of the top portion 2622 creating a bottom recessed area C configured to accommodate a first annular channel (not shown) for the top rim of the specimen dish 2630 and a second annular channel (not shown) for the top rim of the specimen dish holder 2640.

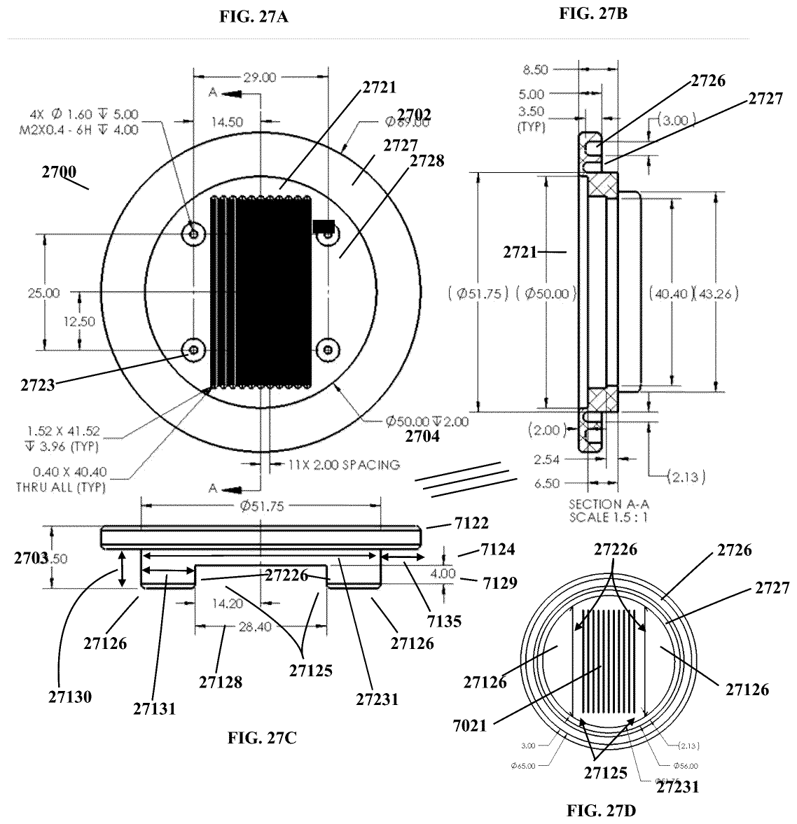

FIGS. 27A-27D are schematic drawings of alignment layer 2700 according to another preferred embodiment of the present disclosure. Alignment layer 2700 is preferably circular and preferably has an outer diameter 2702 ranging from 65-75 mm, more preferably 68-70 mm and most preferred approximately 69 mm and a total fixed height preferably ranging from 10-15 mm, more preferably 12-14 mm and most preferred approximately 13.5 mm.

FIG. 27A is a top view of circular alignment layer 2700 including an annular raised surface 2727 on the top surface around the outer circumference and surrounding circular recessed center surface 2728 having a recessed center diameter 2704, wherein the recessed center diameter is sized to fit the diameter of a circular razor blade holder (not shown). Preferably, the recessed center diameter 2704 ranges from 40-60 mm, more preferably from 45-55, even more preferably from 48-52 mm and most preferred approximately 50 mm.

Preferably, the annular raised surface 2727 is from 0.5 to 10 mm higher than the circular recessed center surface 2728, more preferably 1-2 mm higher and most preferred approximately 1.5 mm higher.

As shown in FIG. 27C, alignment layer 2700 has a top portion 27122 and a bottom portion 27124 (or structure connected to the bottom of the top portion 27122), wherein bottom portion 27124 is configured to provide an opening 27125 for accommodating the cutting edges of the razor blades and bottom portion 27124 also providing one or more retaining members 27126 configured to retain the tissue specimen (not shown) centrally in the bottom of the specimen dish beneath the razor blades and prevent tissue specimen (and/or reduce tissue) from being pushed towards the sides of the bottom of the specimen dish during the slicing action. Preferably, each retaining member 27126 has a retaining wall 27226 configured to, with the opposing retaining wall 27226, retain tissue between the retaining member 27126 during the slicing action of the razor blades.

Preferably, opening 27125 has a width 27128 (between walls 27227 of retaining members 27126) configured and sized for accommodating the cutting edges of the plurality of blades. That is, the cutting edges are allowed to pass through the opening A and contact the bottom of the specimen dish while enclosed by retaining members 27126. Preferably, width 27128 ranges from 20-40 mm, more preferably 25-35 mm, even more preferably 28-29 mm and most preferred approximately 28.5 mm.

Preferably, retainer member height 27130 is configured so the bottom surface of retaining members 27126 contact the bottom surface of the specific dish when the alignment layer 2700 is attached onto the specimen dish (e.g., the chopping device fully assembly for use) and the chopping device is compressed or in slicing position. Preferably, height 27130 ranges from 4-20 mm, more preferably from 6-12 mm, more preferred from 8-9 mm, and most preferred approximately 8.5 mm.

Preferably, retaining members 27126 are recessed from the outer circumference of the top portion 27122 creating a bottom recessed area 27135 configured to accommodate a first channel 2727 (shown in FIG. 27B) for the top rim of the specimen dish (not shown) and a second annular channel 2726 (shown in FIG. 27B) for the top rim of the specimen dish holder (not shown). Preferably, the width of recessed area 27135 ranges from 15-20 mm, more preferably from 17-18 mm and most preferred approximately 17.25 mm.

Preferably, the width of first annular channel 2727 ranges from 2 to 2.5 mm, more preferably from 2.1 to 2.2 mm and most preferred approximately 2.13 mm. Preferably, the width of second annular channel 2728 ranges from 2 to 4 mm, more preferably from 2.5 to 3.5 mm and most preferred approximately 3 mm.

FIG. 27D is a bottom view of alignment layer 2700 showing first annular channel 2727 and second annular channel 2728 and retaining members 27126 and opening 27125 having plurality of razor slots 2721 aligned within the opening 27125 between retaining walls 27226 as shown.

FIGS. 27A-27D show various dimensions of alignment layer component 2700 illustrating one preferred embodiment of the invention. Other preferred embodiments may include one or more different dimensions having substantially the same approximate dimensions.

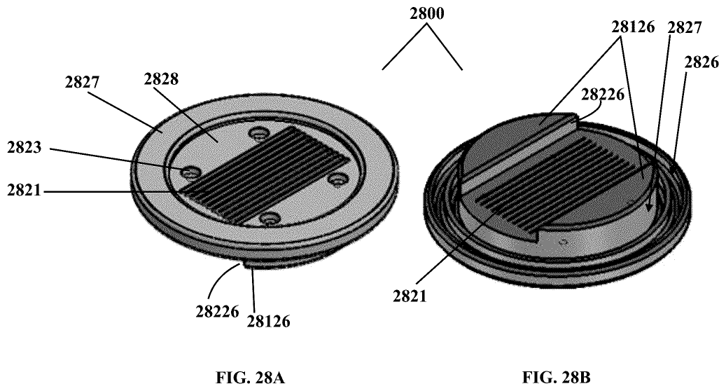

FIGS. 28A-28B are illustrations of alignment layer 2800 according to another preferred embodiment. FIG. 28A is a top perspective view of alignment layer 2800 having a top surface including a recessed area 2828 including a plurality of aligned razor slots 2821 and four screw holes 2823 configured to connect to circular razor blade holder with screws (not shown). Recessed area 2828 is shown within raised annular area 2827 and is configured to receive the bottom of the circular razor blade holder (not shown).

FIG. 28B is a bottom perspective view of alignment layer 2800 showing razor slots 2821 between retaining members 28126 having walls 28226. FIG. 28B also shows first annular channel 2827 within second annular channel 2826. FIG. 28B shows retaining walls 28226 of retaining members 28126 configured to enclose tissue specimen between the retaining walls 28226 of both retaining member 28126 when the device is compressed to maintain the tissue beneath the razor blades.

The chopping device is preferably configured to hold at least one razor blade configured to slice or homogenize a tissue or biological sample or specimen, preferably a plurality of razor blades, more preferably at least two razor blades, even more preferably at least five razor blades, even more preferred at least ten razor blades and most preferred eleven razor blades. According to one preferred embodiment the device is configured to hold or comprises at least five razor blade slots and at least five alignment openings, more preferably, at least ten razor blade slots and at least ten alignment openings, and preferably less than twenty (20) blades. According to one preferred embodiments, the razor blade holder comprises at least eleven razor blade slot openings and razor blade alignment layer comprises at least eleven alignment openings.

According to preferred embodiments, the razor blade slot opening comprises a top slot opening and a bottom slot opening, wherein the top slot opening is configured to hold the top of the razor blade (e.g., the thicker backing of a single edge razor blade) while the bottom slot opening is configured to allow the sharp cutting edge of the blades to pass through. Preferably, each razor blade slot opening has a length ranging from 35-45 mm and width ranging from 0.1-0.4 mm. Preferably, the top slot opening has dimensions ranging from 0.5-3 mm×30-50 mm, more preferably 1.0-2 mm×35-45 mm, and most preferred approximately 1.3 mm×40.64 mm. Preferably, the bottom slot opening has dimensions ranging from 0.1-1.5 mm×30-50 mm, more preferably 0.3-0.8 mm×35-45 mm and most preferred approximately 0.4 mm×40.4 mm.

According to preferred embodiments, the chopping device or kit comprises or the device is configured to hold a plurality of razor blades, wherein each razor blade has a cutting edge configured to fit through each razor blade slot and each alignment opening. Preferably, each alignment opening has a length ranging 30-50 mm, more preferably from 35-45 mm, and most preferred approximately 40.4 mm and a width ranging from 0.1-1.0 mm, more preferably 0.3-0.5 mm and most preferred approximately 0.4 mm.

Preferably, each razor blade is a single edge razor blade, preferably comprising a top backing (shown as backing 2161 in FIG. 21).

Preferably, each razor blade has a length ranging from 35-45 mm and a thickness ranging from 0.1-0.4 mm. According to preferred embodiments, each razor blade has a length of approximately 1.5 inches, a width of approximately 11/16 inch and a thickness of approximately 0.009 inch.

According to another embodiment, the chopping device or chopping device kit comprises a specimen dish, preferably a circular specimen dish. Preferably, the specimen dish has an outer diameter ranging from 40 to 65 mm, more preferably 50-60 mm and most preferred 54-55.25 mm, external dish height ranging from 10-20 mm, more preferably 12-15 mm and most preferred approximately 13.93 mm, internal dish height ranging from 7 to 20 mm, more preferably from 10-15 mm and most preferred approximately 12.43 mm and an inner diameter ranging from 39 to 59 mm, more preferably from 44-55 mm and most preferred approximately 52.75 mm.

According to alternative embodiments, the chopping device is used without a specimen dish holder and the alignment layer is rotatably attached directly onto the specimen dish.

In another aspect the present disclosure provides tissue preparation kits comprising, in one or more containers, at least one chopping device and/or one or more chopping device components configured for assembly (e.g., razor blade holder, razor blade alignment layer and specimen dish holder). In at least one embodiment of the invention relates to a kit comprising a chopping device as described herein and at least one blade, preferably at least one razor blade, more preferably includes a plurality of razor blades.

According to preferred embodiments, the kit further comprises a plurality of razor blades, preferably at least ten blades. According to preferred embodiments, the kit further comprises at least one specimen dish or plate. According to preferred embodiments, the kit further comprises at least one specimen dish holder. According to preferred embodiments, the kit further comprises two or more screws and/or springs, preferably compression springs. According to another preferably, the kit comprises two or more alignment layers having different dimensions (e.g., diameter, annular channel width) and/or two or more specimen dish holders having different dimensions (e.g., diameter) to accommodate specimen dishes having different sizes and designs.

In another aspect, the present disclosure also provides methods of using the chopping devices as described herein. In at least one embodiment of a method of using a chopping device of the present disclosure, the method comprises pressing the razor blade holder down thereby forcing the cutting edges of the plurality of razor blades onto and cutting the tissue sample.

In at least one embodiment of a method of using a chopping device of the present disclosure, wherein the chopping device comprises a plurality of razor blades inserted into the plurality of razor blade slots and through the plurality of alignment openings and placed on top of and reversibly and rotatably connected to the circular specimen dish holder containing an open circular specimen dish containing a tissue sample, the method comprises:
(i) pressing the razor blade holder down thereby forcing the cutting edges of the plurality of razor blades onto and cutting the tissue sample;
(ii) rotating the plurality of razor blades relative to the tissue sample to a second orientation; and
(iii) pressing the razor blade holder down thereby forcing the cutting edges of the plurality of razor blades onto the and cutting the tissue sample at the second orientation.

In at least one embodiment of the method, the chopping device is configured so that each pressing of the plurality of razor blades results in the orientation of the plurality of razor blades relative to the specimen dish being shifted by at least 10 degrees (e.g., external threading within specimen dish holder and alignment layer and springs result in automatic re-orientation after each pressing action).

In at least one embodiment, the method of using the chopping device further comprises rotating the plurality of razor blades relative to the tissue sample to a third orientation and pressing the razor blade holder down on the circular specimen dish holder thereby forcing the cutting edges of the plurality of razor blades onto the tissue sample and cutting tissue sample at the second orientation. Preferably, the plurality of razor blades is rotated at least 10-25 degrees, preferably 15-20 degrees, with each rotation.

In at least one embodiment of the method of using the chopping device, the method further comprises cleaning the chopping device before using to chop or homogenize another sample, preferably the method further comprises dis-assembling the chopping device and cleaning the dis-assembled components and re-assembly the components before next use. In at least one embodiment, the method further comprises replacing the blades before using to chop or homogenize another sample.

Additional embodiments of the present disclosure are provided in the numbered clauses below.

1. A method for nucleic acid analysis comprising
a) generating tissue fragments from a biological tissue sample;
b) treating said tissue fragments with a fixing reagent to provide fixed tissue fragments;
c) dissociating said fixed tissue fragments to provide dissociated fixed tissue fragments which comprise fixed nuclei; and
d) generating a plurality of template nucleic acid fragments in said fixed nuclei using a plurality of transposase-nucleic acid complexes, each comprising a transposase molecule and a transposon end oligonucleotide molecule.

2. The method of clause 1, further comprising e) generating a plurality of partitions, wherein a partition of said plurality of partitions comprises (i) a single fixed nucleus, which comprises a template nucleic acid fragment of said plurality of template nucleic acid fragments, and (ii) a plurality of barcode oligonucleotide molecules each comprising a barcode sequence.

3. The method of clause 2, further comprising f) in said partition, generating a barcoded nucleic acid fragment using at least (i) said template nucleic acid fragment, and (ii) a barcode oligonucleotide molecule of said plurality of barcode oligonucleotide molecules.

4. A method for nucleic acid analysis comprising
a) generating tissue fragments from a biological tissue sample;
b) treating said tissue fragments with a fixing reagent to provide fixed tissue fragments;
c) dissociating said fixed tissue fragments to provide dissociated fixed tissue fragments which comprise fixed nuclei; and
d) contacting an open chromatin structure of a fixed nucleus with a transposase-nucleic acid complex to yield said fixed nucleus comprising a tagmented fragment of a deoxyribonucleic acid (DNA) molecule, wherein said fixed nucleus further comprises a ribonucleic acid (RNA).

5. The method of clause 4, further comprising e) generating a plurality of partitions, wherein a partition of said plurality of partitions comprises (i) said fixed nucleus, (ii) a first nucleic acid barcode molecule comprising a first barcode sequence, (iii) a second nucleic acid barcode molecule comprising a second barcode sequence, and (iv) a splint molecule, wherein said first nucleic acid barcode molecule comprises an overhang sequence, and wherein said splint molecule comprises a first sequence complementary to a sequence of said tagmented fragment and a second sequence complementary to said overhang sequence.

6. The method of clause 5, further comprising, within said partition: (i) using said tagmented fragment, said first nucleic acid barcode molecule, and said splint molecule to generate a first barcoded nucleic acid product comprising said first barcode sequence or a reverse complement thereof and a sequence of said DNA molecule, and (ii) using said RNA molecule and said second nucleic acid barcode molecule to generate a second barcoded nucleic acid product comprising said second barcode sequence or a reverse complement thereof and a complementary DNA (cDNA) sequence of said RNA molecule.

7. A method for nucleic acid analysis comprising
a) generating tissue fragments from a biological tissue sample;
b) treating said tissue fragments with a fixing reagent to provide fixed tissue fragments;
c) dissociating said fixed tissue fragments to provide dissociated fixed tissue fragments which comprise fixed nuclei;
d) un-fixing said fixed nuclei with an un-fixing reagent; and
e) generating a plurality of template nucleic acid fragments in said un-fixed nuclei using a plurality of transposase-nucleic acid complexes, each comprising a transposase molecule and a transposon end oligonucleotide molecule.

8. The method of clause 7, further comprising e) generating a plurality of partitions, wherein a partition of said plurality of partitions comprises (i) a single un-fixed nucleus, which comprises a template nucleic acid fragment of said plurality of template nucleic acid fragments, and (ii) a plurality of barcode oligonucleotide molecules each comprising a barcode sequence.

9. The method of clause 8, further comprising f) in said partition, generating a barcoded nucleic acid fragment using at least (i) said template nucleic acid fragment, and (ii) a barcode oligonucleotide molecule of said plurality of barcode oligonucleotide molecules.

10. A method for nucleic acid analysis comprising
a) generating tissue fragments from a biological tissue sample;
b) treating said tissue fragments with a fixing reagent to provide fixed tissue fragments;
c) dissociating said fixed tissue fragments to provide dissociated fixed tissue fragments which comprise fixed nuclei;
d) un-fixing said fixed nuclei with an un-fixing reagent; and
e) contacting an open chromatin structure of an un-fixed nucleus with a transposase-nucleic acid complex to yield said un-fixed nucleus comprising a tagmented fragment of a deoxyribonucleic acid (DNA) molecule, wherein said un-fixed nucleus further comprises a ribonucleic acid (RNA).

11. The method of clause 10, further comprising e) generating a plurality of partitions, wherein a partition of said plurality of partitions comprises (i) said un-fixed nucleus, (ii) a first nucleic acid barcode molecule comprising a first barcode sequence, (iii) a second nucleic acid barcode molecule comprising a second barcode sequence, and (iv) a splint molecule, wherein said first nucleic acid barcode molecule comprises an overhang sequence, and wherein said splint molecule comprises a first sequence complementary to a sequence of said tagmented fragment and a second sequence complementary to said overhang sequence.

12. The method of clause 11, further comprising, within said partition: (i) using said tagmented fragment, said first nucleic acid barcode molecule, and said splint molecule to generate a first barcoded nucleic acid product comprising said first barcode sequence or a reverse complement thereof and a sequence of said DNA molecule, and (ii) using said RNA molecule and said second nucleic acid barcode molecule to generate a second barcoded nucleic acid product comprising said second barcode sequence or a reverse complement thereof and a complementary DNA (cDNA) sequence of said RNA molecule.

13. The method of any one of the preceding clauses 1-12, wherein said dissociating step comprises treating said fixed tissue fragments with a cell dissociation reagent.

14. The method of any one of the preceding clauses 1-13, wherein said tissue fragments from step a) are substantially free of dissociated cells.

15. The method of any one of the preceding clauses 1-14, wherein the biological tissue sample is a solid tissue sample.

16. The method of any one of the preceding clauses 1-15, wherein the plurality of barcode oligonucleotide molecules is attached to a support.

17. The method of clause 16, wherein the support is a bead.

18. The method of clause 17, wherein the bead is a gel bead.

19. The method of clause 16, wherein the plurality of barcode oligonucleotide molecules is releasably attached to said support.

20. The method of any one of the preceding clauses 1-19, further comprising lysing of permeabilizing said fixed or un-fixed nucleus to provide access to said tagmented fragment.

21. The method of any one of the preceding clauses 1-20, wherein said transposase-nucleic acid complex comprises a first adapter and a second adapter and wherein said tagmented fragment comprises a sequence of said DNA molecule flanked by said first adapter and said second adapter.

22. The method of clause 12, wherein (i) comprises hybridizing said splint molecule to A) said first or said second adapter of said tagmented fragment and B) said first nucleic acid barcode molecule and ligating said first nucleic acid barcode molecule and said tagmented fragment to generate said first barcoded nucleic acid product.

23. The method of clause 22, wherein said first adapter comprises a first transposon end sequence and a first primer sequence and wherein said second adapter comprises a second transposon end sequence and a second primer sequence.

24. The method of clause 23, wherein (i) comprises A) hybridizing said splint molecule to 1) said first primer sequence or said second primer sequence of said tagmented fragment and 2) said first nucleic acid barcode molecule and B) ligating said first nucleic acid barcode molecule and said tagmented fragment to generate said first barcoded nucleic acid product.

25. The method of clause 22, wherein said first primer sequence or said second primer sequence is single stranded.

Additional methods, compositions, systems, and kits for processing DNA and/or RNA nucleic acid molecules from a cell or cell nucleus are disclosed in U.S. Pat. Pub. No. 20200291454A1, which is herein incorporated by reference in its entirety.

### EXAMPLES

Various features and embodiments of the disclosure are illustrated in the following representative examples, which are intended to be illustrative, and not limiting. Those skilled in the art will readily appreciate that the specific examples are only illustrative of the embodiments of the disclosure as described more fully in the claims which follow thereafter. Every embodiment and feature described in the application should be understood to be interchangeable and combinable with every embodiment contained within.

### Example 1: Chop-Fixed Preparation of Dissociated Fixed Cells from Mouse Brain Tissue

This example illustrates the use of the chop-fixed method preparation of dissociated PFA-fixed cells from mouse brain tissue. The biological sample of dissociated PFA-fixed cells prepared by this method can be used in a range of partition-based single cell assays including RNA-templated ligation or reverse-transcription of nucleic acids, as described in the further Examples below.

### Materials and Methods

### A. Tissue Chopping

A sample of fresh mouse brain tissue was placed in a clean petri dish on ice until ready to chop. A single clean razor blade was used to finely chop the brain tissue by hand with as small a step size as possible between each cut. Care was taken to avoid dragging or swirling the tissue during chopping. This chopping process was repeated across the entire brain tissue on 3 different axes to maximize fine chopping of the sample. The resulting chopped mouse brain tissue consisted of chopped particles of tissue having average dimensions of 0.5 mm×0.5 mm.

### B. Fixation of Chopped Tissue Particles

The finely chopped particles of mouse brain tissue were immediately immersed in a 4% PFA solution. Control samples were also prepared with chopped tissue but without fixation by immersing the chopped tissue particles directly in a solution of dissociation reagent. The tube, with a ratio of 10× the volume of 4% PFA solution to the volume of tissue, was stored on its side at 4C overnight. After the overnight storage, the samples were spun down at 500 rcf for 5 minutes. The fixative solution supernatant was removed. The fixed tissue at the bottom of the tubes was resuspended with a cell resuspension buffer (1% BSA+0.2 U/mL RNase inhibitor in PBS).

### C. Dissociation of Fixed Tissue Particles

The resuspended sample was transferred with a wide bore P1000 pipette to a new tube and spun down again (at 500 rcf) for 2 minutes. Most resuspension buffer was removed and the remaining ˜1 mL that was a slurry of fixed brain tissue (250 mg of tissue) was transferred to a 4 mL tube containing 1 mL of the dissociation reagent, Collagenase A (Roche KGaA, Darmstadt, DE; Millipore-Sigma cat. #11088793001) at 2.5 mg/mL in PBS. The fixed tissue in dissociation reagent mixture was shaken at 700 rpm for ˜90 min at 37 C.

While still in the tube, the fixed tissue dissociation reagent mixture was pipetted with a wide bore P1000 20 times, and then with a normal bore P1000 20 times. After pipetting the tube was again spun down, it was washed and resuspended twice with 1% BSA in PBS+RNase inhibitor (Roche KGaA, Darmstadt, DE; Millipore-Sigma cat #333539900), then filtered through a 40 μm pore size Flowmi or Pluriselect filter on top of 50 mL falcon tube, using a large plunger to pull sample through. The filtered sample was further spun down at 500 rcf for 5 minutes and resuspended in buffer to provide final dissociated fixed cell sample.

Results: The dissociated fixed cells in the final sample prepared from the chop-fixed brain tissue can be visualized and counted using SYTO RNA dye (1:1000) and/or ethidium homodimer (1:1 after resuspending stock with 200 μL of water per aliquot) both kept at −20C. Trypan Blue can be used to visualize any remaining debris more easily.

### Example 2: Single-Cell 3′ Analysis of Chop-Fixed Mouse Kidney and Brain Tissue

This example illustrates a comparative study using the chop-fixed method to prepare mouse kidney and brain tissue samples for single-cell 3′ sequence analysis carried up to 7 days post-fixation.

### Materials and Methods

### A. Sample Preparation:

Chop-fixed cell samples were prepared from mouse kidney tissue and mouse brain tissue as described in Example 1.

Additionally, the following control samples were prepared: (1) suspended PBMCs (not from tissue) fixed with 4% PFA; (2) kidney tissue fixed cells prepared without chopping but simply by treatment of the tissue with dissociating reagent followed by fixing in 4% PFA; (3) fresh control PBMCs were thawed from cryopreservation with RPMI and 10% FBS; (4) fresh control kidney tissue sample (without fixation) was prepared by chopping the fresh kidney tissue followed by incubation with a mixture of 2.5 mg/ml Collagenase A (Roche) and 10 mg/mL Pancreatin (Sigma, P1625) at 37 C for 30 minutes; and (5) fresh control brain tissue sample (without fixation) was prepared by chopping fresh brain tissue and incubating with 2 mg/mL papain in Hibernate® E-Ca (HE-Ca) Hibernate® A-Ca (HA-Ca) without B27™ for adult dissociations (Brainbits) for 20 minutes at 30 C, then washed twice with Hibernate twice and resuspended with Nbactiv1 (Brainbits).

### B. Single-Cell 3′ Sequence Analysis

Partitioning of Un-Fixed into GEMs and 3′-RT

Pellet fractions collected from the un-fixing/protease treatment were centrifuged at 5 min 300 g and washed with PBS 0.04% BSA twice before loaded into the Single Cell 3′V3 protocol standard master mix used with the Chromium System (10× Genomics, Pleasanton, CA, USA) for partitioning samples together with barcoded gel beads in discrete droplets called GEMs (“Gel Beads in Emulsion”). Once generated, the GEMs are collected, and a heat incubation step is carried out. The heating step facilitates release of the cell contents and RNA, capture of RNA by barcode oligonucleotides, and the reverse-transcription (RT) reaction that results in cDNA synthesis incorporating the barcodes in the 3′ synthons. cDNA electropherogram analysis was performed using Agilent 2100 Bioanalyzer 5067-4626 to assess DNA size and yield from each sample.

### Results

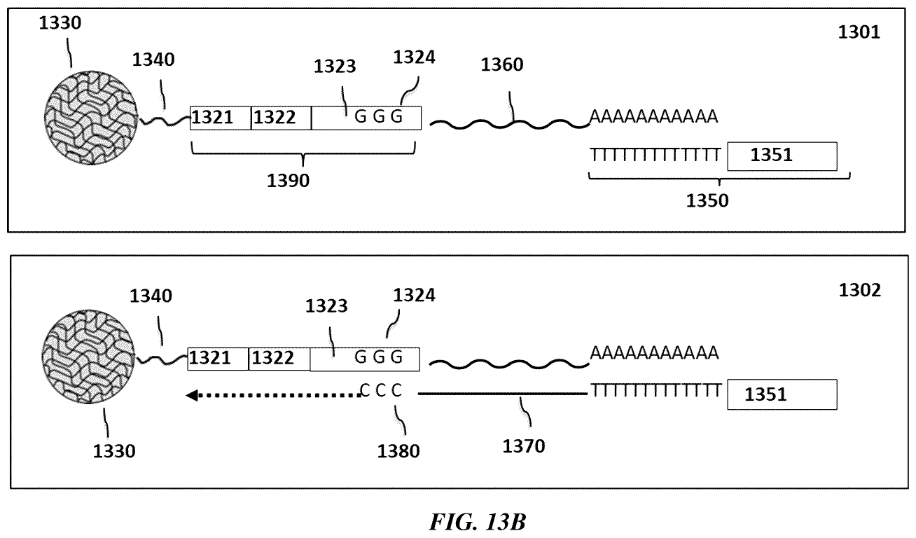

As shown by the results depicted in the comparative plots of FIGS. 14A and 14B, the chop-fixed kidney tissue sample preparation relative to the “dissociated-then-fixed” kidney tissue sample exhibited a higher percentage genes detected less deterioration at day 1 relative day 6. Furthermore, as shown by the results depicted in the plots of FIG. 14C, the chop-fixed brain tissue showed a significant increase in both genes and UMIs detected relative to a fresh brain tissue sample.

### Example 3: ATAC-Seq and Gene Expression Analysis of Chop-Fixed Mouse Brain Tissue

This example illustrates a study of the use of the chop-fixed method to prepare mouse brain tissue samples for analysis with a range of single-cell/single nucleus analysis methods, including ATAC analysis.

### Materials and Methods

### A. Sample Preparation:

Chop-fixed cell samples were prepared from mouse brain tissue as described in Example 1. The “Fresh control” brain tissue sample was prepared as described in Example 2. “Dissociated-then-fixed” brain tissue samples were prepared without chopping but simply by treatment of the tissue with dissociating reagent followed by fixing in 4% PFA.

### B. Multiome Analysis:

Brain tissue nuclei were prepared in three distinct ways: 1) Fresh nuclei were isolated directly from E18 brain tissue; 2) Fresh nuclei from E18 brain tissue were fixed then treated with the un-fixing agent of compound (8) as described above for a fixed cell sample preparation; or 3) Chop-fixed E18 brain tissue was extracted for fixed nuclei using the same method as fresh nuclei samples then treated with the un-fixing agent of compound (8). The three brain tissue preparations were loaded into the Chromium instrument (10× Genomics), processed using the Single Cell Multiome ATAC+Gene Expression solution (10× Genomics) and analyzed using Cell Ranger ARC pipeline (10× Genomics), which includes single cell/single nucleus 3′ gene expression paired with ATAC sequencing.

### Results

As shown by the results summarized in Table 2 (below), the “chop-fixed then isolated” E18 brain tissue sample preparation relative to the “dissociated-then-fixed” brain tissue sample exhibited a substantially higher percentage of genes and UMIs detected (relative to fresh control samples). Moreover, the chop-fixed brain tissue preparation exhibited a substantially higher percentage of unique ATAC fragments than the “dissociated-then-fixed” or even the fresh control sample.

TABLE 2

| | | Dissociated | Chop fixed then |
|---|---|---|---|
| | Fresh E18 | then fixed E18 | isolated E18 |
| | brain nuclei | brain nuclei | brain nuclei |
| Median genes per cell | 1880 | 1110 | 1376 |
| (20krrpc) | | | |
| Median UMIs per cell | 3619 | 1964 | 2428 |
| (20krrpc) | | | |
| Unique ATAC | 8382 | 2786 | 10784 |
| fragments per cell | | | |

### Example 4: Multiome Analysis of Chop-Fixed then Un-Fixed Mouse Brain Tissue

This example illustrates a study of the use of the chop-fixed method to prepare mouse brain tissue samples, which are then un-fixed using an un-fixing agent of compound (8), followed by analysis of the un-fixed samples with a range of single-cell analysis methods, including ATAC analysis.

### Materials and Methods

### A. Fixed Cell Sample Preparation:

Chop-fixed cell samples were prepared from mouse brain tissue as described in Example 1. “Fresh nuclei” brain tissue samples were prepared in same manner as “Fresh control” samples described in Example 2.

### B. Preparation of Un-Fixing Agent Compound (8)

The un-fixing agent of compound (8) was prepared using the following 2-step synthesis procedure.

Step 1: Diethyl (4-aminopyridin-3-yl)phosphonate. In step 1 the compound, diethyl (4-aminopyridin-3-yl)phosphonate was prepared according to the procedure described in Guilard, R. et al. Synthesis, 2008, 10, 1575-1579. Briefly, to a solution of 3-bromopyridine-4-amine (2.5 g, 14.5 mmol, 1 equiv) (CAS: 13534-98-0, Sigma Aldrich) in ethanol (58 mL) was added diethyl phosphite (2.2 mL, 17.3 mmol, 1.2 equiv.) triethylamine (3 mL, 1.5 equiv), PPh3 (1.1 g, 4.3 mmol, 30 mol %) and Pd(OAc)2 (0.39 g, 1.73 mmol, 12 mol %). The reaction mixture was purged with Argon for 5 min. After heating to reflux for 24 h, the reaction mixture was cooled to room T and conc. in vacuo. The residue was purified by silica gel chromatography (MeOH/DCM) to give the title compound (0.35 g, 11% yield). 1H NMR (80 MHz, CDCl3): δ=1.15 (t, 6H, CH3), 4.18-3.69 (m, 4H, CH2), 5.99 (br-s, 2H, NH2), 6.49 (d, 1H), 8.03-7.93 (m, 1H), 8.22 (d, 1H).

Step 2: (4-Aminopyridin-3-yl)phosphonic acid (compound (8). In step 2, the target compound, (4-Aminopyridin-3-yl)phosphonic acid (compound (8)) was prepared by acid hydrolysis of the precursor compound of step 1. Diethyl (4-aminopyridin-3-yl)phosphonate (0.35 g, 1.52 mmol, 1 equiv) was suspended in 6 N HCl (aq.) (8 mL). After refluxing for 12 h, the reaction mixture was conc. in vacuo. The residue was washed with DCM, ether and conc in vacuo to afford the target compound (8) (247 mg, 93% yield). 1H NMR (80 MHz, D2O): δ=6.85-6.55 (m, 1H), 8.05-7.94 (m, 1H), 8.40-8.26 (m, 1H).

A stock solution of the un-fixing agent of 300 mM compound (8) in 50 mM Tris-HCl, 1 mM EDTA, pH 8.3, was prepared, filtered using a 5 μm syringe filter, and stored at room temperature.

### C. Sample Un-Fixing Treatment

RNAse inhibitor was added to the chop-fixed cell solution of step A, together with 100 mM of the un-fixing agent, compound (8), and 10 U of the cold-active protease, ArcticZymes Proteinase. The un-fixing mixture was allowed to incubate at 8° C. for 2 h, followed by a higher temperature incubation at 70° C. for 15 min. The resulting un-fixed cell solution was spun down for 5 minutes at 500 g, 4° C., and the supernatant and pellet fractions were collected separately. The “Fresh nuclei” samples were not treated with any un-fixing agent or protease.

### D. Multiome Analysis:

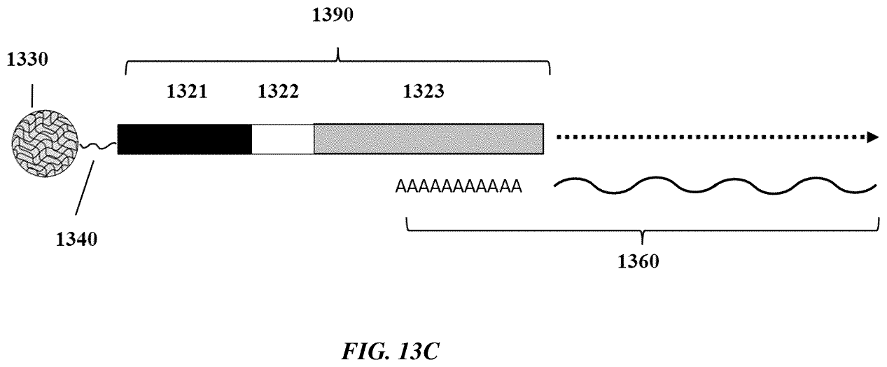

Brain tissue nuclei were prepared in three distinct ways: 1) Fresh nuclei were isolated directly from E18 brain tissue; 2) Fresh nuclei from E18 brain tissue were fixed then treated with the un-fixing agent of compound (8) as described above for a fixed cell sample preparation; or 3) Chop-fixed E18 brain tissue was extracted for fixed nuclei using the same method as fresh nuclei samples then treated with the un-fixing agent of compound (8). The three brain tissue preparations were loaded into the Chromium instrument (10× Genomics), processed using the Single Cell Multiome ATAC+Gene Expression solution (10× Genomics), and analyzed using Cell Ranger ARC pipeline (10× Genomics), which includes single cell/single nucleus 3′ gene expression paired with ATAC sequencing. The individual gene expression and ATAC metrics were calculated as well as combined RNA/ATAC sequencing metrics as shown in FIGS. 15A and 15B.

### Results

As shown by the results depicted in the comparative plots of FIGS. 15A and 15B, ATAC transposition events from the chop-fixed brain tissue nuclei sample preparation that was un-fixed using the un-fixing agent of compound (8) exhibited much cleaner separation relative to the plot of transposition events from the “Fresh nuclei” samples. This separation observed for the chop-fixed brain tissue that was un-fixed corresponds to only 31% of ATAC fragments overlapping with called peaks, whereas 55% of “fresh nuclei” fragments overlapped with other called peaks. These results suggest that chop-fixed samples that are un-fixed using an un-fixing agent can provide higher quality ATAC data from their nuclei than can be obtained from fresh cells.

### Example 5: Single-Cell 3′ Sequence of Analysis of Chop-Fixed then Un-Fixed Mouse Kidney Tissue

This example illustrates a study of the use of the chop-fixed method to prepare mouse kidney tissue samples, which are then un-fixed using an un-fixing agent of compound (8), followed by single-cell 3′ sequence analysis of the un-fixed samples.

### Materials and Methods

### A. Fixed Cell Sample Preparation:

Chop-fixed cell samples were prepared from mouse kidney tissue as described in Example 1. “Fresh control” kidney tissue samples were prepared as described in Example 2.

### B. Sample Un-Fixing Treatment

Chop-fixed kidney tissue were stored for up to 6 days in PBS+RNase inhibitor and 0.04% BSA. On the day of analysis fixed kidney tissue samples were dissociated then un-fixing treatment of the samples of step A were carried using the un-fixing treatment described in Example 4, then analyzed by single cell 3′ gene expression.

### C. Single-Cell 3′ Sequence Analysis

The single-cell 3′ sequence analysis of the un-fixed samples was carried out as described in Example 2.

### D. Cell Counting

Determination and mapping of cell types present in the chop-fixed kidney tissue samples that were un-fixed with compound (8) was carried out by automated meta-analysis of cell clusters identified using differentially expressed marker gene expression. Tissue cell type composition was identified by an automated script that quantifies the number and fraction of cell types known to be detected in kidney tissue samples by categorizing cells based on a combination of differentially expressed known marker genes for each cell type, with unclassified cells going to the undetermined category.

### Results

As shown by the results summarized in Table 3 (below), kidney tissue that was chop-fixed, dissociated, un-fixed, and stored for up to 6 days, was found to show reasonable sensitivity in various single cell 3′ sequence analysis metrics relative to cells from freshly dissociated kidney tissue. The single cell sequencing data was down-sampled to normalize for sequencing depth differences at a depth of 30,000 raw reads per cell (rrpc).

TABLE 3

| | Day 1 30k rrpc | Day 6 30k rrpc |
|---|---|---|
| Metrics | (vs Fresh) | (vs Fresh) |
| Median UMIs per cell | 24% | 18% |
| Median genes per cell | 76% | 61% |
| Fraction of reads in cells | 76% (vs. 47% in D 0 fresh) | 66% |

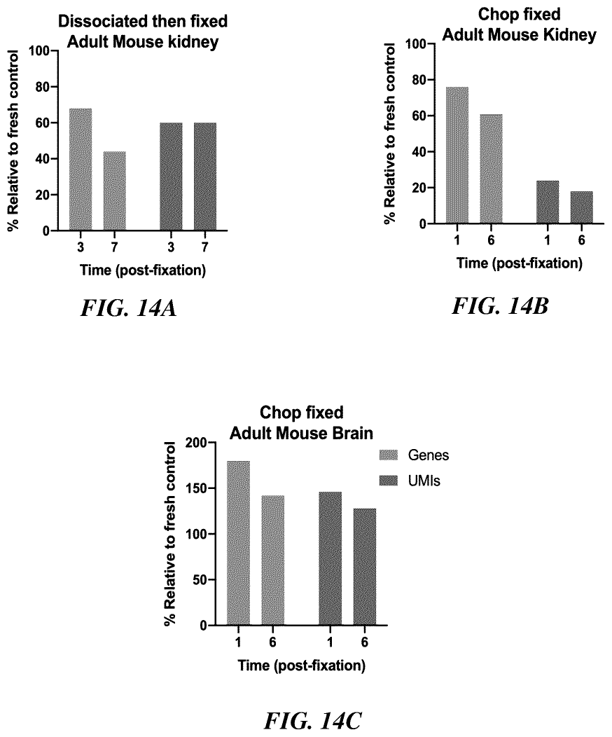

As shown by the results depicted in the plots of FIG. 16, the kidney tissue that was chop-fixed, dissociated, un-fixed, and stored for up to 6 days, was found to exhibit more robust cell type clusters compared to fresh tissue samples, including greater presence of 15 out of 24 clusters, including more of the following cell types: distal tubules, collecting duct, and interstitium. Additionally, the chop-fixed then un-fixed kidney tissue samples exhibited minimal heat shock and cellular stress gene signatures (Jun, Hspa4, Gadd45b, and Nr4a1) compared to fresh cells which were treated with collagenase digestion at a 37 C.

### Example 6: Single-Cell 3′ Sequence of Analysis of Chop-Fixed then Un-Fixed Mouse Brain Tissue

This example illustrates a study of the use of the chop-fixed method to prepare mouse brain tissue samples, which are then un-fixed using an un-fixing agent of compound (8), followed by single-cell 3′ sequence analysis of the un-fixed samples.

### Materials and Methods

### A. Fixed Cell Sample Preparation:

Chop-fixed cell samples were prepared from mouse brain tissue as described in Example 1.

### B. Sample Un-Fixing Treatment

Un-fixing treatment of the fixed brain tissue samples of step A were carried using the un-fixing treatment described in Example 4.

### C. Single-Cell 3′ Sequence Analysis

The single-cell 3′ sequence analysis of the un-fixed samples was carried out as described in Example 2.

### D. Cell Counting

Determination and mapping of cell types present in the chop-fixed brain tissue samples that were un-fixed with compound (8) was carried out in the same manner as described for kidney in Example 5.

### Results

As shown by the results summarized in Table 4 (below), brain tissue that was chop-fixed, dissociated, un-fixed, and stored for up to 6 days, was found to show greatly improved sensitivity in various single cell 3′ sequence analysis metrics relative to cells from freshly dissociated brain tissue.

TABLE 4

| | Day 1 30k rrpc | Day 6 30k rrpc |
|---|---|---|
| Metrics | (vs Fresh) | (vs Fresh) |
| Median UMIs per cell | 146% | 128% |
| Median genes per cell | 180% | 142% |
| Fraction of reads in cells | 73% (vs. 51% in D 0 fresh) | 64% |

Results of further cell type counting and mapping analysis of brain cell types present in the samples are summarized in Table 5 (below).

TABLE 5

| | Median genes per cell | Median UMIs per cell |
|---|---|---|
| | (30k rrpc) | (30k rrpc) |
| Endo (fresh) | 800 | 1800 |
| Endo (fixed) | 500 | 800 |
| Glial (fresh) | 1250 | 2900 |
| Glial (fixed) | 800 | 1400 |
| Neurons (fresh) | 500 | 900 |
| Neurons (fixed) | 1600 | 2900 |

The results summarized in Table 5 show that genes and UMIs from neurons are much more prevalent in the chop-fixed then un-fixed samples than fresh, whereas representation of endo and glial cells from the brain tissue are more prevalent in the fresh samples. Not only are the neurons more abundant in the chop-fixed brain tissue preparation but they exhibit up to 3× more complexity. Additionally, like the samples from kidney tissue, samples of cells prepared from chop fixed brain tissue show far less heat-induced stress response genes (Jun, Fox, Dusp1, and Nr4a1) compared to freshly dissociated cells.

### Example 7: Chop-Fix Methods Used to Prepare Human Uterine Tissue for Single-Cell Sequence Analysis

This example illustrates a study of the use of a chop-fixed method to prepare human uterine tissue samples for further analysis using a single-cell RNA-based sequencing method.

### Materials and Methods

Fresh tissue was collected from normal uterine tissue adjacent to tissue exhibiting endometrial cancer. The tissue was surgically removed and packed in ice for laboratory for analysis within 16-24 hours post-surgery. A shipping delay, however, resulted in the tissue being received in the laboratory about 48 hours post-surgery, with the tissue at room temperature.

The tissue was sectioned into smaller pieces before subjecting to the seven different chop-fix and/or other preparation methods for downstream single cell assays as described below.

Chop-fixing of the tissue sections was carried out as described above in Examples 1-6. After chopping, the tissue was fixed for 16-24 hours in a 4% paraformaldehyde solution at 4° C. After fixation, the chop-fixed tissue was centrifuged at 850 rcf for 5 min at room temperature, and supernatant was removed without disturbing the fixed tissue pellet. The fixed tissue pellet was dissociated as described below.

A Dissociation Solution of RPMI+0.2 mg/mL Liberase was prepared by adding 80 pL of Liberase stock solution into 1,920 μL to RPMI and mixing. This Dissociation Solution was stored at 4° C., and then pre-warmed for 10 min at 37° C. before use.

2 mL of the pre-warmed Dissociation Solution was added to the fixed tissue sample. The sample was dissociated using an Octo Dissociator or Manual dissociation as described below.

Octo Dissociator: Transfer sample to Miltenyi C tubes. The minimum Dissociation Solution required for using the C tubes is 2 mL. Place the C tube in the Octo Dissociator and run following program: Incubate for 20 min at 37° C., 50 rpm; Spin for 30 sec at 37° C., 2,000 rpm—clockwise; Spin for 30 sec at 37° C., 2,000 rpm—anticlockwise. The C tubes are detached and the dissociated tissue through a 70 μm filter to remove debris and undissociated tissue pieces.

Manual Dissociation: Incubate the sample for 20 min at 37° C. shaking the tube intermittently. Using a silanized glass pipette, triturate the tissue pieces 15-20× (until solution begins to turn cloudy) to obtain a single cell suspension. Pass the dissociated tissue through a 70 μm filter to remove debris and undissociated tissue pieces.

Following either dissociation protocol, the sample is centrifuged at 850 rcf for 5 min, and the supernatant is removed without disturbing the pellet. The pellet is resuspended in 1 mL or chilled buffer.

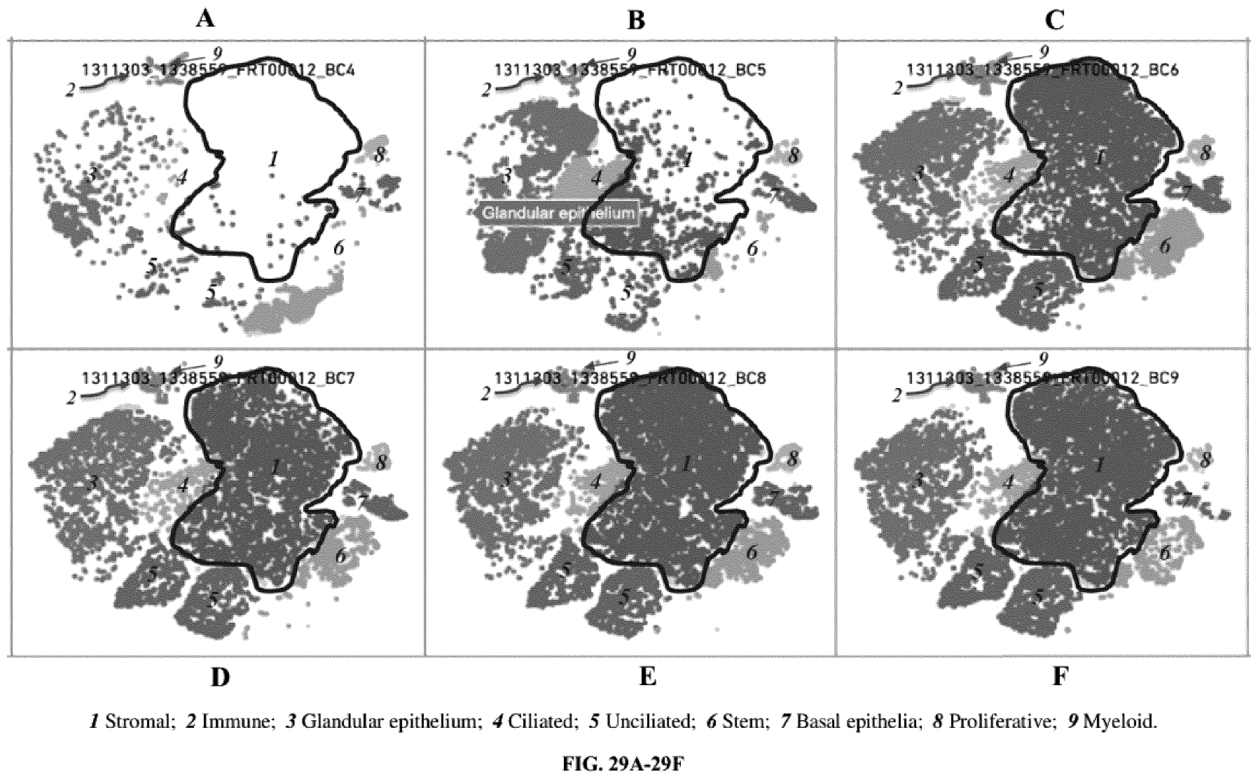

For the present comparative study, a total of seven different uterine tissue samples were prepared and analyzed: (1) tissue dissociated to single cells without any fixation and then subjected to the 10× Genomics single cell 3′ v3 assay (data not shown); (2) tissue dissociated to single cells then subjected to the 10× Genomics fixed RNA profiling assay (results shown in FIG. 29A); (3) flash freezing of fresh tissue followed by processing of the flash frozen tissue with the 10× Genomics nuclei isolation kit, followed by subjecting the isolated nuclei to the 10× Genomics fixed RNA profiling assay (results shown in FIG. 29B); (4) fresh tissue chop-fixed and then dissociated to single cells (results shown in FIG. 29C); (5) fresh tissue flash-frozen, then chop-fixed, then dissociated to single cells (results shown in FIG. 29D); (6) fresh tissue chop-fixed, then stored at 4° C. for 5 days, then dissociated to single cells and subjected to 10× Genomics fixed RNA profiling assay (results shown in FIG. 29E); and (7) fresh tissue flash frozen, then chop-fixed, then stored at 4° C. for 5 days, then dissociated to single cells and subjected to the 10× Genomics fixed RNA profiling assay (results shown in FIG. 29F).

### Results

FIG. 29A-29F depicts plots of the cell-type clusters detected in the seven preparations of human uterine tissue samples. It should be noted that cells prepared without any fixation and assayed using the standard 10× Genomics single cell 3′ v3 assay did not yield any usable data for preparing such cell cluster plots. Comparison of the plots shows that at least nine different cell types can be identified using the RNA profiling assays used in the study, however, the detection of certain cell type clusters differs greatly. It is abundantly clear from a comparison of plots of FIGS. 29A-29F that many of the cell types are underrepresented or absent of the standard freshly dissociated sample that was subjected to the 10× Genomics RNA profiling assay (FIG. 29A) when compared to the samples that are chop-fixed then dissociated and subjected to the whole-cell 10× Genomics fixed RNA profiling assay (FIG. 29C-29F). The most apparent absence is among the stromal cells, which fall within the outlined section “1” in each of the panels of FIGS. 29A-29F. Further, as shown by the results depicted in FIG. 29E and FIG. 29F, human uterine tissue can be chop-fixed and stored at 4° C. for 5 days, before enzymatically dissociating the chop-fixed tissue and subjecting it to single-cell RTL analysis. The data resulting from the cells that were stored for 5 days showed no loss of sample complexity as compared with samples that were on the day of immediate processing.

In summary, the chop-fix method provided single-cell samples that were capable of returning sufficient data for calling/identifying numerous cell types in the sample, whereas single-cell samples prepared using no fixation resulted in a complete inability to generate RNA data, and single-cell samples prepared utilizing just a dissociation of the tissue (common in tissue processing labs) prior to subjecting to the 10× Genomics fixed RNA profiling assay revealed a very poor outcome regarding the number of RNA transcripts and the ability to discern the different types of cells from one another (see FIG. 29A vs. FIGS. 29C-29F). These results demonstrate that the chop-fix methods described herein can be used to prepare human tissue samples suitable for single-cell RNA profiling assays, even from a tissue sample that has suffered under less than ideal conditions due a shipping delay. The chop-fix methods for tissue sample preparation thus allow for the ability to prepare samples that can more easily withstand normally unfavorable shipping (or storage) times and conditions and thereby provide favorable single-cell analysis of a shipped and/or stored sample at a distant site.

While the foregoing disclosure has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the disclosure are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

1. A method for analysis of a biological tissue comprising:
(a) chopping the biological tissue to provide a composition of tissue fragments;
(b) treating the composition of tissue fragments with a solution comprising a fixation agent to provide a composition of fixed tissue fragments;
(c) treating the composition of fixed tissue fragments with a cell dissociation reagent to provide a composition of fixed cells, each fixed cell comprising a plurality of crosslinked nucleic acid molecules; and
(d) generating a plurality of barcoded nucleic acid molecules from the plurality of crosslinked nucleic acid molecules and a plurality of nucleic acid barcode molecules, wherein a barcoded nucleic acid molecule of the plurality of barcoded nucleic acid molecules comprises: i) a sequence corresponding to a crosslinked nucleic acid molecule of the plurality of crosslinked nucleic acid molecules or a complement thereof, and ii) a barcode sequence or a complement thereof.

2. The method of claim 1, wherein the generating the plurality of barcoded nucleic acid molecules is performed in a plurality of partitions.

3. The method of claim 2, wherein a partition of the plurality of partitions comprises a fixed cell and a support comprising the plurality of nucleic acid barcode molecules.

4. The method of claim 1, wherein the barcode sequence is a partition-specific barcode sequence.

5. The method of claim 1, wherein the composition of tissue fragments comprises particles of an average size on a side of about 500 μm or less.

6. The method of claim 1, wherein the fixing agent is paraformaldehyde (“PFA”).

7. The method of claim 1, wherein the cell dissociation reagent comprises collagenase.

8. The method of claim 1, wherein an amount of time prior to treating the fixed tissue fragments with the cell dissociation reagent is at least 1 hour.

9. The method of claim 1, further comprising filtering or sieving the composition of fixed cells.

10. A method for analysis of a biological tissue comprising:
(a) chopping the biological tissue to provide a composition of tissue fragments;
(b) treating the composition of tissue fragments with a solution comprising a fixation agent to provide a composition of fixed tissue fragments;
(c) treating the composition of fixed tissue fragments with a cell dissociation reagent to provide a composition of fixed cells, each fixed cell comprising a plurality of crosslinked nucleic acid molecules;
(d) treating the composition of fixed cells with an un-fixing agent to provide a composition of un-fixed cells, each un-fixed cell comprising a plurality of un-crosslinked nucleic acid molecules; and
(e) generating a plurality of barcoded nucleic acid molecules from the plurality of un-crosslinked nucleic acid molecules and a plurality of nucleic acid barcode molecules, wherein a barcoded nucleic acid molecule of the plurality of barcoded nucleic acid molecules comprises i) a sequence corresponding to an un-crosslinked nucleic acid molecule of the plurality of un-crosslinked nucleic acid molecules or a complement thereof, and ii) a barcode sequence or a complement thereof.

11. The method of claim 10, wherein the generating the plurality of barcoded nucleic acid molecules is performed in a plurality of partitions.

12. The method of claim 11, wherein a partition of the plurality of partitions comprises an un-fixed cell and a support comprising the plurality of nucleic acid barcode molecules.

13. The method of claim 10, wherein the treating with the un-fixing agent is performed in a plurality of partitions.

14. The method of claim 13, wherein a partition of the plurality of partitions comprises an un-fixed cell and a support comprising the plurality of nucleic acid barcode molecules.

15. The method of claim 10, wherein the barcode sequence is a partition-specific barcode sequence.

16. The method of claim 10, wherein the composition of tissue fragments comprises particles of an average size on a side of about 500 μm or less.

17. The method of claim 10, wherein the fixing agent is paraformaldehyde (“PFA”).

18. The method of claim 10, wherein the cell dissociation reagent comprises collagenase.

19. The method of claim 10, wherein an amount of time prior to treating the fixed tissue fragments with the cell dissociation reagent is at least 1 hour.

20. The method of claim 10, further comprising filtering or sieving the composition of fixed cells or the composition of un-fixed cells.
